SEQUENCE LISTING (27 sequences)

SEQ ID NO: 1 - protein, 30 aa, synthetic construct
  source 1..30: note = 2G10-5_2G10-5-CaaX_2G10-6_HFR1
QVQLVQSGAEVKKPGASVKVSCKASGFTFS

SEQ ID NO: 2 - protein, 5 aa, synthetic construct
  source 1..5: note = 2G10-5_2G10-5-CaaX_2G10-6_HCDR1
SSYIS

SEQ ID NO: 3 - protein, 14 aa, synthetic construct
  source 1..14: note = 2G10-5_2G10-5-CaaX_2G10-6_HFR2
WLRQAPGQRLEWIA

SEQ ID NO: 4 - protein, 17 aa, synthetic construct
  source 1..17: note = 2G10-5_2G10-5-CaaX_2G10-6_HCDR2
WIYAGTGGTSYNQKFTG

SEQ ID NO: 5 - protein, 32 aa, synthetic construct
  source 1..32: note = 2G10-5_2G10-5-CaaX_HFR3
KATLTVDTSASTAYMELSSLRSEDTAVYYCAR

SEQ ID NO: 6 - protein, 10 aa, synthetic construct
  source 1..10: note = 2G10-5_2G10-5-CaaX_2G10-6_HCDR3
HNPRYYAMDY

SEQ ID NO: 7 - protein, 11 aa, synthetic construct
  source 1..11: note = 2G10-5_2G10-5-CaaX_2G10-6_HFR4
WGQGTTVTVSS

SEQ ID NO: 8 - protein, 23 aa, synthetic construct
  source 1..23: note = 2G10-5_2G10-5-CaaX_2G10-6_LFR1
DTQMTQSPSSLSASVGDRVTITC

SEQ ID NO: 9 - protein, 11 aa, synthetic construct
  source 1..11: note = 2G10-5_2G10-5-CaaX_2G10-6_LCDR1
ITSTDIDDDMN

SEQ ID NO: 10 - protein, 15 aa, synthetic construct
  source 1..15: note = 2G10-5_2G10-5-CaaX_2G10-6_LFR2
WYQQKPGKAPKLLIS

SEQ ID NO: 11 - protein, 7 aa, synthetic construct
  source 1..7: note = 2G10-5_2G10-5-CaaX_2G10-6_LCDR2
EGNTLRP

SEQ ID NO: 12 - protein, 32 aa, synthetic construct
  source 1..32: note = 2G10-5_2G10-5-CaaX_2G10-6_LFR3
GVPSRFSGSGYGTDFTFTISSLQPEDIATYYC

SEQ ID NO: 13 - protein, 9 aa, synthetic construct
  source 1..9: note = 2G10-5_2G10-5-CaaX_2G10-6_LCDR3
LQSDNLPYT

SEQ ID NO: 14 - protein, 10 aa, synthetic construct
  source 1..10: note = 2G10-5_2G10-5-CaaX_2G10-6_LFR4
FGGGTKVEIK

SEQ ID NO: 15 - protein, 119 aa, synthetic construct
  source 1..119: note = 2G10-5_2G10-5-CaaX_VH
QVQLVQSGAEVKKPGASVKVSCKASGFTFSSSYISWLRQAPGQRLEWIAWIYAGTGGTSYNQKFTGKATLTVDTSASTAYMELSSLRSEDTAVYYCARHNPRYYAMDYWGQGTTVTVSS

SEQ ID NO: 16 - protein, 107 aa, synthetic construct
  source 1..107: note = 2G10-5_2G10-5-CaaX_2G10-6_VL
DTQMTQSPSSLSASVGDRVTITCITSTDIDDDMNWYQQKPGKAPKLLISEGNTLRPGVPSRFSGSGYGTDFTFTISSLQPEDIATYYCLQSDNLPYTFGGGTKVEIK

SEQ ID NO: 17 - protein, 449 aa, synthetic construct
  source 1..449: note = 2G10-5_2G10-5-CaaX_HC
QVQLVQSGAEVKKPGASVKVSCKASGFTFSSSYISWLRQAPGQRLEWIAWIYAGTGGTSYNQKFTGKATLTVDTSASTAYMELSSLRSEDTAVYYCARHNPRYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, synthetic construct
  source 1..214: note = 2G10-5_2G10-6_LC
DTQMTQSPSSLSASVGDRVTITCITSTDIDDDMNWYQQKPGKAPKLLISEGNTLRPGVPSRFSGSGYGTDFTFTISSLQPEDIATYYCLQSDNLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 32 aa, synthetic construct
  source 1..32: note = 2G10-6_HFR3
RVTLTVDTSASTAYMELSSLRSEDTAVYYCAR

SEQ ID NO: 20 - protein, 119 aa, synthetic construct
  source 1..119: note = 2G10-6_VH
QVQLVQSGAEVKKPGASVKVSCKASGFTFSSSYISWLRQAPGQRLEWIAWIYAGTGGTSYNQKFTGRVTLTVDTSASTAYMELSSLRSEDTAVYYCARHNPRYYAMDYWGQGTTVTVSS

SEQ ID NO: 21 - protein, 449 aa, synthetic construct
  source 1..449: note = 2G10-6_HC
QVQLVQSGAEVKKPGASVKVSCKASGFTFSSSYISWLRQAPGQRLEWIAWIYAGTGGTSYNQKFTGRVTLTVDTSASTAYMELSSLRSEDTAVYYCARHNPRYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 11 aa, synthetic construct
  source 1..11: note = CaaX peptide moiety
GGGGGGGCVIM

SEQ ID NO: 23 - protein, 225 aa, synthetic construct
  source 1..225: note = 2G10-5-CaaX_LC
DTQMTQSPSSLSASVGDRVTITCITSTDIDDDMNWYQQKPGKAPKLLISEGNTLRPGVPSRFSGSGYGTDFTFTISSLQPEDIATYYCLQSDNLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGGGCVIM

SEQ ID NO: 24 - protein, 4 aa, synthetic construct
CVIM

SEQ ID NO: 25 - protein, 4 aa, synthetic construct
CVLL

SEQ ID NO: 26 - protein, 8 aa, synthetic construct
  CROSSLNK 1..5: note = Tryptathionine linkage between 6-hydroxy-tryptophan and S-oxy cysteine
  MOD_RES 1: note = 6-hydroxy-tryptophan
  MOD_RES 5: note = S-oxy cysteine
  MOD_RES 7: note = 4Hyp
  MOD_RES 8: note = 4,5-dihydroxyisoleucine
WGIGCNPI

SEQ ID NO: 27 - protein, 8 aa, synthetic construct
  CROSSLNK 4..8: note = Tryptathionine linkage between 6-hydroxy-tryptophan and S-oxy cysteine
  MOD_RES 1: note = 4,5-dihydroxyisoleucine
  MOD_RES 2: note = 4Hyp
  MOD_RES 4: note = S-oxy cysteine
  MOD_RES 8: note = 6-hydroxy-tryptophan
IPNCGIGW

(19) United States
(12) Patent Application Publication
(10) Pub. No.: US 2025/0242043 A1
(43) Pub. Date: Jul. 31, 2025

(54) ANTIBODY-DRUG CONJUGATE COMPRISING ANTIBODY AGAINST HUMAN TROP2 AND USE THEREOF

(71) Applicant: LigaChem Biosciences Inc., DAEJEON (KR)

(72) Inventors: Chang Sik Park, Daejeon (KR); Ho Young Song, Daejeon (KR); Hye Jung Kim, Yuseong-gu (KR); Chul-Woong Chung, Daejeon (KR); Saverio Alberti, Lanciano (Chieti) (IT)

(21) Appl. No.: 18/854,516

(22) PCT Filed: Apr. 5, 2023
(86) PCT No.: PCT/IB23/00193

§ 371 Date: Oct. 4, 2024

(30) Foreign Application Priority Data
Apr. 6, 2022 (KR) 10-2022-0043049

(51) Int. Cl. A61K 47/68; A61K 9/00; A61K 45/06; A61P 35/00; C07K 16/30

(52) U.S. Cl. CPC A61K 47/6851; A61K 9/0019; A61K 45/06; A61K 47/68033; A61K 47/68035; A61K 47/6889; A61P 35/00; C07K 16/30

(57) ABSTRACT

The present disclosure relates to an antibody-drug conjugate (ADC) targeting human TROP2 (tumor-associated calcium signal transducer 2, TACSTD2)) and use thereof, and more particularly, to: an ADC including an antibody or antigen-binding fragment thereof that binds to human TROP2; and use of ADCs for producing a drug for the treatment and/or treatment of diseases, more particularly, hyperproliferative and/or angiogenic diseases, such as cancer diseases.

### RELATED APPLICATIONS

This application is a 371 U.S. National Stage Application of PCT/IB23/00193, filed on Apr. 4, 2023, which claims the benefit of priority to Korean Patent Application 10-2022-0043049, filed on Apr. 6, 2022; the entire contents of each of said applications are incorporated herein in their entirety by reference.

### BACKGROUND

Cancer refers to a disease caused by abnormally grown lumps due to autonomous overgrowth of body tissues, and is the result of uncontrolled cell growth in various tissues. Tumors in early stage can be removed by surgical and radio-therapeutic measures, and metastasized tumors are generally treated using chemotherapy.

Most chemotherapeutic agents administered parenterally may induce unwanted side effects and even serious toxicity, as a result of systemic administration. Accordingly, the focus of development has been on developing treatments to achieve increased efficacy and/or reduced minimal toxicity/side effects, for example through the selective application of these chemotherapeutic agents in tumor cells or immediately adjacent tissues.

### SUMMARY OF THE DISCLOSURE

In certain aspects, the present disclosure provides antibody-drug conjugates comprising an anti-TROP2 antibody that binds to TROP2. In certain embodiments, the antibody disclosed herein binds to TROP expressed in a tumor and may be used to deliver a drug to the tumor. In certain embodiments, the antibody drug conjugates disclosed herein have improved stability as compared to antibody drug conjugates known in the art.

In certain aspects, the present disclosure provides conjugates having a structure represented by General Formula I or a pharmaceutically acceptable salt thereof:

Ab-[L-(B)₁]ₘ [General Formula I]
wherein,
Ab is an anti-TROP2 (tumor-associated calcium signal transducer 2 aka TACSTD2) antibody or antigen-binding fragment thereof comprising a heavy chain variable region and a light chain variable region wherein:
   the heavy chain variable region comprises a heavy chain CDR1 comprising the amino acid sequence of SEQ ID NO: 2, a heavy chain CDR2 comprising the amino acid sequence of SEQ ID NO: 4, a and heavy chain CDR3 comprising the amino acid sequence of SEQ ID NO: 6; and
   the light chain variable region comprises a light chain CDR1 comprising the amino acid sequence of SEQ ID NO: 9, a light chain CDR2 comprising the amino acid sequence of SEQ ID NO: 11, and a light chain CDR3 comprising the amino acid sequence of SEQ ID NO: 13;
L is a linker;
B is an active agent moiety, and
1 and m are each independently an integer selected from 1 to 20.

### BRIEF DESCRIPTION OF DRAWINGS

FIG. 1 is a schematic view illustrating a method of preparing an antibody-drug conjugate ADC1 according to an embodiment of the present disclosure.

FIG. 2 is a schematic view illustrating a method of preparing an antibody-drug conjugate ADC2 according to an embodiment of the present disclosure.

FIG. 3 is a schematic view illustrating a method of preparing an antibody-drug conjugate ADC3 according to an embodiment of the present disclosure.

FIG. 4 is a schematic view illustrating a method of preparing an antibody-drug conjugate ADC4 according to an embodiment of the present disclosure.

FIGS. 5A & 5B illustrate the results of confirming the tumor growth inhibitory efficacy of an antibody-drug conjugate according to an embodiment of the present disclosure in a mouse model transplanted with pancreatic cancer cell line BxPC-3 (FIG. 5A) and a mouse model transplanted with breast cancer cell line MDA-MB-468 (FIG. 5B) through a first in-vivo experiment.

FIGS. 6A-6D illustrate the results of confirming the tumor growth inhibitory efficacy of an antibody-drug conjugate according to an embodiment of the present disclosure in a mouse model transplanted with breast cancer cell line MDA-MB-468 (FIG. 6A), a mouse model transplanted with pancreatic cancer cell line BxPC-3 (FIG. 6B), a mouse model transplanted with gastric cancer cell line NCI-N87 (FIG. 6C), and a mouse model transplanted with non-small cell lung cancer cell line HCC827 (FIG. 6D) through a second in-vivo experiment.

FIGS. 7A-7E illustrate the results of confirming the tumor growth inhibitory efficacy of an antibody-drug conjugate according to an embodiment of the present disclosure in a mouse model transplanted with pancreatic cancer cell line BxPC-3 (FIG. 7A), a mouse model transplanted with non-small cell lung cancer cell line HCC827 (FIG. 7B), a mouse model transplanted with pancreatic cancer cell line Capan-1 (FIG. 7C), a mouse model transplanted with non-small cell lung cancer cell line NCI-H2170 (FIG. 7D), and a mouse model transplanted with colon cancer cell line HT29 (FIG. 7E).

### DETAILED DESCRIPTION OF DISCLOSURE

An antibody-drug conjugate (ADC) is a targeted technology for conjugating a toxin or a drug to an antibody that binds to an antigen, by which the toxin is released in a cell to cause death of cancer cells and the like. The ADC enables a drug to be accurately delivered to target cancer cells while minimally affecting healthy cells, and to be released only under specific conditions, and thus has excellent efficacy compared to antibody therapeutic agents themselves and can remarkably reduce the risk of side effects compared to existing anticancer agents.

The basic structure of these antibody-drug conjugates is “antibody-linker-small molecule drug (toxin)”. In this structure, the linker play a functional role in linking the antibody and the drug, but in some cases also ensures that the drug is released from the antibody at the appropriate time, for example after reaching target cells. That is, the stability of the linker can play a very important role in the efficacy and safety such as systemic toxicity of an antibody-drug conjugate (Discovery Medicine 2010, 10 (53): 329-39).

The use of monoclonal antibodies for cancer treatment has had substantial success. For example, monoclonal antibodies are suitable for target-directed addressing of tumor tissue and tumor cells. Antibody-drug conjugates have become a novel and powerful option for the treatment of lymphomas and solid cancers, and immunomodulatory antibodies also have recently had considerable success in clinical trials. The development of therapeutic antibodies is based on deep understanding of cancer serology, protein engineering technology and the action thereof, mechanisms of resistance, and interactions between immune systems and cancer cells.

Antigens which are expressed on the surface of human cancer cells are defined as a broad range of targets which are over-expressed compared to normal tissues, mutated and selectively expressed. The key challenge is to identify antigens suitable for antibody-based therapies. These therapeutic agents mediate changes in antigen or receptor function (i.e., function as a stimulant or an antagonist), regulate the immune system through Fc and T cell activation, and exhibit efficacy through the delivery of specific drugs that bind to antibodies targeting specific antigens. Molecular techniques that can alter antibody pharmacokinetics, action function, size and immune stimulation are emerging as key factors in the development of novel antibody-based therapies. Evidences from clinical trials of therapeutic antibodies in cancer patients highlights the importance of approaches for selecting optimized antibodies, including affinity and binding of target antigens and antibodies, selection of an antibody structure, and therapeutic approaches (signaling blockade or immune function).

Human TROP2 (tumor-associated calcium signal transducer 2, TACSTD2) is a 35-46 kDa transmembrane calcium signal transducer glycoprotein having 323 amino acids (274 extracellular domains, 23 transmembrane domains, and 26 intracellular domains), and belongs to the epithelial cell adhesion molecule (EpCAM) family, which is an important component in cell signaling, proliferation, and differentiation. The expression of TROP2 is associated with tumor induction or tumor suppression in various carcinomas, but TROP2 is known to generally act as a tumor inducer.

The cleavage of the region between R87 and T88 from among 274 extracellular regions induces the rearrangement of the TROP2 structure, resulting in changes in biological activity. Although this cleavage does not occur in normal tissues, TROP2 cleavage occurs in most carcinomas, including skin cancer, ovarian cancer, colon cancer, and breast cancer, and thus, TROP2 acts as a tumor inducer.

In certain aspects, the present disclosure provides antibody-linker-drug (e.g., toxin) systems which, by applying a linker optionally comprising a self-immolative group, is: more stable in circulation (e.g., in plasma), enables a drug to be easily released in cancer cells to maximize efficacy, and enables a drug and/or toxin to stably reach a target cell.

Therefore, in certain embodiments, the present disclosure provides antibody-drug conjugates targeting human TROP2 (tumor-associated calcium signal transducer 2, TACSTD2), as well as pharmaceutically acceptable salts thereof.

In another aspect, the present disclosure provides pharmaceutical compositions comprising the antibody-drug conjugates disclosed herein or a pharmaceutically acceptable salt thereof.

In certain aspects, the present disclosure provides a conjugate represented by General Formula I below or a pharmaceutically acceptable salt or solvate thereof:

Ab-[L-(B)₁]ₘ [General Formula I]
wherein,
Ab is an anti-TROP2 (tumor-associated calcium signal transducer 2 aka TACSTD2) antibody or antigen-binding fragment thereof comprising a heavy chain variable region and a light chain variable region wherein:
   the heavy chain variable region comprises a heavy chain CDR1 comprising the amino acid sequence of SEQ ID NO: 2, a heavy chain CDR2 comprising the amino acid sequence of SEQ ID NO: 4, a and heavy chain CDR3 comprising the amino acid sequence of SEQ ID NO: 6; and
   the light chain variable region comprises a light chain CDR1 comprising the amino acid sequence of SEQ ID NO: 9, a light chain CDR2 comprising the amino acid sequence of SEQ ID NO: 11, and a light chain CDR3 comprising the amino acid sequence of SEQ ID NO: 13;
L is a linker;
B is an active agent moiety, and

In certain embodiments, the antibody or antigen-binding fragment thereof may include a heavy chain variable region comprising: the amino acid sequence of SEQ ID NO: 15 or 20; or a sequence with at least 80% sequence identity to the amino acid sequence of SEQ ID NO: 15 or 20.

In certain embodiments, the antibody or antigen-binding fragment thereof may include a heavy chain variable region comprising: the amino acid sequence of SEQ ID NO: 15 or 20; or a sequence with at least 80% sequence identity, at least 85% sequence identity, at least 90% sequence identity, or at least 95% sequence identity to the amino acid sequence of SEQ ID NO: 15 or 20 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6.

In certain embodiments, the antibody or antigen-binding fragment thereof may include a heavy chain variable region consisting of: the amino acid sequence of SEQ ID NO: 15 or 20; or a sequence with at least 80% sequence identity, at least 85% sequence identity, at least 90% sequence identity, or at least 95% sequence identity to the amino acid sequence of SEQ ID NO: 15 or 20 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6.

In certain embodiments, the antibody or antigen-binding fragment thereof may include a heavy chain variable region comprising: the amino acid sequence of SEQ ID NO: 15 or 20; a sequence with at least 90% sequence identity to the amino acid sequence of SEQ ID NO: 15 or 20; or a sequence with at least 95% sequence identity to the amino acid sequence of SEQ ID NO: 15 or 20 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6.

In certain embodiments, the antibody or antigen-binding fragment thereof may include a heavy chain variable region consisting: the amino acid sequence of SEQ ID NO: 15 or 20; a sequence with at least 90% sequence identity to the amino acid sequence of SEQ ID NO: 15 or 20; or a sequence with at least 95% sequence identity to the amino acid sequence of SEQ ID NO: 15 or 20 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6.

In certain embodiments, the antibody or antigen-binding fragment thereof may include a light chain variable region comprising: the amino acid sequence of SEQ ID NO: 16; or a sequence with at least 80% sequence identity to the amino acid sequence of SEQ ID NO: 16.

In certain embodiments, the antibody or antigen-binding fragment thereof may include a light chain variable region comprising: the amino acid sequence of SEQ ID NO: 16; or a sequence with at least 80% sequence identity, at least 85% sequence identity, at least 90% sequence identity, or at least 95% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13.

In certain embodiments, the antibody or antigen-binding fragment thereof may include a light chain variable region consisting of: the amino acid sequence of SEQ ID NO: 16; or a sequence with at least 80% sequence identity, at least 85% sequence identity, at least 90% sequence identity, or at least 95% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13.

In certain embodiments, the antibody or antigen-binding fragment thereof may include a light chain variable region comprising: the amino acid sequence of SEQ ID NO: 16; a sequence with at least 90% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13; or a sequence with at least 95% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13.

In certain embodiments, the antibody or antigen-binding fragment thereof may include a light chain variable region consisting of: the amino acid sequence of SEQ ID NO: 16; a sequence with at least 90% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13; or a sequence with at least 95% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13.

In certain embodiments, the antibody or antigen-binding fragment thereof comprises: a heavy chain variable region comprising the amino acid sequence of SEQ ID NO: 15 and a light chain variable region that comprising the amino acid sequences of SEQ ID NO: 16; or a heavy chain variable region comprising the amino acid sequence with at least 80% sequence identity to the amino acid sequence of SEQ ID NO: 15 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6 and a light chain variable region comprising the amino acid sequence with at least 80% sequence identity to the amino acid sequences of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13, and specifically binds to human TROP2.

In certain embodiments, the antibody or antigen-binding fragment thereof consists of: a heavy chain variable region comprising the amino acid sequence of SEQ ID NO: 15 and a light chain variable region that comprising the amino acid sequences of SEQ ID NO: 16; or a heavy chain variable region comprising the amino acid sequence with at least 80% sequence identity to the amino acid sequence of SEQ ID NO: 15 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6 and a light chain variable region consisting of the amino acid sequence with at least 80% sequence identity to the amino acid sequences of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13, and specifically binds to human TROP2.

In certain embodiments, the antibody or antigen-binding fragment thereof comprises: a heavy chain variable region comprising the amino acid sequence of SEQ ID NO: 15 and a light chain variable region comprising the amino acid sequences of SEQ ID NO: 16; or a heavy chain variable region comprising the amino acid sequence with at least 80% sequence identity, at least 85% sequence identity, at least 90% sequence identity, or at least 95% sequence identity to the amino acid sequence of SEQ ID NO: 15 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6 and a light chain variable region comprising amino acid sequences with at least 80% sequence identity, at least 85% sequence identity, at least 90% sequence identity, or at least 95% sequence identity to the amino acid sequences of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13, and specifically binds to human TROP2.

In certain embodiments, the antibody or antigen-binding fragment thereof comprises: a heavy chain variable region consisting of the amino acid sequence of SEQ ID NO: 15 and a light chain variable region comprising the amino acid sequences of SEQ ID NO: 16; or a heavy chain variable region comprising the amino acid sequence with at least 80% sequence identity, at least 85% sequence identity, at least 90% sequence identity, or at least 95% sequence identity to the amino acid sequence of SEQ ID NO: 15 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6 and a light chain variable region consisting of amino acid sequences with at least 80% sequence identity, at least 85% sequence identity, at least 90% sequence identity, or at least 95% sequence identity to the amino acid sequences of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13, and specifically binds to human TROP2.

In certain embodiments, the antibody or antigen-binding fragment thereof comprises: a heavy chain variable region comprising the amino acid sequence of SEQ ID NO: 15 and a light chain variable region comprising the amino acid sequences of SEQ ID NO: 16; or a heavy chain variable region comprising the amino acid sequence with at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or at least 99.5% sequence identity to the amino acid sequence of SEQ ID NO: 15 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6 and a light chain variable region comprising amino acid sequences with at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or at least 99.5% sequence identity to the amino acid sequences of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13, and specifically binds to human TROP2.

In certain embodiments, the antibody or antigen-binding fragment thereof comprises: a heavy chain variable region comprising the amino acid sequence of SEQ ID NO: 15 and a light chain variable region consisting of the amino acid sequences of SEQ ID NO: 16; or a heavy chain variable region comprising the amino acid sequence with at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or at least 99.5% sequence identity to the amino acid sequence of SEQ ID NO: 15 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6 and a light chain variable region consisting of amino acid sequences with at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or at least 99.5% sequence identity to the amino acid sequences of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13, and specifically binds to human TROP2.

In certain embodiments, the antibody is a humanized antibody.

In certain embodiments, the humanized antibody comprises: (a) (i) a variable heavy chain framework region from a heavy chain of a human antibody or from a human consensus framework; and (ii) a variable light chain framework region from a light chain of a human antibody or from a human consensus framework. In some embodiments, at least one amino acid in the variable domain framework region from the heavy chain is substituted with the corresponding amino acid from a heavy chain of a mouse antibody or mouse consensus framework; or the variable domain framework region from the light chain is substituted with the corresponding amino acid from a light chain of a mouse antibody or mouse consensus framework. In some embodiments, the amino acid substitution of the heavy chain is selected from the following amino acid substitutions:
substitution of Y (human) with F (mouse) at position 27;
substitution of T (human) with S (mouse) at position 30;
substitution of V (human) with L (mouse) at position 37;
substitution of M (human) with I (mouse) at position 48;
substitution of G (human) with A (mouse) at position 49;
substitution of I (human) with of L (mouse) at position 70; and
substitution R (human) with of V (mouse) at position 72, and
(ii) the amino acid substitution of the light chain is substitution of Y (human) with of S (mouse) at position 49.

In certain embodiments, the amino acid substitution of the heavy chain may further include the following amino acid substitution:
substitution of R (human) with K (mouse) at position 67; and/or
substitution of V (human) with A (mouse) at position 68.

In certain embodiments, the amino acid substitution of the light chain may further include the following amino acid substitution:
substitution of S (human) with Y (mouse) at position 67;
substitution of I (human) with T (mouse) at position 2 and substitution of S (human) with Y (mouse) at position 67;
substitution of T (human) with R (mouse) at position 22 and substitution of S (human) with Y (mouse) at position 67;
substitution of K (human) with E (mouse) at position 42 and substitution of S (human) with Y (mouse) at position 67;
substitution of G (human) with S (mouse) at position 64 and substitution of S (human) with Y (mouse) at position 67;
substitution of S (human) with Y (mouse) at position 67 and substitution of T (human) with V (mouse) at position 72; or
substitution of S (human) with Y (mouse) at position 67 and substitution of T (human) with D (mouse) at position 85.

In certain embodiments, the humanized antibody comprises: (a) (i) a variable heavy chain framework region from a heavy chain of a human antibody or from a human consensus framework, wherein the variable heavy chain framework region comprises one or more of the following amino acid sequence changes: Y27F, T30S, V37L, M48I, G49A, 170L, and R72V; and (ii) a variable light chain framework region from a light chain of a human antibody or from a human consensus framework, wherein the variable light chain framework region comprises the following amino acid sequence change: Y49S.

The amino acid substitution may increase affinity, enhance the stability of antibodies while maintaining their antigen binding activity. Stabilization of therapeutic antibodies can result in improved serum half-life, lower dosage requirements, reduced side-effects, and improved shelf-life. The chimeric CH-2EF and CH-2G10 antibodies were built first, followed by the early humanized versions: Hu-2EF-4, Hu-2G10-1, etc. These antibodies may show negative characteristics such as chemical instability, with formation of aggregates and loss of solubility (for the CH-2EF antibody), and loss of affinity for Trop-2. The amino acid substitution may create humanized monoclonal antibodies with high affinity, including Hu-2EF-7, H, Hu-2G10-5 and Hu-2G10-6, which are not recognized by anti-mouse antibodies, specifically directed against distinct regions and various forms of post-translational modification of the extracellular domain of Trop2.

In certain embodiments, human VH sequences homologous to the VH frameworks disclosed herein were searched for within the GenBank database, and the VH sequence encoded by the human cDNA with NCBI Accession number: X65888.1 was chosen as an acceptor for humanization (X65888.1-VH). In certain embodiments, the CDR sequences of VH disclosed herein were first transferred to the corresponding positions of X65888.1-VH. In certain embodiments, at framework positions 27, 30, 37, 48, 49, 67, 68, 70 and 72 (numbering from the first amino acid of the mature form), where the three-dimensional model of the variable regions suggested significant contact with the CDRs, the human amino acid residues were substituted with the corresponding mouse residues.

While Lys at position 67 and Ala at position 68 in mouse 2G10 VH, both of which are located closely to the CDRs, were predicted to be important for the formation of the CDR structure, amino acid residues in the human acceptor X65888.1-VH are Arg at position 67 and Val at position 68, which are similar to the corresponding mouse residues for molecular property and structure. Therefore, in order to further reduce potential immunogenicity, a second humanized VH (VH2) was designed, in which the amino acid residues of the human acceptor are maintained at position 67 and 68, i.e. Arg and Val, respectively.

Based on the GenBank homology search with the VL framework sequences, the human VK region encoded by the cDNA with NCBI Accession number: AY043146.1 was chosen as an acceptor for humanization. CDR sequences of VL were first transferred to the corresponding positions of AY043146.1 VL. Next, at positions 49 and 67, where the three-dimensional model of the variable regions indicated significant contact with the CDRs, the human amino acid residues were substituted with the corresponding residues of the mouse VL. Although Tyr at position 67 in mouse VL was predicted to be important for the formation of the CDR structure, further analysis of the three-dimensional model of the 2G10 variable regions suggested a possibility that this Tyr at position 67 could be replaced with Ser, a residue located at the corresponding position in the human acceptor AY043146.1 VL, without affecting antigen binding. Therefore in order to further reduce potential immunogenicity, a second humanized VL was designed, derived from VLI, in which the Ser at position 67 in the human acceptor sequence is maintained.

In certain embodiments, the antibody or antigen-binding fragment thereof may be selected from the group consisting of a monoclonal antibody, a domain antibody (dAb), a single chain antibody (scAb), a Fab fragment, a Fab fragment, a F(ab [symbol] 2 fragment, an scFab fragment, an Fv fragment, a dsFv fragment, a single chain variable fragment (scFv), an scFv-Fc fragment, a single domain heavy chain antibody, a single domain light chain antibody, a variant antibody, a multimeric antibody, a minibody, a diabody, a bispecific antibody, and a multispecific antibody.

The linker described herein may be cleavable, non-cleavable and hydrophilic or hydrophobic.

In certain embodiments, the cleavable linker is cleavable under intracellular or extracellular conditions, by which an active agent is released from an antibody construct-active agent conjugate in the intracellular environment.

The cleavable linker can be cleaved by a cleaving agent present in an intracellular environment (e.g., lysosomes, endosomes, or caveolae). The cleavable linker may be, for example, a peptidyl linker that is cleaved by an intracellular peptidase or protease enzyme, including, but not being limited to, a lysosomal or endosomal protease. Generally, the peptidyl linker has a length of at least two amino acids or a length of at least three amino acids. Cleaving agents may include cathepsin B, cathepsin D, and plasmin, all of which are known to hydrolyze dipeptide drug derivatives to release an active drug in target cells (e.g., see Dubowchik and Walker, 1999, Pharm. Therapeutics 83:67-123). The most common are peptidyl linkers cleavable by enzymes present in antigen-expressing cells. For example, peptidyl linkers cleavable by thiol-dependent protease cathepsin-B, which is highly expressed in cancer tissue, may be used (e.g., a Phe-Leu or Gly-Phe-Leu-Gly linker). Other examples of these linkers are described in, for example, U.S. Pat. No. 6,214,345. In addition, the peptidyl linker cleavable by an intracellular protease may be, for example, a Val-Cit linker, a Phe-Lys linker (e.g., see U.S. Pat. No. 6,214,345, which describes the synthesis of doxorubicin using a Val-Cit linker), or a Val-Ala linker. The Val-Cit linker or the Val-Ala linker may contain a pentafluorophenyl group, and may contain a succinimide group or a maleimide group. Alternatively, the Val-Cit linker or the Val-Ala linker may contain a pentafluorophenyl group, may contain a 4-aminobenzoic acid (PABA) group and a maleimide group, and may contain a PABA group and a succinimide group.

In addition, a cleavable linker may be easily hydrolyzed in a pH-sensitive manner, i.e., at certain pH values. Generally, the pH-sensitive linker may be hydrolyzed under acidic conditions. For example, acid-labile linkers that can be hydrolyzed in lysosomes (e.g., hydrazone, semicarbazone, thiosemicarbazone, cis-aconic amides, orthoesters, acetals, and ketals) may be used (e.g., see: U.S. Pat. Nos. 5,122,368, 5,824,805, and 5,622,929; and Dubowchik and Walker, 1999, Pharm. Therapeutics 83:67-123; and Neville et al., 1989, Biol. Chem. 264:14653-14661). These linkers are relatively stable under neutral pH conditions, such as in blood, but are unstable at pH 5.5, which is the approximate pH of lysosomes, or less than pH 5.0. Examples of hydrolysable linkers include thioether linkers (e.g., thioethers attached to a therapeutic agent via an acylhydrazone bond) (e.g., see U.S. Pat. No. 5,622,929).

Also, the linker is cleavable under reducing conditions (e.g., a disulfide linker). For example, various disulfide linkers including N-succinimidyl-5-acetylthioacetate (SATA), N-succinimidyl-3-(2-pyridyldithio) propionate (SPDP), N-succinimidyl-3-(2-pyridyldithio) butyrate (SPDB), and N-succinimidyl-oxycarbonyl-alpha-methyl-alpha-(2-pyridyl-thio) toluene)-(SMPT), and those that can be formed using SPDB and SMPT (e.g., see: Thorpe et al., 1987, Cancer Res. 47:5924-5931; and U.S. Pat. No. 4,880,935).

In addition, the linker may be a malonate linker (Johnson et al., 1995, Anticancer Res. 15:1387-93), a maleimidobenzoyl linker (Lau et al., 1995, Bioorg-Med-Chem. 3 (10): 1299-1304), a 3-N-amide analogue (Lau et al., 1995, Bioorg-Med-Chem. 3 (10): 1305-12), a β-glucuronide linker (Jeffery et al., 2006, Bioconjug Chem. 17 (3): 832-40), or a β-galactoside linker (Kolodych et al., 2017, Eur J Med Chem. December 15; 142:376-382).

The non-cleavable linker may be a maleimidocaproyl linker. The maleimidocaproyl linker may include N-maleimidomethylcyclohexane-1-carboxylate. The maleimidocaproyl linker may contain a succinimide group. The maleimidocaproyl linker may contain a pentafluorophenyl group. The linker may be a combination of a maleimide group and one or more polyethylene glycol molecules. The linker may be a combination of a maleimidocaproyl group and one or more polyethylene glycol molecules. The linker may be a maleimide-PEG4 linker. The linker may be a combination of a maleimidocaproyl linker containing a succinimide group and one or more polyethylene glycol molecules. The linker may be a combination of a pentafluorophenyl group and a maleimidocaproyl linker containing one or more polyethylene glycol molecules. The linker may contain a maleimide linked to a polyethylene glycol molecule, wherein the polyethylene glycol allows for more linker flexibility or allows longer linkers to be used. The linker may be a (maleimidocaproyl)-(valine-citrulline)-(para-aminobenzyloxycarbonyl) linker.

In certain embodiments, the linker may be a cleavable linker.

In certain embodiments, the linker may be a protease cleavable linker, an acid-cleavable linker, a disulfide linker, a self-immolative linker or a self-stabilizing linker, a malonate linker, a maleimidobenzoyl linker, a 3 [symbol] N-amide analogue, a β-glucuronide linker, or a β-galactoside linker.

In certain embodiments, the protease cleavable linker may include a thiol-reactive spacer or a dipeptide, and more specifically, the protease cleavable linker may include a thiol-reactive maleimidocaproyl spacer, a valine-citrulline dipeptide, or a p-amino-benzyloxycarbonyl spacer.

In certain embodiments, the acid-cleavable linker may be a hydrazine linker or a quaternary ammonium linker.

Exemplary antibody drug conjugates are disclosed in U.S. Pat. Nos. 10,583,197, 9,993,568, 9,951,072, 9,919,057, 9,669,107, 11,413,353, 11,173,214, 11,167,040, 10,980,890, 10,583,197, 10,383,949, 10,273,235, 10,183,997, and 10,118,965, the contents of each of which is fully incorporated by reference herein.

In certain embodiments, the linker may have a structure of General Formula II.

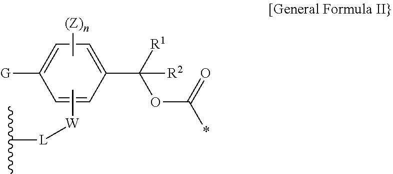

wherein: in General Formula II,
a wave symbol is a site that is linked to an antibody or antigen-binding fragment thereof that specifically binds to human TROP2, and
* is a site that is linked to an active agent;
G is a glucuronic acid moiety or

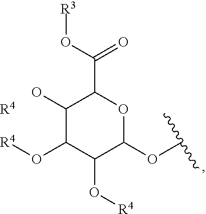

R³ is hydrogen or a carboxyl-protecting group, and each of R⁴ is independently hydrogen or a hydroxyl-protecting group;
R¹ and R² are each independently hydrogen, C₁₋₈ alkyl, or C₃₋₈ cycloalkyl;
W is —C(O)—, —C(O)NR [symbol] , —C(O)O—, —SO2NR [symbol] , —P(O)R [symbol] R [symbol] -SONR [symbol] , or —PO2NR [symbol] , wherein C, S, or P is directly bonded to a phenyl ring, and R [symbol] nd R [symbol] re each independently hydrogen, C₁₋₈ alkyl, C₃₋₈ cycloalkyl, C₁₋₈ alkoxy, C₁₋₈ alkylthio, mono- or di-C₁₋₈ alkylamino, C₃₋₂₀ heteroaryl, or C₆₋₂₀ aryl;
Z is independently hydrogen, C₁₋₈ alkyl, halogen, cyano, or nitro;
n is 0 to 3; and
L is any one selected from A) or B) below:
A) C₁₋₅₀ alkylene or 1- to 50-membered heteroalkylene, satisfying at least one of the following:
(i) L includes one or more unsaturated bonds;
(ii) two atoms in L are substituted with a divalent substituent, which completes a heteroarylene;
(iii) L is a 1- to 50-membered heteroalkylene;
(iv) the alkylene is substituted with at least one C₁₋₂₀ alkyl; and
B) comprises at least one isoprenyl derivative unit of General Formula III below which can be recognized by an isoprenoid transferase:

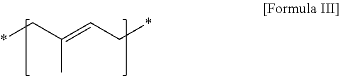

In certain embodiments, the conjugate may have a structure of General Formula IIa.
[General Formula IIa]

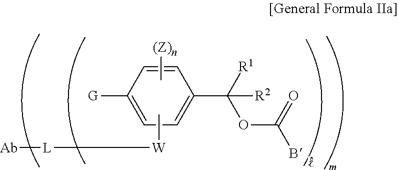

wherein
each B is an active agent;

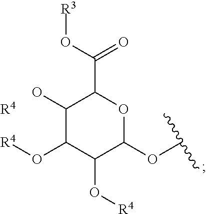

each G is independently a glucuronic acid moiety or
R³ is hydrogen or a carboxyl-protecting group;
each R⁴ is independently hydrogen or a hydroxyl-protecting group;
R¹ and R² are each independently hydrogen, C₁₋₈ alkyl, or C₃₋₈ cycloalkyl;
W is —C(O)—, —C(O)NR [symbol] , —C(O)O—, —SO₂NR [symbol] , —P(O)R [symbol] R [symbol] SONR [symbol] or —PO₂NR [symbol] , wherein the C, S, or P is directly bonded to the phenyl ring of Formula IIa;
R [symbol] nd R [symbol] re each independently hydrogen, C₁₋₈ alkyl, C₃₋₈ cycloalkyl, C₁₋₈ alkoxy, C₁₋₈ alkylthio, mono- or di-C₁₋₈ alkylamino, C₃₋₂₀ heteroaryl, or C₆₋₂₀ aryl;
each Z is independently hydrogen, C₁₋₈ alkyl, halogen, cyano, or nitro;
n is 0, 1, 2, or 3;
L comprises:
   A) C₁₋₅₀ alkylene or C₁₋₅₀ heteroalkylene and comprises
      (i) one or more unsaturated bonds
      (ii) a heteroarylene; or
      (iii) is substituted with at least one C₁₋₂₀ alkyl; or
   B) at least one isoprenyl group having a structure represented by General Formula III:

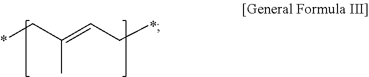

and
   1 and m are each independently 1 to 20.

The present disclosure also provides a conjugate represented by General Formula IIa below or a pharmaceutically acceptable salt or solvate thereof:

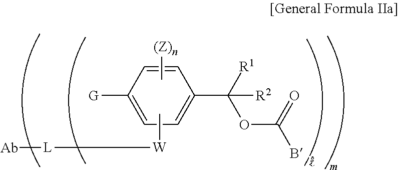

wherein
Ab is an anti-TROP2 (tumor-associated calcium signal transducer 2 aka TACSTD2) antibody or antigen-binding fragment thereof;
each B is an active agent;

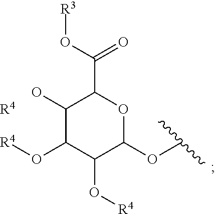

each G is independently a glucuronic acid moiety or
R³ is hydrogen or a carboxyl-protecting group;
each R⁴ is independently hydrogen or a hydroxyl-protecting group;
R¹ and R² are each independently hydrogen, C₁₋₈ alkyl, or C₃₋₈ cycloalkyl;
W is —C(O)—, —C(O)NR [symbol] —C(O)O—, —SO₂NR [symbol] —P(O)R [symbol] R [symbol] —SONR [symbol] or —PO₂NR [symbol] wherein the C, S, or P is directly bonded to the phenyl ring of Formula IIa;
R [symbol] nd R [symbol] re each independently hydrogen, C₁₋₈ alkyl, C₃₋₈ cycloalkyl, C₁₋₈ alkoxy, C₁₋₈ alkylthio, mono- or di-C₁₋₈ alkylamino, C₃₋₂₀ heteroaryl, or C₆₋₂₀ aryl;
each Z is independently hydrogen, C₁₋₈ alkyl, halogen, cyano, or nitro;
n is 0, 1, 2, or 3;
L comprises:
   A) C₁₋₅₀ alkylene or C₁₋₅₀ heteroalkylene and comprises
      (i) one or more unsaturated bonds
      (ii) a heteroarylene; or
      (iii) is substituted with at least one C₁₋₂₀ alkyl; or
   B) at least one isoprenyl group having a structure represented by General Formula III:

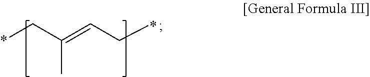

and
1 and m are each independently 1 to 20.

Additional conjugates related to this structure, as well as detailed preparation methods, are disclosed in U.S. Pat. Nos. 9,919,057, 9,993,568, 10,980,890 and 11,413,353; the contents of each of which is fully incorporated by reference herein.

In certain embodiments, the antibody or antigen-binding fragment thereof comprises a heavy chain variable region and a light chain variable region, wherein:
the heavy chain variable region comprises a heavy chain CDR1 comprising the amino acid sequence of SEQ ID NO: 2, a heavy chain CDR2 comprising the amino acid sequence of SEQ ID NO: 4, a and heavy chain CDR3 comprising the amino acid sequence of SEQ ID NO: 6; and
the light chain variable region comprises a light chain CDR1 comprising the amino acid sequence of SEQ ID NO: 9, a light chain CDR2 comprising the amino acid sequence of SEQ ID NO: 11, and a light chain CDR3 comprising the amino acid sequence of SEQ ID NO: 13.

In certain embodiments, the antibody or antigen-binding fragment thereof comprises a heavy chain variable region comprising:
the amino acid sequence of SEQ ID NO: 15 or 20;
a sequence having at least 90% sequence identity to the amino acid sequence of SEQ ID NO: 15 or 20 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6; or
a sequence having at least 95% sequence identity to the amino acid sequence of SEQ ID NO: 15 or 20 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6.

In certain embodiments, the antibody or antigen-binding fragment thereof comprises a light chain variable region comprising:
the amino acid sequence of SEQ ID NO: 16;
a sequence having at least 90% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13; or
a sequence having at least 95% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13.

In certain embodiments, the antibody or antigen-binding fragment thereof comprises a heavy chain variable region comprising the amino acid sequence of SEQ ID NO: 15 and a light chain variable region comprising the amino acid sequences of SEQ ID NO: 16.

In certain embodiments, the antibody is a humanized antibody. In certain embodiments, the humanized antibody comprises:
(i) a variable heavy chain framework region from a heavy chain of a human antibody or from a human consensus framework, wherein the variable heavy chain framework region comprises one or more of the following amino acid sequence changes: Y27F, T30S, V37L, M48I, G49A, 170L, and R72V; and
(ii) a variable light chain framework region from a light chain of a human antibody or from a human consensus framework, wherein the variable light chain framework region comprises the following amino acid sequence change: Y49S.

In certain embodiments, the antibody or antigen-binding fragment thereof is selected from a monoclonal antibody, a domain antibody (dAb), a single chain antibody (scAb), a Fab fragment, a Fab [symbol] ragment, a F(ab [symbol] 2 fragment, an scFab fragment, an Fv fragment, a dsFv fragment, a single chain variable fragment (scFv), an scFv-Fc fragment, a single domain heavy chain antibody, a single domain light chain antibody, a variant antibody, a multimeric antibody, a minibody, a diabody, a bispecific antibody, and a multispecific antibody.

In certain preferred embodiments, each G is

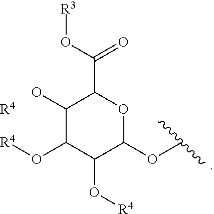

In certain embodiments, R¹ and R² are each hydrogen.

In certain embodiments, R³ is hydrogen.

In certain embodiments, each R⁴ is a hydroxyl-protecting group.

In certain embodiments, n is 0.

In certain preferred embodiments, each W is —C(O)NR′—, further wherein the C is directly bonded to the phenyl ring of Formula IIa, and NR′ is bonded to L.

In certain embodiments, each R⁴ is independently hydrogen.

In certain embodiments, R¹ and R² are each hydrogen;
n is 0; and
each W is —C(O)NR′—, C is directly bonded to the phenyl ring of Formula IIa, and R′ is hydrogen, C₁₋₈ alkyl, C₃₋₈ cycloalkyl, C₁₋₈ alkoxy, C₁₋₈ alkylthio, mono- or di-C₁₋₈ alkylamino, C₃₋₂₀ heteroaryl, or C₆₋₂₀ aryl, wherein NR′ is bonded to L.

In certain embodiments, L comprises a nitrogen-containing 1- to 50-membered heteroalkylene.

In certain embodiments, L comprises a hydrophilic amino acid. In certain embodiments, W comprises two or more atoms of the hydrophilic amino acid, and the nitrogen of W forms a peptide bond with a carbonyl of the hydrophilic amino acid.

In certain embodiments, L is a nitrogen-containing 1- to 50-membered heteroalkylene, the linker comprises two or more atoms of a hydrophilic amino acid, and the nitrogen forms a peptide bond with a carbonyl of the hydrophilic amino acid.

In certain embodiments, L is covalently bonded to the antibody by a thioether bond and the thioether bond comprises a sulfur atom of a cysteine of the antibody.

In certain embodiments, the antibody comprises an amino acid motif recognizable by an isoprenoid transferase at the C-terminus of the antibody, and the thioether bond comprises a sulfur atom of a cysteine of the amino acid motif.

In certain embodiments, the amino acid motif has a CYYX sequence, further wherein:
C is cysteine;
Y is an aliphatic amino acid;
X is selected from glutamine, glutamate, serine, cysteine, methionine, alanine, and leucine; and
the thioether bond comprises a sulfur atom of a cysteine of the amino acid motif.

In certain embodiments, the amino acid motif has a CYYX sequence further wherein:

Y is selected from alanine, isoleucine, leucine, methionine, and valine.

In certain preferred embodiments, the amino acid motif has a CVIM (SEQ ID NO: 24) or CVLL sequence (SEQ ID NO: 25).

In certain embodiments, at least one of 1 to 20 amino acids preceding the amino acid motif is glycine.

In certain preferred embodiments, L comprises the amino acid sequence of GGGGGGGCVIM at the C-terminus (SEQ ID NO: 22).

In certain embodiments, L comprises a C₁₋₅₀ heteroalkylene.

In certain preferred embodiments, L comprises an oxime. In certain embodiments, the oxygen atom of the oxime is on the side of L linked to W and the carbon atom of the oxime is on the side of L linked to Ab. In certain embodiments, the carbon atom of the oxime is on the side of L linked to W and the oxygen atom of the oxime is on the side of L linked to Ab.

In certain embodiments, L is a C₁₋₅₀ heteroalkylene containing an oxime,
the oxygen atom of the oxime is on the side of L linked to W,
the carbon atom of the oxime is on the side of L linked to Ab, or
the carbon atom of the oxime is on the side of L linked to W, and
the oxygen atom of the oxime is on the side of L linked to Ab.

In certain preferred embodiments, L comprises an oxime, and at least one isoprenyl unit covalently bonds the oxime to Ab (e.g., at least one isoprenyl unit directly or indirectly bonds the oxime to Ab).

In certain embodiments, L comprises a connecting unit represented by General Formula VIII or General Formula IX:

—(CH₂)ᵣ(V(CH₂)ₚ)_{q}— [General Formula VIII]

—(CH₂CH₂X)_{w}— [Formula IX]
V is a single bond, —O—, —S—, —NR²¹—, —C(O)NR²²—, —NR²³C(O)—, —NR²⁴SO₂—, or —SO₂NR²⁵—;
X is —O—, C₁₋₈ alkylene, or —NR²¹;
R²¹ to R²⁵ are each independently hydrogen, C₁₋₆ alkyl, C₁₋₆ alkyl C₆₋₂₀ aryl, or C₁₋₆ alkyl-C₃₋₂₀ heteroaryl;
r is 0 to 10;
p is 0 to 10;
q is 1 to 20; and
w is 1 to 20.

In certain embodiments, q is 1 to 10.

In certain embodiments, r is 1 or 2.

In certain embodiments, p is 1 or 2.

In certain embodiments, V is —O—.

In certain embodiments, q is 1 to 10;
r and p are each 1 or 2; and
V is —O—.

In certain embodiments, X is —O—.

In certain embodiments, w is 1 to 10.

In certain embodiments, wherein:
the X is —O—; and
w is 1 to 10.

In certain preferred embodiments, L comprises

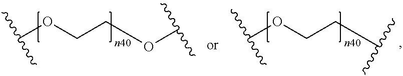

wherein n40 is 1-10, preferably at least 2.

In certain embodiments, L comprises an oxime, and at least one polyethylene glycol unit covalently bonds the oxime to an active agent.

In certain embodiments, L comprises a binding unit formed by a reaction between an alkyne and an azide or between an aldehyde or ketone group and hydrazine or hydroxylamine.

In certain embodiments, L further comprises a binding unit represented by General Formula IVa, IVb, IVc, IVd, or IVe below:
wherein

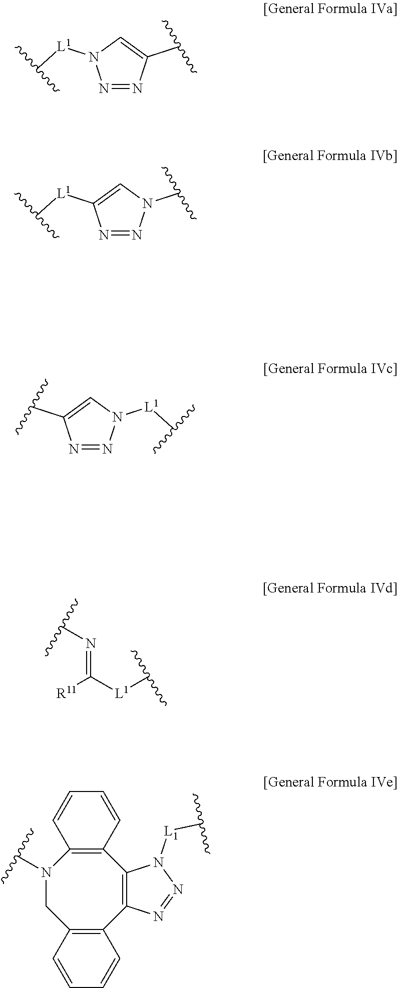

L¹ and L₂ are each independently a single bond or C₁₋₃₀ alkylene; and
R₁₁ is hydrogen or C₁₋₁₀ alkyl.

In certain embodiments, L₁ and L₂ are each independently a single bond, C₁₁ alkylene, or C₁₂ alkylene.

In certain embodiments, the isoprenoid transferase is farnesyl protein transferase (FTase) or geranylgeranyl transferase (GGTase).

In certain embodiments, L is branched and comprises:
i) a branching unit covalently coupled to the antibody by a primary linker;
ii) a first branch, in which a first active agent is covalently coupled to the branching unit by a secondary linker and a cleavage group; and
iii) a second branch, in which:
   a) a second active agent is covalently coupled to the branching unit by a secondary linker and a cleavage group; or
   b) a polyethylene glycol moiety is covalently coupled to the branching unit.

In certain embodiments, the branching unit has a structure represented by

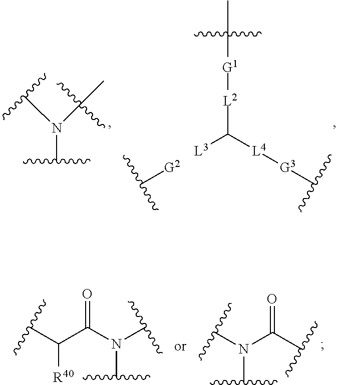

wherein
L², L³, and L⁴ are each independently a bond or —CₙH₂ₙ—,
n is 1 to 30;
G¹, G², and G³ each independently represent a bond,

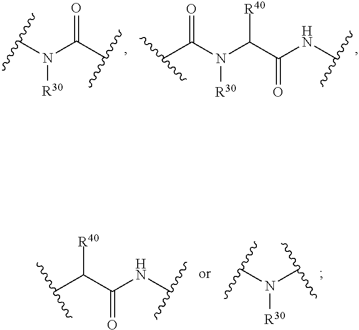

R³⁰ is hydrogen or C₁₋₃₀ alkyl;
R⁴⁰ is hydrogen or L⁵-COOR₆; and
L⁵ is a bond or —Cₙ [symbol] ;
n [symbol] s 1 to 10; and
R⁶ is hydrogen or C₁₋₃₀ alkyl.

In certain preferred embodiments, the cleavage group is cleavable in a target cell and is capable of releasing one or more active agents.

In certain embodiments, at least one branched linker is covalently coupled to Ab; and at least two active agents are covalently coupled to the branched linker. In certain embodiments, one branched linker is coupled to Ab. In certain embodiments, two branched linkers are coupled to Ab. In certain embodiments, three branched linkers are coupled to Ab. In certain embodiments, four branched linkers are coupled to Ab. In certain embodiments, each branched linker is coupled to two active agents. In certain embodiments, the conjugate comprises at least two different active agents. In certain embodiments, at least one branched linker is coupled to two different active agents.

In certain embodiments, the branching unit is a nitrogen atom. In other embodiments, the branching unit is an amide and the primary linker comprises the carbonyl of the amide. In yet other embodiments, the branching unit is an amide and the secondary linker comprises the carbonyl of the amide. In certain preferred embodiments, the branching unit is lysine.

In certain embodiments, the conjugate comprises a structure represented by:

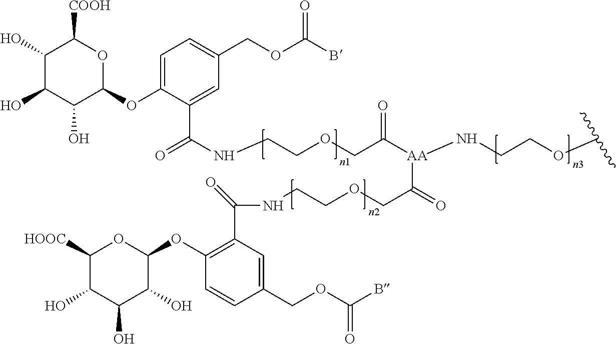

or a pharmaceutically acceptable salt thereof; wherein
B [symbol] and B [symbol] re each active agents;
n1 to n3 are each independently 0 to 30; and
AA is an amino acid group.

In certain embodiments, the conjugate comprises a structure represented by or a pharmaceutically acceptable salt thereof:

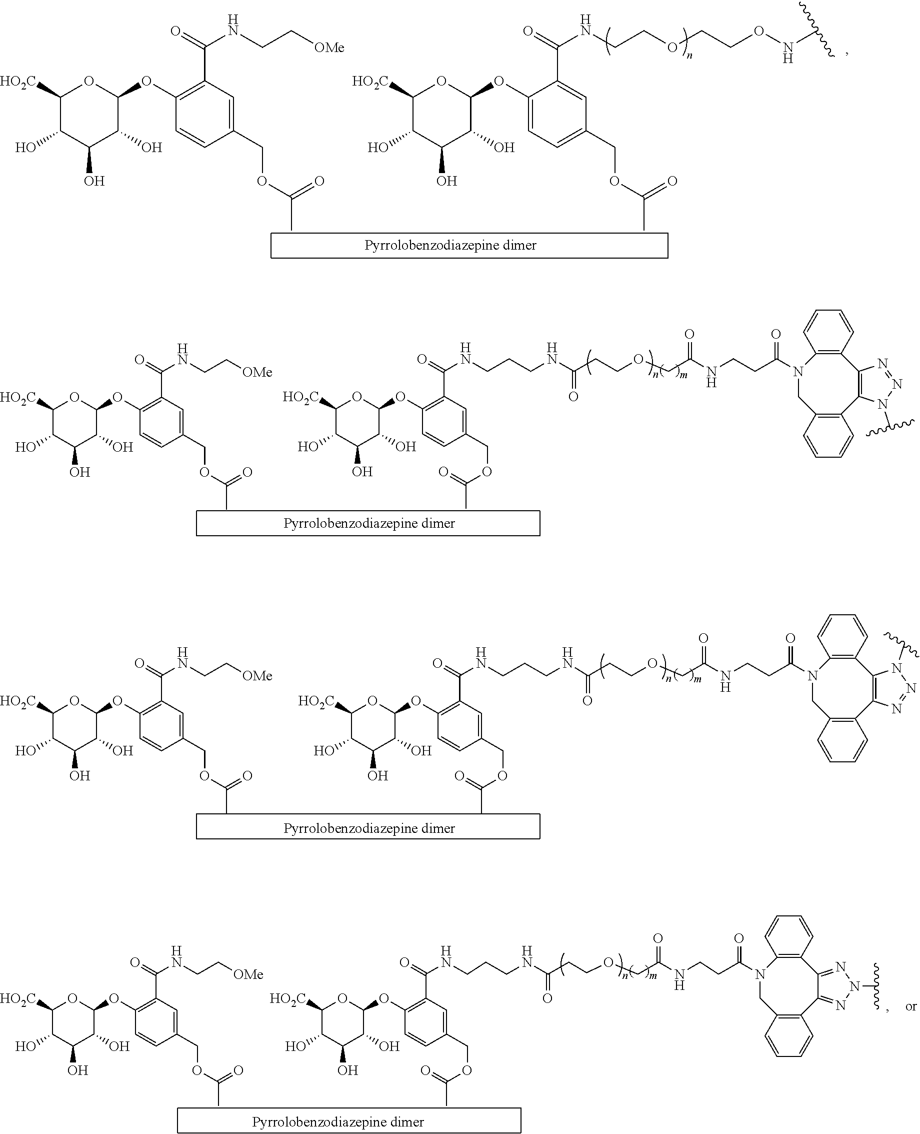

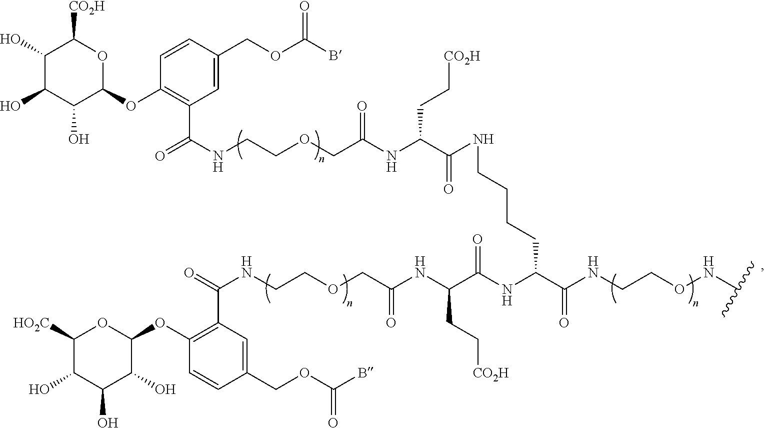

wherein B [symbol] nd B [symbol] efer to active agents which are identical to or different from each other, the pyrrolobenzodiazepine dimer, for example, a compound of formula III; and
m and n each independently refers to 0 to 30,
or a pharmaceutically acceptable salt thereof.

In certain embodiments, the conjugate comprises a structure represented by:

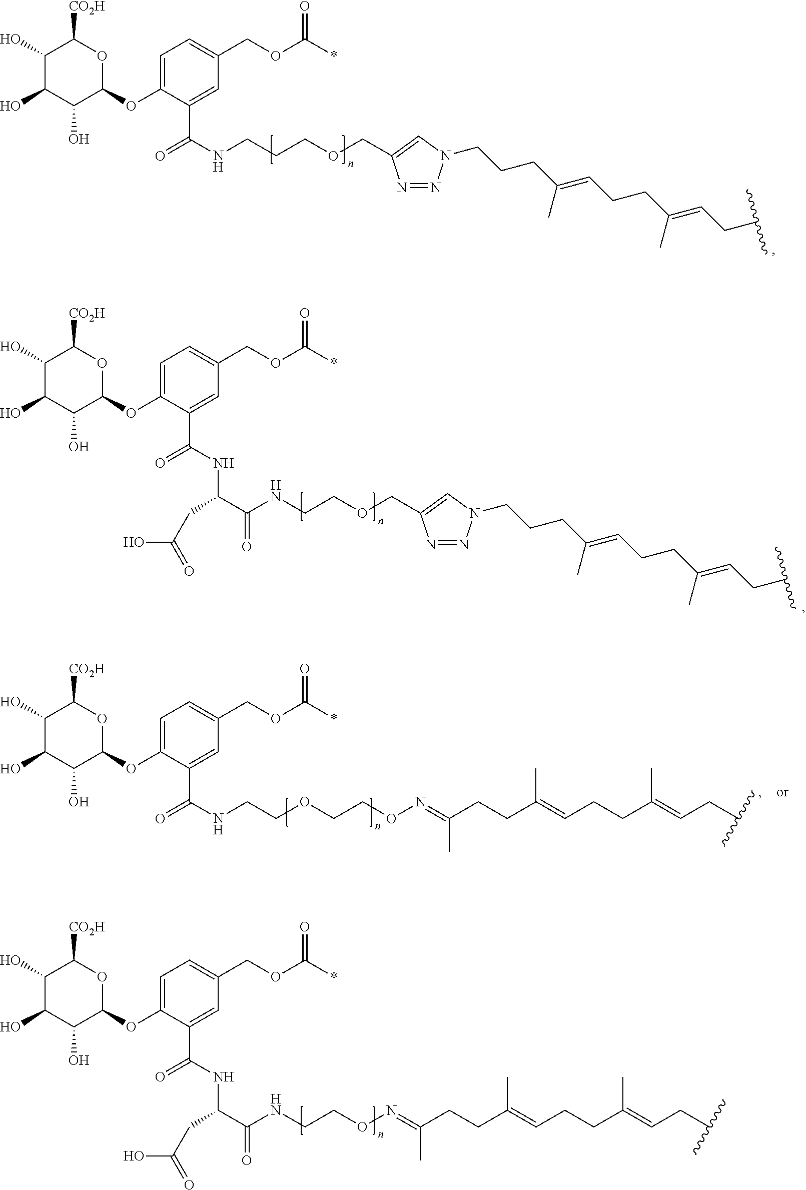

or a pharmaceutically acceptable salt thereof, wherein a wave symbol represents a binding site for the antibody construct, * represents a binding site for the active agent, and n is 0 to 20.

In certain embodiments, the first linker of the antibody-drug conjugate comprises an alkylene having 1 to 100 carbon atoms, preferably 1 to 50 carbon atoms, or alkylene includes at least one unsaturated bond, alkylene comprises at least one heteroarylene, the carbon atom of the alkylene is substituted with one or more heteroatoms selected from nitrogen (N), oxygen (O), and sulfur(S), or alkylene is further substituted with one or more alkyls having 1 to 20 carbon atom(s).

In certain embodiments, at least one carbon atom of the alkylene is substituted with nitrogen, the first linker includes at least two atoms of a hydrophilic amino acid, and nitrogen forms a peptide bond along with the main chain carbonyl of the hydrophilic amino acid. The hydrophilic amino acid may be, for example, arginine, aspartate, asparagine, glutamate, glutamine, histidine, lysine, ornithine, proline, serine, or threonine.

In certain embodiments, the branched linker of the antibody-active agent comprises an amino acid having a side chain with a moiety that carries a charge at neutral pH in aqueous solution, preferably arginine, aspartate, glutamate, lysine, or ornithine. The amino acid may be located anywhere on the branched linker. For example, the oxime of the branched linker may be covalently bonded to the polyethylene glycol unit of the branched linker. Alternatively or additionally, these amino acids may be present in the second linker, optionally within each second linker.

In certain embodiments, the conjugate comprises a structure represented by:

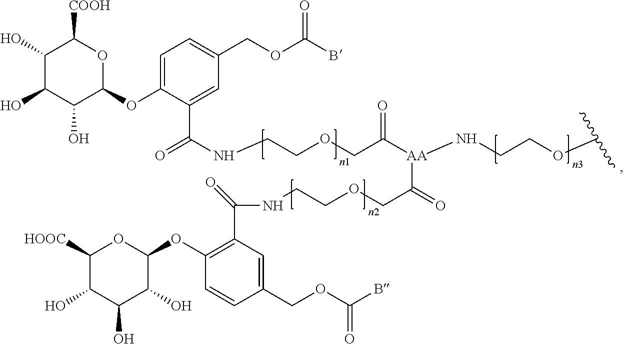

or a pharmaceutically acceptable salt thereof;
wherein B [symbol] nd B [symbol] re each an active agent;
n1 to n3 each independently refer to 0 to 30; and
AA refers to an amino acid group.

In certain embodiments, AA refers to an amino acid group in which one or more amino acids are bonded.

In certain embodiments, i) the linker includes a peptide sequence of multiple amino acids; and ii) at least two active agents are covalently bonded to the side chain of an amino acid.

In certain embodiments, the amino acid group is a group by main-chain linkage or side-chain linkage of 1 to 20 amino acids.

In certain embodiments, the amino acid group is a group by main-chain linkage or side-chain linkage of 1 to 20 arginine, aspartate, asparagine, glutamate, glutamine, histidine, lysine, ornithine, proline, serine or threonine residue(s).

In certain embodiments the amino acid group is a group by main-chain linkage or side-chain linkage of 1 to 20 arginine, aspartate, glutamate, lysine or ornithine residue(s).

In certain embodiments, the amino acid group includes 1 to 20 amino acids, e.g., at least one lysine.

In certain embodiments, the amino acid group comprises main-chain or side-chain linkage of lysine.

In certain embodiments, the conjugate comprises a structure represented by:

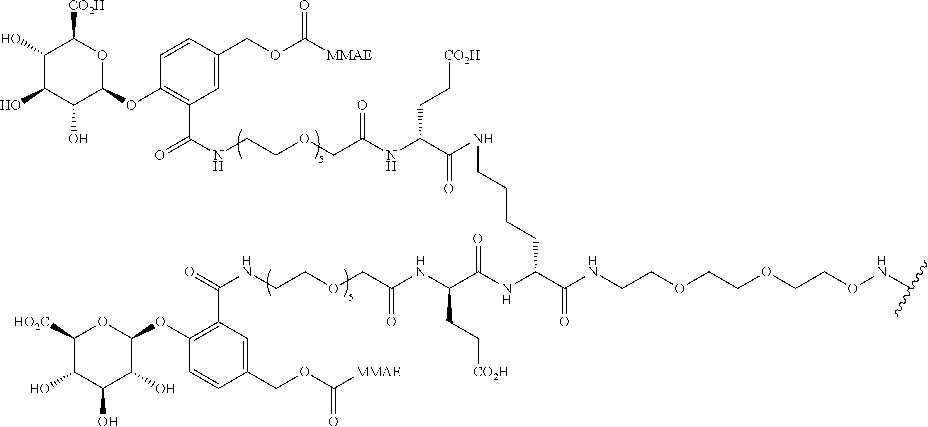

wherein, MMAE is monomethyl auristatin E

Additional conjugates related to this structure, as well as detailed preparation methods, are disclosed in U.S. Pat. Nos. 11,173,214 and 11,167,040; the contents of each of which is fully incorporated by reference herein.

In certain embodiments, the term “active agent moiety” as used herein refers to a compound that comprises an active agent and covalent bonds which bond the active agent to a linker or a part thereof.

In the present disclosure, the active agent moiety may be directly bonded to the linker, and one or more, particularly, two or more, three or more, or four or more active agent moieties may be directly bonded to the linker.

In certain embodiments, active agents are each independently selected from a chemotherapeutic agent and a toxin.

In addition, the active agent may be an immunomodulatory compound, an anticancer agent, an anti-viral agent, an antibacterial agent, an antifungal agent, an antiparasitic agent, or a combination thereof, and may be selected for use from active agents listed below:
(a) erlotinib, bortezomib, fulvestrant, sutent, letrozole, imatinib mesylate, PTK787/ZK 222584, oxaliplatin, 5-fluorouracil, leucovorin, rapamycin, lapatinib, lonafarnib, sorafenib, gefitinib, AG1478, AG1571, thiotepa, cyclophosphamide, busulfan, improsulfan, piposulfan, benzodopa, carboquone, meturedopa, uredopa, ethylenimine, altretamine, triethylenemelamine, triethylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin 1, cryptophycin 8, dolastatin, duocarmycin, KW-2189, CB1-TM1, eleutherobin, pancratistatin, sarcodictyin, spongistatin, chlorambucil, chlornaphazine, cholophosphamide, estramustine, ifosfamide, mechlorethamine, melphalan, novembichin, phenesterine, prednimustine, trofosfamide, uracil mustard, carmustine, chlorozotocin, fotemustine, lomustine, nimustine, ranimnustine, calicheamicin, calicheamicin gamma 1, calicheamicin omega 1, dynemicin, dynemicin A, clodronate, esperamicin, neocarzinostatin chromophore, aclacinomysins, actinomycin, antrmycin, azaserine, bleomycins, cactinomycin, carabicin, carninomycin, carzinophilin, chromomycins, dactinomycin, daunorubicin, detorubucin, 6-diazo-5-oxo-L-norleucine, doxorubicin, morpholino-doxorubicin, cyanomorpholino-doxorubicin, 2-pyrrolino-doxorubicin, liposomal doxorubicin, deoxydoxorubicin, epirubicin, esorubicin, marcellomycin, mitomycin C, mycophenolic acid, nogalamycin, olivomycins, peplomycin, potfiromycin, puromycin, quelamycin, rodorubicin, streptomigrin, streptozocin, tubercidin, ubenimex, zinostatin, zorubicin, 5-fluorouracil, denopterin, methotrexate, pteropterin, trimetrexate, fludarabine, 6-mercaptopurine, thiamiprine, thiguanine, ancitabine, azacitidine, 6-azauridine, carmofur, cytarabine, dideoxyuridine, doxifluridine, enocitabine, floxuridine, calusterone, dromostanolone, propionate, epitiostanol, mepitiostane, testolactone, aminoglutethimide, mitotane, trilostane, folinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, 2-ethylhydrazide, procarbazine, polysaccharide-k, razoxane, rhizoxin, sizofiran, spirogermanium, tenuazonic acid, triaziquone, 2,2,2-trichlorotriethylamine, T-2 toxin, verracurin A, roridin A, anguidine, urethane, vindesine, dacarbazine, mannomustine, mitobronitol, mitolactol, pipobroman, gacytosine, arabinoside, cyclophosphamide, thiotepa, paclitaxel, paclitaxel, albumin-engineered nanoparticle formulation of paclitaxel, docetaxel, chlorambucil, gemcitabine, 6-thioguanine, mercaptopurine, cisplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, CPT-11, topoisomerase inhibitor RFS 2000, difluoromethylornithine, retinoic acid, capecitabine, or a pharmaceutically acceptable salt, solvate or acid thereof;
(b) monokines, lymphokines, traditional polypeptide hormones, parathyroid hormones, thyroxine, relaxin, prorelaxin, glycoprotein hormone, follicle stimulating hormone, thyroid stimulating hormone, luteinizing hormone, hepatic growth factor fibroblast growth factor, prolactin, placental lactogen, tumor necrosis factor, tumor necrosis factor-α, tumor necrosis factor-β, mullerian inhibiting substance, mouse gonadotropin-associated peptide, inhibin, activin, vascular endothelial growth factor, thrombopoietin, erythropoietin, osteoinductive factor, interferon, interferon-α, interferon-β, interferon-γ, colony stimulating factor (CSF), macrophage-CSF, granulocyte-macrophage-CSF, granulocyte-CSF, interleukin (IL), IL-1, IL-1a, IL-2, IL-3, IL-4, IL-5, IL-6, IL-7, IL-8, IL-9, IL-10, IL-11, IL-12, tumor necrosis factor, polypeptide factor, LIF, kit ligand, or a combination thereof;
(c) diphtheria toxin, botulinum toxin, tetanus toxin, decentretoxin, cholera toxin, amanitin, α-amanitin, pyrrolobenzodiazepines, pyrrolobenzodiazepine derivatives, indolinobenzodiazepines, pyridinobenzodiazepines, tetrodotoxin, brevetoxin, ciguatoxin, ricin, AM toxin, auristatin, tubulysin, geldanamycin, maytansinoid, calicheamycin, daunomycin, doxorubicin, methotrexate, vindesine, SG2285, dolastatin, dolastatin analog, auristatin, cryptophycin, camptothecin, rhizoxin, rhizoxin derivatives, CC-1065, CC-1065, analogs or derivatives, duocarmycin, enediyne antibiotics, esperamicin, epothilone, toxoid, or a combination thereof;
(d) an affinity ligand, wherein the affinity ligand is a substrate, an inhibitor, an active agent, a neurotransmitter, a radioisotope, or a combination thereof;
(e) a radioactive label, 32P, 35S, a fluorescent dye, an electron dense reagent, an enzyme, biotin, streptavidin, dioxigenin, hapten, an immunogenic protein, a nucleic acid molecule with a sequence complementary to a target, or a combination thereof;
(f) an immunomodulatory compound, an anticancer agent, an anti-viral agent, an anti-bacterial agent, an anti-fungal agent, an anti-parasitic agent, or a combination thereof;
(g) tamoxifen, raloxifene, droloxifene, 4-hydroxytamoxifen, trioxifene, keoxifene, LY117018, onapristone, or toremifene;
(h) 4 (5)-imidazole, aminoglutethimide, megestrol acetate, exemestane, letrozole, or anastrozole;
(i) flutamide, nilutamide, bicalutamide, leuprolide, goserelin, or troxacitabine;
(j) aromatase inhibitors;
(k) protein kinase inhibitors;
(l) lipid kinase inhibitors;
(m) antisense oligonucleotides;
(n) ribozymes;
(o) vaccines; and
(p) anti-angiogenic agents.

In certain embodiments, the active agent is

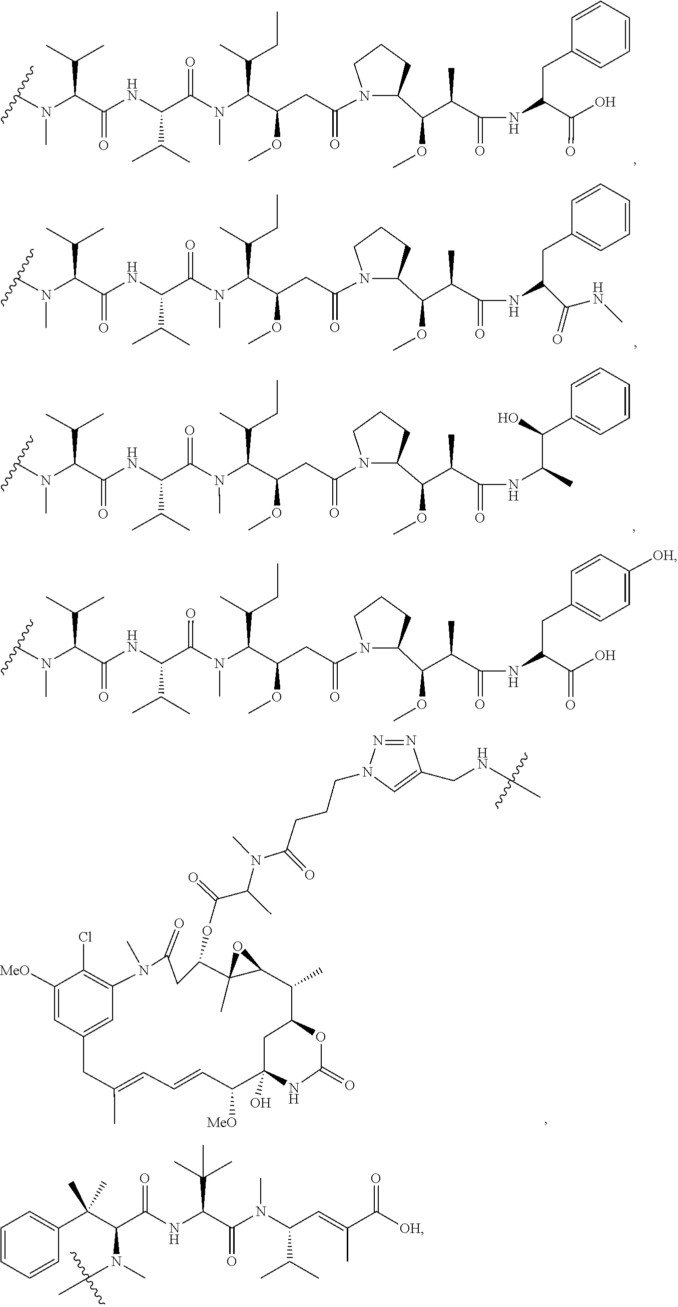

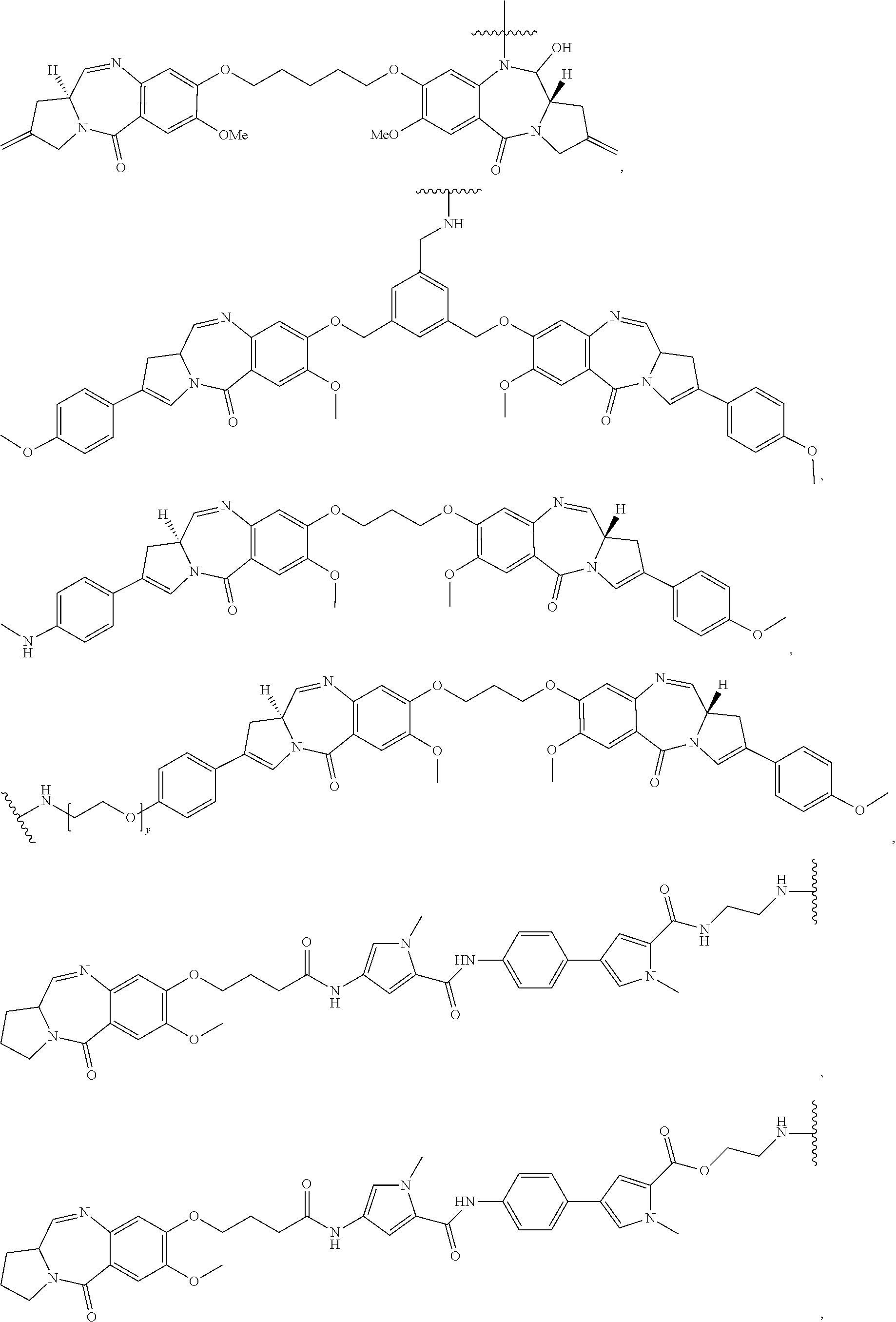

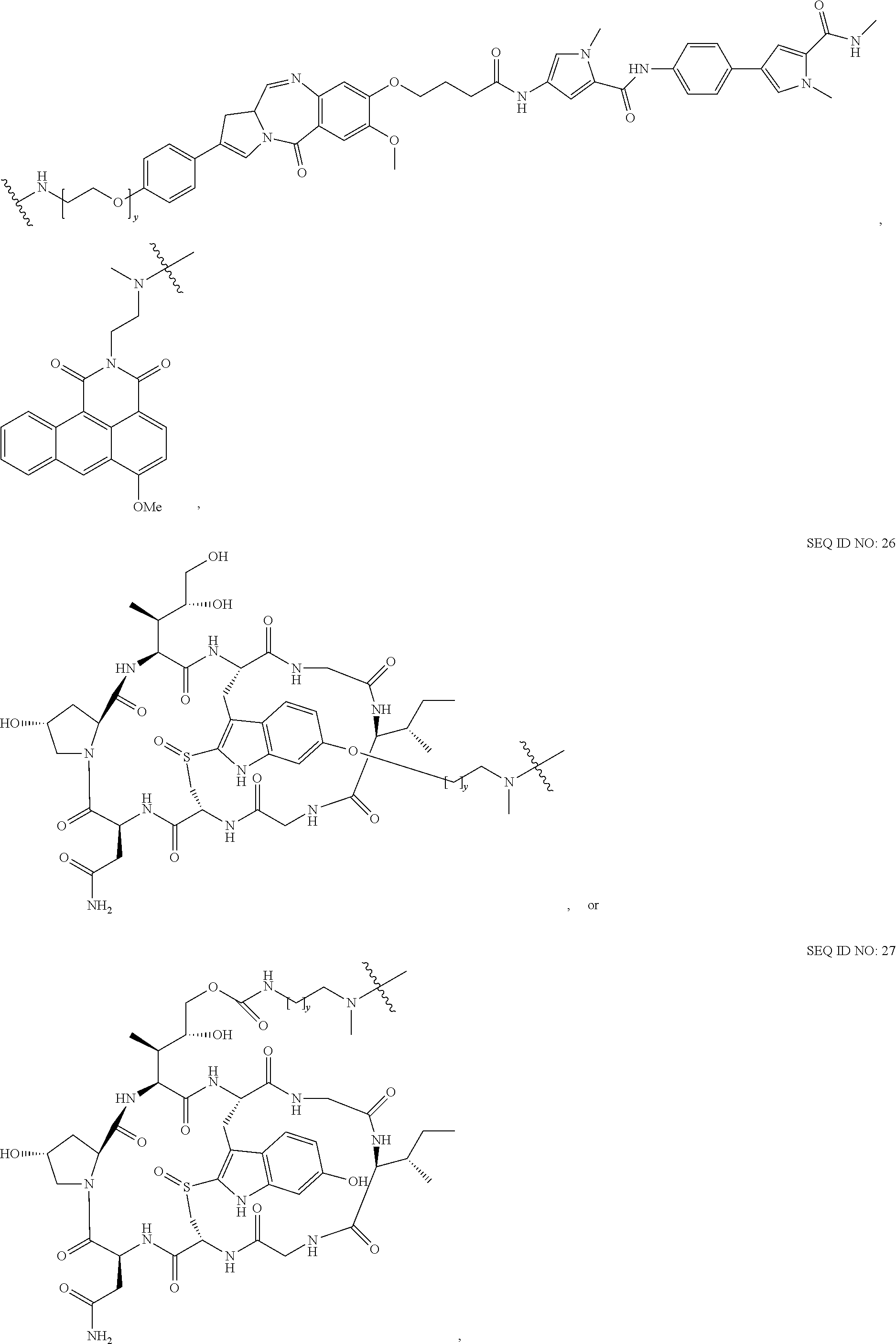

wherein y is 1 to 10; and
the wavy line indicates a connection to the conjugate.

In certain embodiments, the active agent is a pyrrolobenzodiazepine dimer, position N10 of the pyrrolobenzodiazepine dimer is substituted with X or position N′10 is substituted with X′, wherein X or X′ links the pyrrolobenzodiazepine dimer to the linker;
X and X [symbol] re each independently —C(O)O—* or —C(O)—*; and
* refers to a binding site between the pyrrolobenzodiazepine dimer and the linker.

In certain preferred embodiments, the pyrrolobenzodiazepine dimer is represented by Formula III:

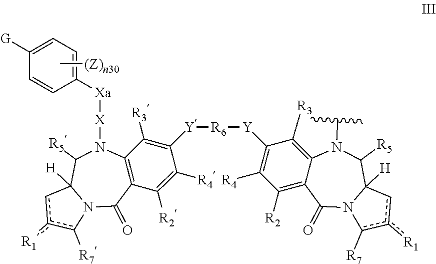

wherein
the wavy line indicates a connection point to the conjugate (e.g., the carbonyl of formula IIa connected to B′);

a dotted line represents an optional double bond;

R₁ and R₁ [symbol] re each independently selected from H, OH, ═O, —CH₂, CN, R^{m}, OR^{m}, —CH-R^{m} [symbol] ═C(R^{m} [symbol] )₂, O—SO₂—R^{m}, CO₂R^{m}, COR^{m}, halo, and dihalo;
R^{m} [symbol] s selected from R^{m}, CO₂R^{m}, COR^{m}, CHO, CO₂H, and halo;
R^{m} is selected from substituted or unsubstituted C₁₋₁₂ alkyl, substituted or unsubstituted C₂₋₁₂ alkenyl, substituted or unsubstituted C₂₋₁₂ alkynyl, substituted or unsubstituted C₅₋₂₀ aryl, substituted or unsubstituted C₃₋₆ heteroaryl, substituted or unsubstituted C₃₋₆ cycloalkyl, substituted or unsubstituted 3- to 7-membered heterocyclyl, substituted or unsubstituted 3- to 7-membered heterocycloalkyl, and substituted or unsubstituted 5- to 7-membered heteroaryl, wherein when the C₁₋₁₂ alkyl, C₂₋₁₂ alkenyl, C₂₋₁₂ alkynyl, C₅₋₂₀ aryl, C₅₋₂₀ heteroaryl, C₃₋₆ cycloalkyl, 3- to 7-membered heterocyclyl, 3- to 7-membered heterocycloalkyl, or 5- to 7-membered heteroaryl is substituted, the respective hydrogen atoms in the C₁₋₁₂ alkyl, C₂₋₁₂ alkenyl, C₂₋₁₂ alkynyl, C₅₋₂₀ aryl, C₅₋₂₀ heteroaryl, C₃₋₆ cycloalkyl, 3- to 7-membered heterocyclyl, 3- to 7-membered heterocycloalkyl, or 5- to 7-membered heteroaryl may each be independently replaced with methoxy, C₁₋₁₂ alkyl, C₂₋₁₂ alkenyl, C₂₋₁₂ alkynyl, C₅₋₂₀ aryl, C₅₋₂₀ heteroaryl, C₃₋₆ cycloalkyl, 3- to 7-membered heterocyclyl, 3- to 7-membered heterocycloalkyl, and 5- to 7-membered heteroaryl;
R₂, R₃, R₅, [symbol] and [symbol] are each independently selected from H, R^{m}, OH, OR^{m}, SH, SR^{m}, NH₂, NHR^{m}, NR^{m}R^{m} [symbol] NO₂, Me₃Sn, and halo;
R₄ and [symbol] are each independently selected from H, R^{m}, OH, OR^{m}, SH, SR^{m}, NH₂, NHR^{m}, NR^{m} [symbol] , NO₂, Me₃Sn, halo, substituted or unsubstituted C₁₋₆ alkyl, substituted or unsubstituted C₁₋₆ alkoxy, substituted or unsubstituted C₂₋₆ alkenyl, substituted or unsubstituted C₂₋₆ alkynyl, substituted or unsubstituted C₃₋₆ cycloalkyl, substituted or unsubstituted 3- to 7-membered heterocycloalkyl, substituted or unsubstituted C₅₋₁₂ aryl, substituted or unsubstituted 5- to 7-membered heteroaryl, —CN, —NCO, —ORⁿ, —OC(O)Rⁿ, —OC(O)NRⁿRⁿ [symbol] , —OS(O)Rⁿ, —OS(O)₂Rⁿ, —SRⁿ, —S(O)Rⁿ, —S(O)₂Rⁿ, —S(O)NRⁿRⁿ [symbol] , —S(O)₂NRⁿ [symbol] , —OS(O)NRⁿRⁿ [symbol] , —OS(O)₂NRⁿRⁿ [symbol] , —NRⁿRⁿ [symbol] , —NRⁿC(O)R^{o}, —NRⁿC(O)OR^{o}, —NRⁿC(O)NR^{o} [symbol] , —NRⁿS(O)R^{o}, —NRⁿS(O)₂R^{o}, —NRⁿS(O)NR^{o}R^{o} [symbol] , —NRⁿS(O)₂NR^{o}R^{o} [symbol] , —C(O)Rⁿ, —C(O)ORⁿ, and —C(O)NRⁿRⁿ [symbol] , wherein the hydrogen atoms in the C₁₋₆ alkyl, C₁₋₆ alkoxy, C₂₋₆ alkenyl, C₂₋₆ alkynyl, C₃₋₆ cycloalkyl, 3- to 7-membered heterocycloalkyl, C₅₋₁₂ aryl, and 5- to 7-membered heteroaryl may each be independently replaced with C₁₋₆ alkyl, C₁₋₆ alkoxy, C₂₋₆ alkenyl, C₂₋₆ alkynyl, C₃₋₆ cycloalkyl, 3- to 7-membered heterocycloalkyl, C₅₋₁₂ aryl, 5- to 7-membered heteroaryl, —OR^{p}, —OC(O)R^{p}, —OC(O)NR^{p}R^{p}, —OS(O)R^{p}, —OS(O)₂R^{p}, —SR^{p}, —S(O)R^{p}, —S(O)₂R^{p}, —S(O)NR^{p}R^{p}, —S(O)₂NR^{p}R^{p}, —OS(O)NR^{p}R^{p}, —OS(O)₂NR^{p}R^{p}, —NR^{p}R^{p}, —NR^{p}C(O)R^{q}, —NR^{p}C(O)OR^{q}, —NR^{p}C(O)NR^{q}H, —NR^{p}S(O)R⁴, —NR^{p}S(O)₂R⁴, —NR^{p}S(O)NR^{q}H, —NR^{p}S(O)₂NR⁴H, —C(O)R^{p}, —C(O)OR^{p}, or —C(O)NR^{p}R^{p} when the C₁₋₆ alkyl, C₁₋₆ alkoxy, C₂₋₆ alkenyl, C₂₋₆ alkynyl, C₃₋₆ cycloalkyl, 3- to 7-membered heterocycloalkyl, C₅₋₁₂ aryl, and 5- to 7-membered heteroaryl;
Rⁿ, Rⁿ [symbol] , R^{o}, R^{o}R^{p}, R^{p′}, and R^{q} are each independently selected from H, C₁₋₇ alkyl, C₂₋₇ alkenyl, C₂₋₇ alkynyl, C₃₋₁₃ cycloalkyl, 3- to 7-membered heterocycloalkyl, C₆₋₁₀ aryl, and 5- to 7-membered heteroaryl;
X is selected from —C(O)O—, —S(O)O—, —C(O)—, —C(O)NR—, —S(O)₂NR—, —P(O)R [symbol] R—, —S(O)NR—, and —PO₂NR—;
Xa is a bond or substituted or unsubstituted C₁₋₆ alkylene, wherein C₁₋₆ alkylene is substituted with C₁₋₈ alkyl, or C₃₋₈ cycloalkyl when substituted;
R and R [symbol] ach independently denote H, OH, NH₂, ONH₂, NHNH₂, substituted or unsubstituted C₁₋₈ alkyl, substituted or unsubstituted C₃₋₈ cycloalkyl, substituted or unsubstituted C₁₋₈ alkoxy, substituted or unsubstituted C₁₋₈ alkylthio, substituted or unsubstituted C₃₋₂₀ heteroaryl, substituted or unsubstituted C₅₋₂₀ aryl, or mono- or di-C₁₋₈ alkylamino, wherein the C₁₋₈ alkyl, C₃₋₈ cycloalkyl, C₁₋₈ alkoxy, C₁₋₈ alkylthio, C₃₋₂₀ heteroaryl, and C₅₋₂₀ aryl are substituted with a substituent selected from OH, N3, CN, NO₂, SH, NH₂, ONH₂, NHNH₂, halo, C₁₋₆ alkyl, C₁₋₆ alkoxy, and C₆₋₁₂ aryl when substituted;
Y and Y [symbol] re each independently selected from O, S, and N(H);
R⁶ is a substituted or unsubstituted saturated or unsaturated C₃₋₁₂ hydrocarbon chain, wherein the chain may be interrupted by one or more heteroatoms, NMe, or a substituted or unsubstituted aromatic ring, the chain or aromatic ring may be substituted with —NH, —NR¹, —NHC(O)R^{m}, —NHC(O)CH₂—[OCH₂CH₂]ₙ—R, or —[CH₂CH₂O]ₙ—R at any one or more positions of hydrogen atoms on the chain or aromatic ring or unsubstituted, wherein R^{m} and R are each as defined for R^{m} and R above, and n is 1 to 12; and
R⁷ and R^{7′} are each independently H, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ cycloalkyl, substituted or unsubstituted 3- to 7-membered heterocycloalkyl, substituted or unsubstituted C₆₋₁₀ aryl, substituted or unsubstituted 5- to 7-membered heteroaryl, —OR^{r}, —OC(O)R^{r}, —OC(O)NR^{r}R^{r} [symbol] , —OS(O)R^{r}, —OS(O)₂R^{r}, —SR^{r}, —S(O)R^{r}, —S(O)₂R^{r}, —S(O)NR^{r}R^{r} [symbol] , —S(O)₂NR^{r}R^{r} [symbol] , —OS(O)NR^{r}R^{r}, —OS(O)₂NR^{r}R^{r} [symbol] , —NR^{r}R^{r} [symbol] , —NR^{r}C(O)R^{s}, —NR^{r}C(O)OR^{s}, —NR^{r}C(O)NR^{s}R^{s} [symbol] , —NR^{r}S(O)R^{s}, —NR^{r}S(O)₂R^{s}, —NR^{r}S(O)NR^{s}R^{s} [symbol] , —NR^{r}S(O)₂NR^{s}R^{s}, —C(O)R^{r}, —C(O)OR^{s}, or —C(O)NR^{r}R^{r} [symbol] , wherein the hydrogen atoms in the C₁₋₆ alkyl, C₂₋₆ alkenyl, C₂₋₆ alkynyl, C₃₋₆ cycloalkyl, 3- to 7-membered heterocycloalkyl, C₆₋₁₀ aryl, and 5- to 7-membered heteroaryl may each be independently replaced with C₁₋₆ alkyl, C₂₋₆ alkenyl, C₂₋₆ alkynyl, C₃₋₆ cycloalkyl, 3- to 7-membered heterocycloalkyl, C₆₋₁₀ aryl, 5- to 7-membered heteroaryl, —OR^{t}, —OC(O)R^{t}, —OC(O)NR^{t}R^{t} [symbol] , —OS(O)R^{t}, —OS(O)₂R^{t}, —SR^{t}, —S(O)R^{t}, —S(O)₂R^{t}, —S(O)NR^{t}R^{t} [symbol] , —S(O)₂NR^{t}R^{t} [symbol] , —OS(O)NR^{t}R^{t} [symbol] , —OS(O)₂NR^{t}R^{t} [symbol] , —NR^{t}R^{t} [symbol] , —NR^{t}C(O)R^{u}, —NR^{t}C(O)OR^{u}, —NR^{t}C(O)NR^{u}R^{u} [symbol] , —NR^{t}S(O)R^{u}, —NR^{t}S(O)₂R^{u}, —NR^{t}S(O)NR^{u}R^{u} [symbol] , —NR^{t}S(O)₂NR^{u}R^{u} [symbol] , —C(O)R^{t}, —C(O)OR^{t}, or —C(O)NR^{t}R^{t} [symbol] when the C₁₋₆ alkyl, C₂₋₆ alkenyl, C₂₋₆ alkynyl, C₃₋₆ cycloalkyl, 3- to 7-membered heterocycloalkyl, C₆₋₁₀ aryl, and 5- to 7-membered heteroaryl;
R^{r}, R^{r} [symbol] , R^{s}, R^{s} [symbol] , R^{t}, R^{t} [symbol] , R^{u}, and R^{u} [symbol] re each independently selected from H, C₁₋₇ alkyl, C₂₋₇ alkenyl, C₂₋₇ alkynyl, C₃₋₁₃ cycloalkyl, 3- to 7-membered heterocycloalkyl, C₅₋₁₀ aryl, and 5- to 7-membered heteroaryl;
G is a glucuronide group or a galactoside group;
each Z is selected from H, C₁₋₈ alkyl, halo, NO₂, CN,

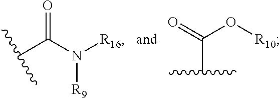

R₉, R₁₀, and R₁₆ are each independently selected from H, C₁₋₈ alkyl, C₂₋₆ alkenyl, C₁₋₆ alkoxy, alkyloxyalkyl, and methyloxyethyl; and
n30 is 1 to 3.

In certain embodiments, a dotted line represents presence of a double bond between the carbons bearing R₁ and R₇ or R₁ [symbol] and R₇ [symbol] .

In certain embodiments, R₁ is R^{m} and R^{m} is selected from substituted or unsubstituted C₁₋₆ alkyl, substituted or unsubstituted C₂₋₆ alkenyl, substituted or unsubstituted C₅₋₇ aryl, and substituted or unsubstituted C₃₋₆ heteroaryl.

In certain embodiments, R₂, R₃, and R₅ are each independently H or OH.

In certain embodiments, R₄ is C₁₋₆ alkoxy.

In certain embodiments, R₄ is methoxy, ethoxy, or butoxy.

In certain embodiments, X is selected from —C(O)O—, —C(O)—, and —C(O)NR—; and
Rs each independently denote H, OH, N3, CN, NO₂, SH, NH₂, ONH₂, NHNH₂, halo, substituted or unsubstituted C₁₋₈ alkyl, or substituted or unsubstituted C₁₋₈ alkoxy, wherein C₁₋₈ alkyl or C₁₋₈ alkoxy is substituted with OH, N₃, CN, NO₂, SH, NH₂, ONH₂, NNH₂, or halo when substituted.

In certain embodiments, X is —C(O)NR—.

In certain embodiments, R⁶ is a substituted or unsubstituted saturated or unsaturated C₃₋₈ hydrocarbon chain, wherein
one or more of the carbon atoms of the hydrocarbon chain is replaced by a heteroatom or a substituted or unsubstituted aromatic ring, wherein the heteroatom is O, S, or N(H) and the aromatic ring is benzene, pyridine, imidazole, or pyrazole, and the chain or aromatic ring may be substituted with —NHC(O)CH₂—[OCH₂CH₂]ₙ—R or —[CH₂CH₂O]ₙ— R at any one or more positions of hydrogen atoms on the chain or aromatic ring; and
n is 1 to 6.

In certain embodiments, n is 1 to 10.

In certain embodiments, Xa is a bond or C₁₋₃ alkylene.

In certain embodiments, Z is H,

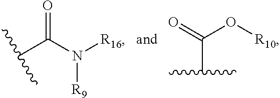

wherein R₉, R₁₀, and R₁₆ are each independently selected from H, C₁₋₃ alkyl, C₁₋₃ alkoxy, and allyloxymethyl.

In certain embodiments, R₉ is methyloxyalkyl.

In certain embodiments, R₁₀ is methyloxyalkyl.

In certain embodiments, R₁₆ is methyloxyalkyl.

In certain embodiments, R₉, R₁₀, or R₁₆ is —(CH₂CH₂O)ₘ—(CH₂)ₘ₂CH₃, further wherein m is 1-6 and m2 is 0-2.3

In certain embodiments, R₂ is H.

In certain embodiments, R₃ is H.

In certain embodiments, R₇ is H.

In certain embodiments, R₄ is alkoxy (e.g., methoxy).

In certain embodiments, R₅ is OH.

In certain embodiments, R₁ is ═CH₂, CH₃, or phenyl, optionally substituted with methoxy.

In certain embodiments, Y is O.

In certain embodiments, R₂′ is H.

In certain embodiments, R₃′ is H.

In certain embodiments, R₇′ is H.

In certain embodiments, R₄′ is alkoxy (e.g., methoxy).

In certain embodiments, R₅′ is OH.

In certain embodiments, R₁′ is ═CH₂, CH₃, or phenyl, optionally substituted with methoxy.

In certain embodiments, Y′ is O.

In certain embodiments, X is —C(O)O—In certain embodiments, Xa is CH₂.

In certain embodiments, G is a glucuronide group.

In certain embodiments, G is

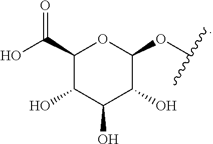 [SMILES: *O[C@@H]1O[C@H](C(=O)O)[C@@H](O)[C@H](O)[C@H]1O]

In certain embodiments, n30 is 1.

In certain embodiments, Z is

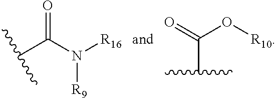

In certain embodiments, R₉ is H.

In certain embodiments, R₁₆ is alkyloxyalkyl (e.g., methoxyethyl).

In certain embodiments, Z is

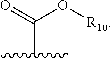

In certain embodiments, R₁₀ is alkyl (e.g., methyl).

In certain embodiments, R₆ is alkyl (e.g., pentyl).

In certain embodiments, the conjugate comprises a structure selected from:

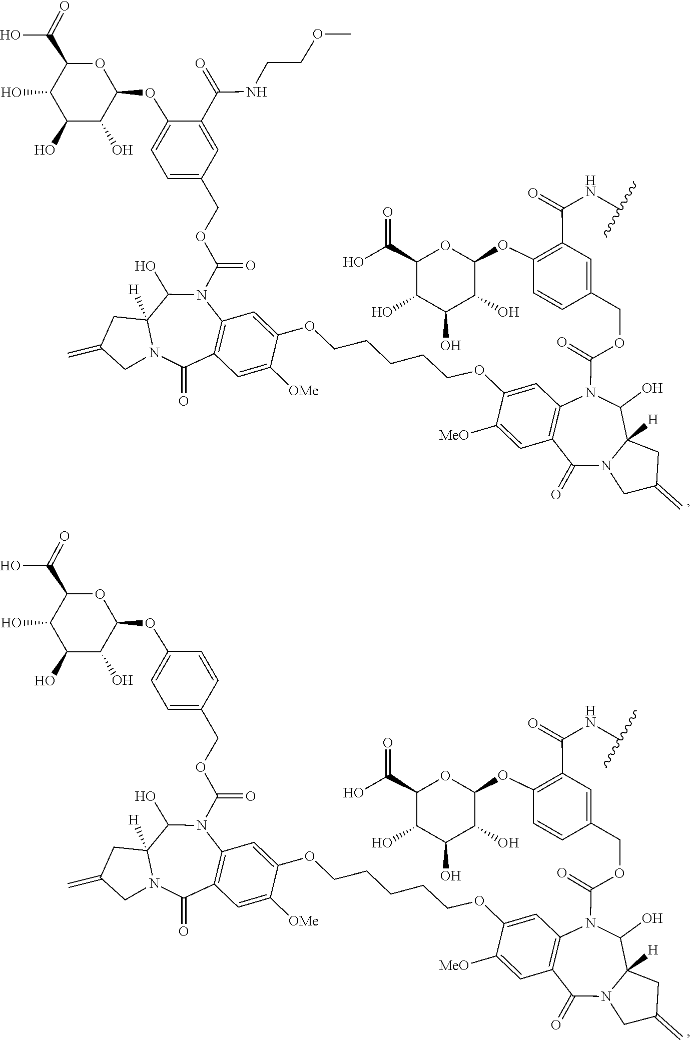 [SMILES: *NC(=O)c1cc(COC(=O)N2c3cc(OCCCCCOc4cc5c(cc4OC)C(=O)N4CC(=C)C[C@H]4C(O)N5C(=O)OCc4ccc(O[C@@H]5O[C@H](C(=O)O)[C@@H](O)[C@H](O)[C@H]5O)c(C(=O)NCCOC)c4)c(OC)cc3C(=O)N3CC(=C)C[C@H]3C2O)ccc1O[C@@H]1O[C@H](C(=O)O)[C@@H](O)[C@H](O)[C@H]1O ; *NC(=O)c1cc(COC(=O)N2c3cc(OCCCCCOc4cc5c(cc4OC)C(=O)N4CC(=C)C[C@H]4C(O)N5C(=O)OCc4ccc(O[C@@H]5O[C@H](C(=O)O)[C@@H](O)[C@H](O)[C@H]5O)cc4)c(OC)cc3C(=O)N3CC(=C)C[C@H]3C2O)ccc1O[C@@H]1O[C@H](C(=O)O)[C@@H](O)[C@H](O)[C@H]1O]

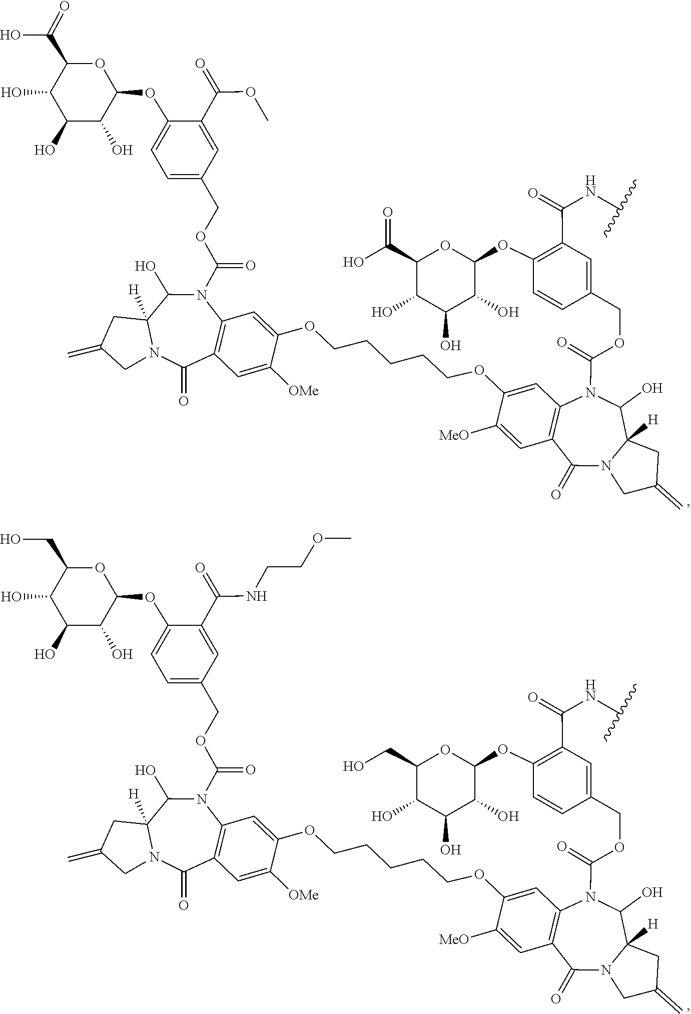 [SMILES: *NC(=O)c1cc(COC(=O)N2c3cc(OCCCCCOc4cc5c(cc4OC)C(=O)N4CC(=C)C[C@H]4C(O)N5C(=O)OCc4ccc(O[C@@H]5O[C@H](C(=O)O)[C@@H](O)[C@H](O)[C@H]5O)c(C(=O)OC)c4)c(OC)cc3C(=O)N3CC(=C)C[C@H]3C2O)ccc1O[C@@H]1O[C@H](C(=O)O)[C@@H](O)[C@H](O)[C@H]1O ; *NC(=O)c1cc(COC(=O)N2c3cc(OCCCCCOc4cc5c(cc4OC)C(=O)N4CC(=C)C[C@H]4C(O)N5C(=O)OCc4ccc(O[C@@H]5O[C@H](CO)[C@@H](O)[C@H](O)[C@H]5O)c(C(=O)NCCOC)c4)c(OC)cc3C(=O)N3CC(=C)C[C@H]3C2O)ccc1O[C@@H]1O[C@H](CO)[C@@H](O)[C@H](O)[C@H]1O]

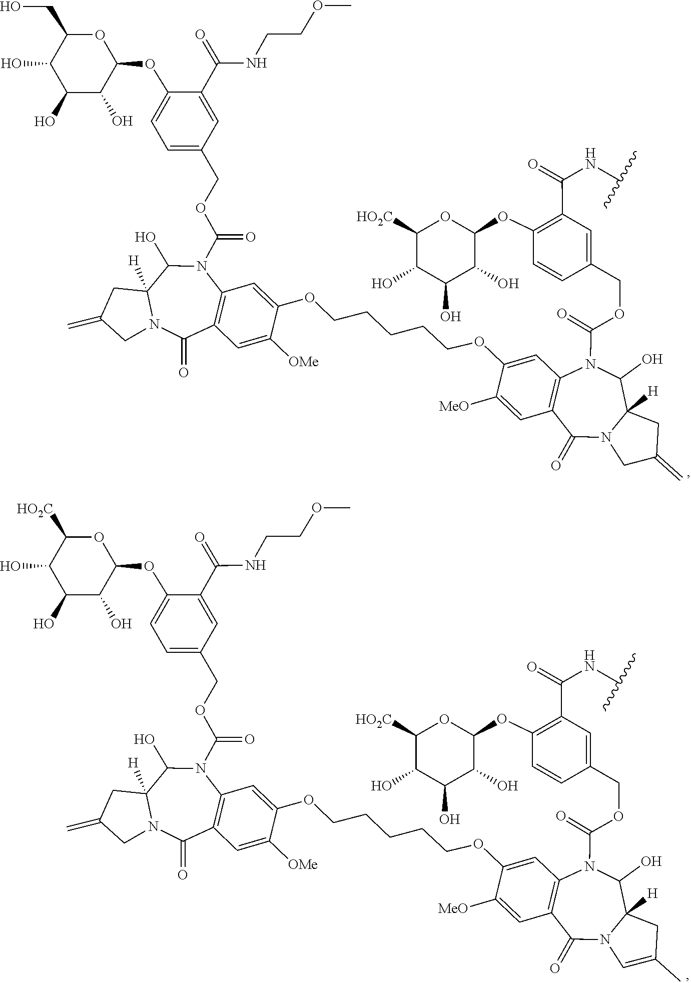 [SMILES: *NC(=O)c1cc(COC(=O)N2c3cc(OCCCCCOc4cc5c(cc4OC)C(=O)N4CC(=C)C[C@H]4C(O)N5C(=O)OCc4ccc(O[C@@H]5O[C@H](CO)[C@@H](O)[C@H](O)[C@H]5O)c(C(=O)NCCOC)c4)c(OC)cc3C(=O)N3CC(=C)C[C@H]3C2O)ccc1O[C@@H]1O[C@H](C(=O)O)[C@@H](O)[C@H](O)[C@H]1O ; *NC(=O)c1cc(COC(=O)N2c3cc(OCCCCCOc4cc5c(cc4OC)C(=O)N4CC(=C)C[C@H]4C(O)N5C(=O)OCc4ccc(O[C@@H]5O[C@H](C(=O)O)[C@@H](O)[C@H](O)[C@H]5O)c(C(=O)NCCOC)c4)c(OC)cc3C(=O)N3C=C(C)C[C@H]3C2O)ccc1O[C@@H]1O[C@H](C(=O)O)[C@@H](O)[C@H](O)[C@H]1O]

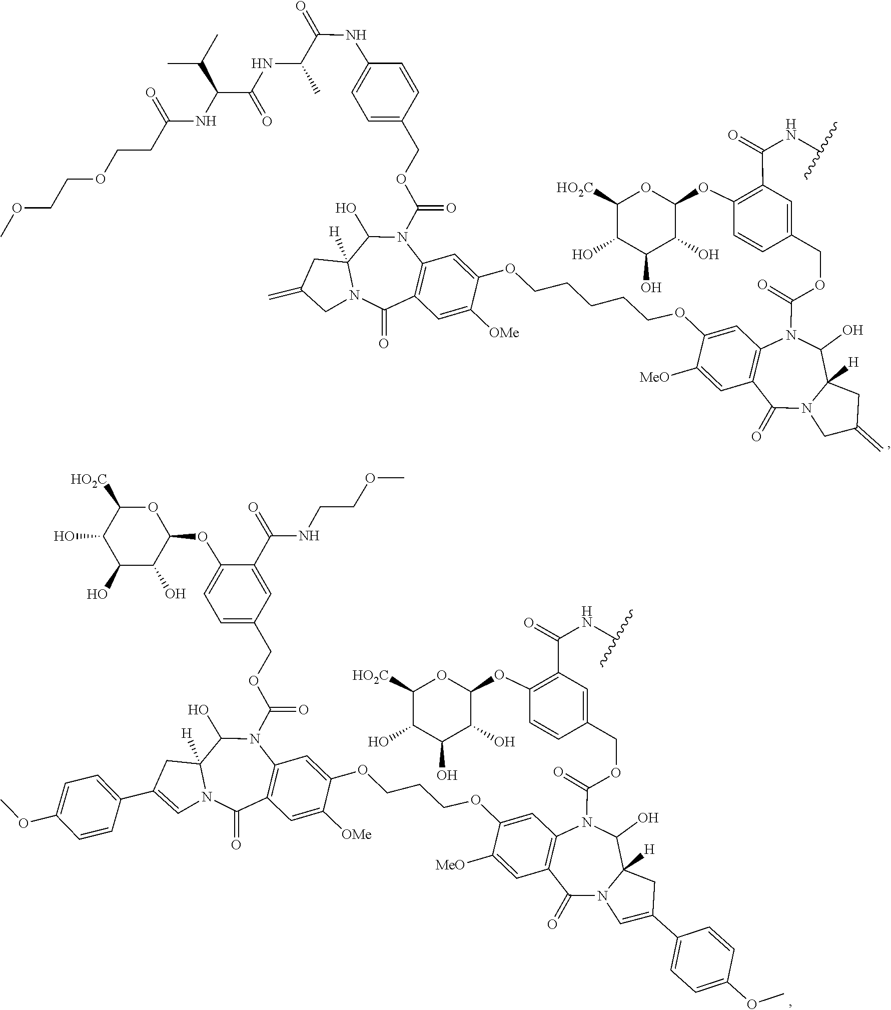

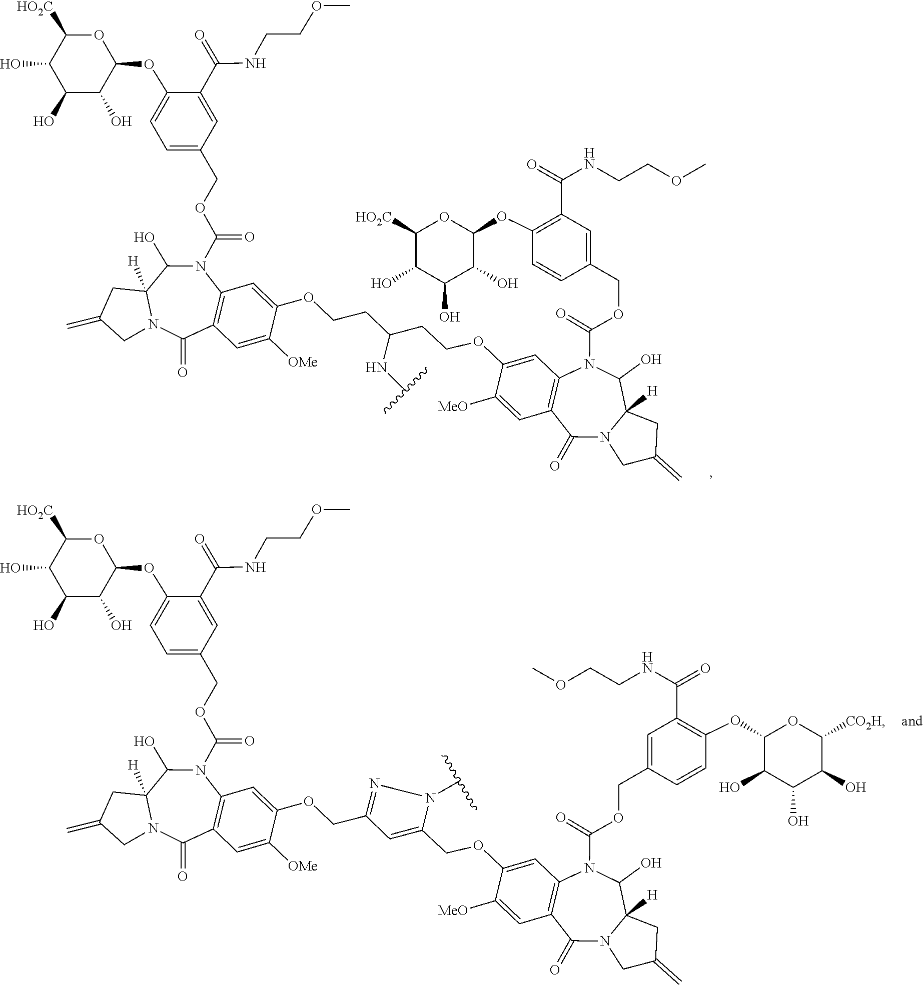 [SMILES: *NC(CCOc1cc2c(cc1OC)C(=O)N1CC(=C)C[C@H]1C(O)N2C(=O)OCc1ccc(O[C@@H]2O[C@H](C(=O)O)[C@@H](O)[C@H](O)[C@H]2O)c(C(=O)NCCOC)c1)CCOc1cc2c(cc1OC)C(=O)N1CC(=C)C[C@H]1C(O)N2C(=O)OCc1ccc(O[C@@H]2O[C@H](C(=O)O)[C@@H](O)[C@H](O)[C@H]2O)c(C(=O)NCCOC)c1 ; *n1nc(COc2cc3c(cc2OC)C(=O)N2CC(=C)C[C@H]2C(O)N3C(=O)OCc2ccc(O[C@@H]3O[C@H](C(=O)O)[C@@H](O)[C@H](O)[C@H]3O)c(C(=O)NCCOC)c2)cc1COc1cc2c(cc1OC)C(=O)N1CC(=C)C[C@H]1C(O)N2C(=O)OCc1ccc(O[C@@H]2O[C@H](C(=O)O)[C@@H](O)[C@H](O)[C@H]2O)c(C(=O)NCCOC)c1]

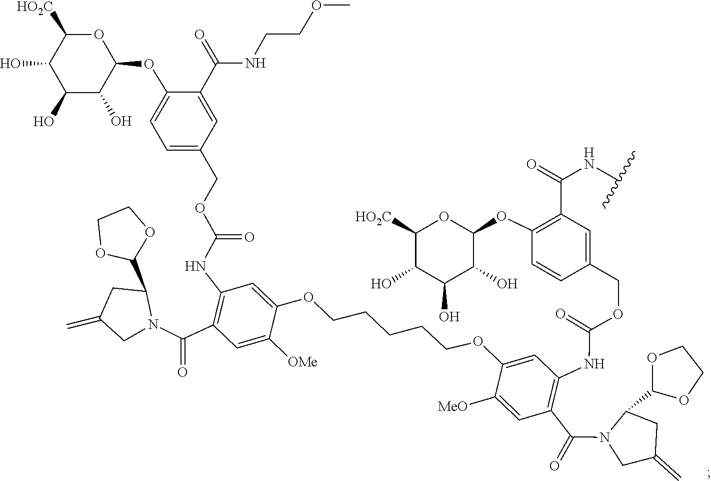 [SMILES: *NC(=O)c1cc(COC(=O)Nc2cc(OCCCCCOc3cc(NC(=O)OCc4ccc(O[C@@H]5O[C@H](C(=O)O)[C@@H](O)[C@H](O)[C@H]5O)c(C(=O)NCCOC)c4)c(C(=O)N4CC(=C)C[C@H]4C4OCCO4)cc3OC)c(OC)cc2C(=O)N2CC(=C)C[C@H]2C2OCCO2)ccc1O[C@@H]1O[C@H](C(=O)O)[C@@H](O)[C@H](O)[C@H]1O]

wherein the bond overlaid with a dashed line represents a connection point to L.

In certain embodiments, the conjugate comprises a structure selected from:

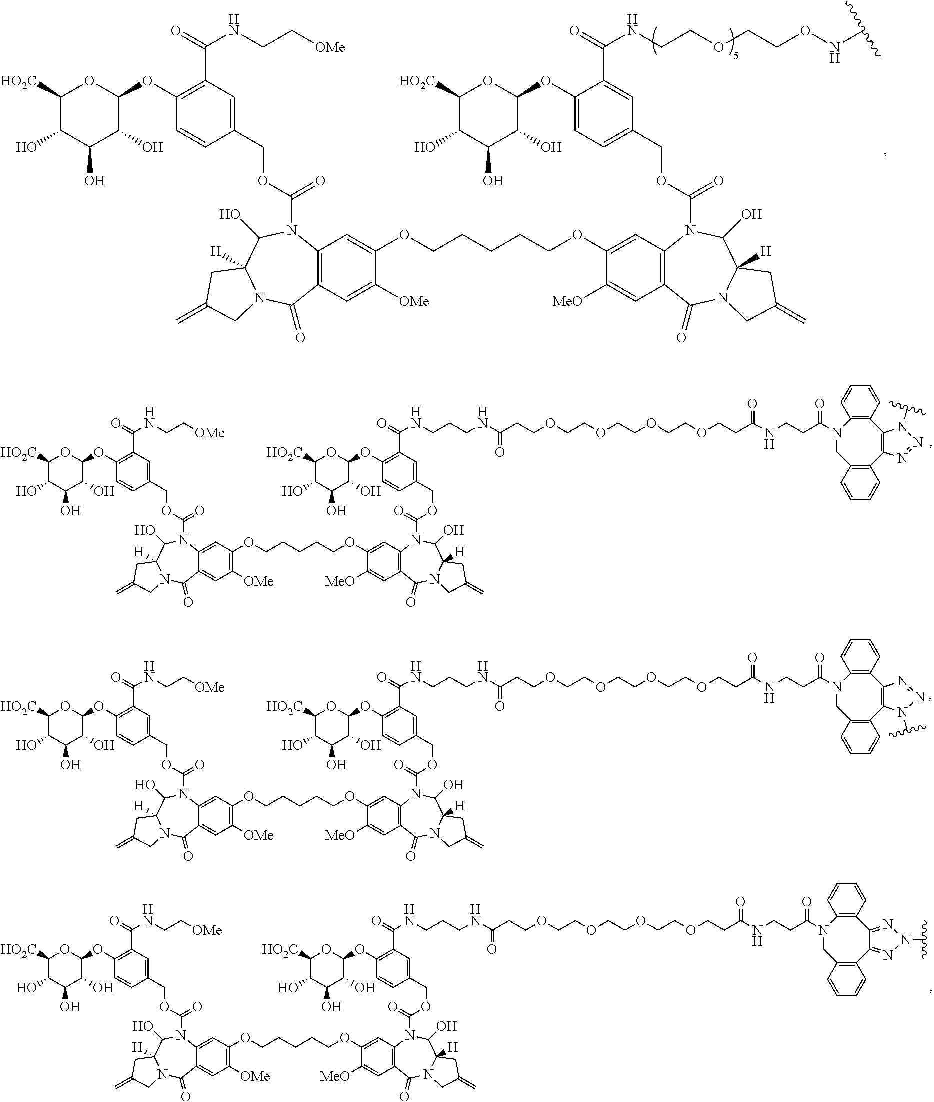

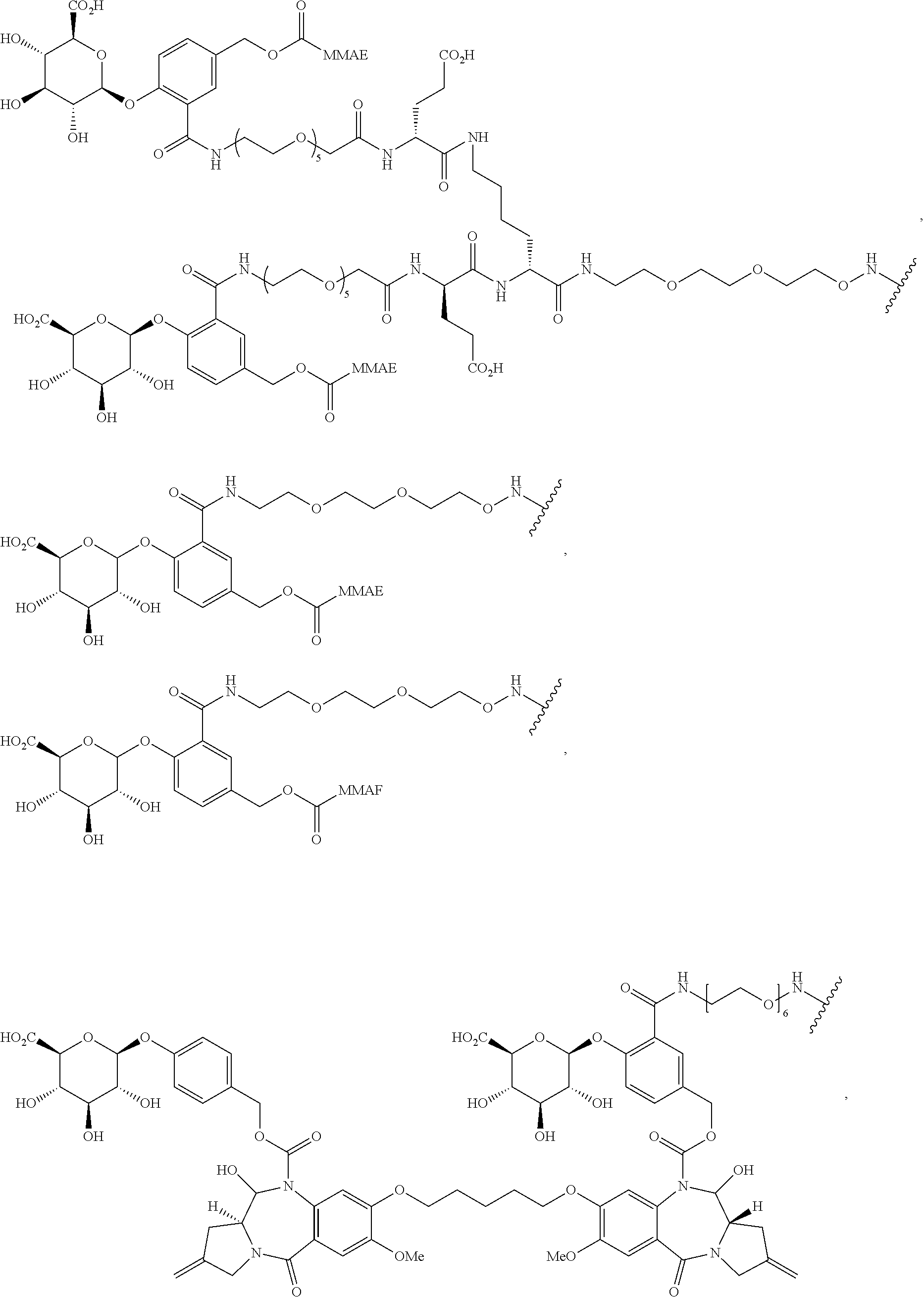

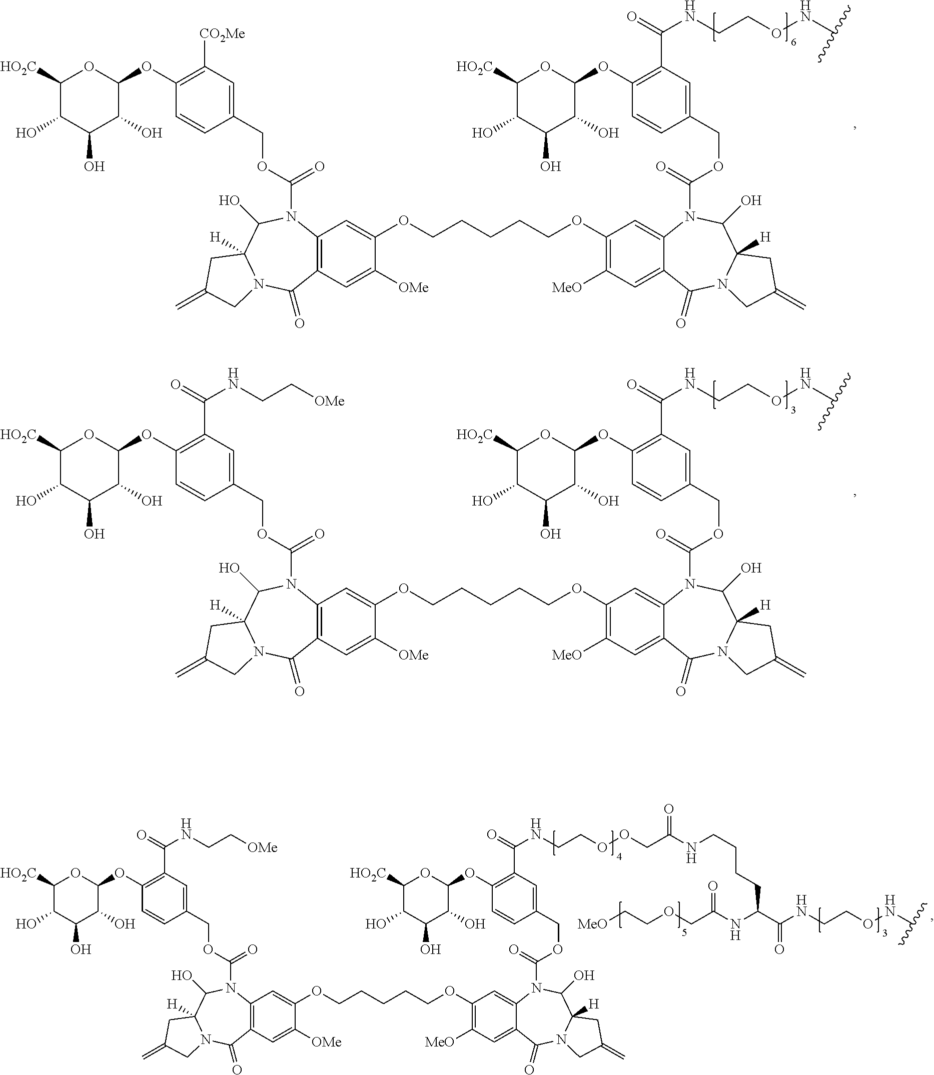

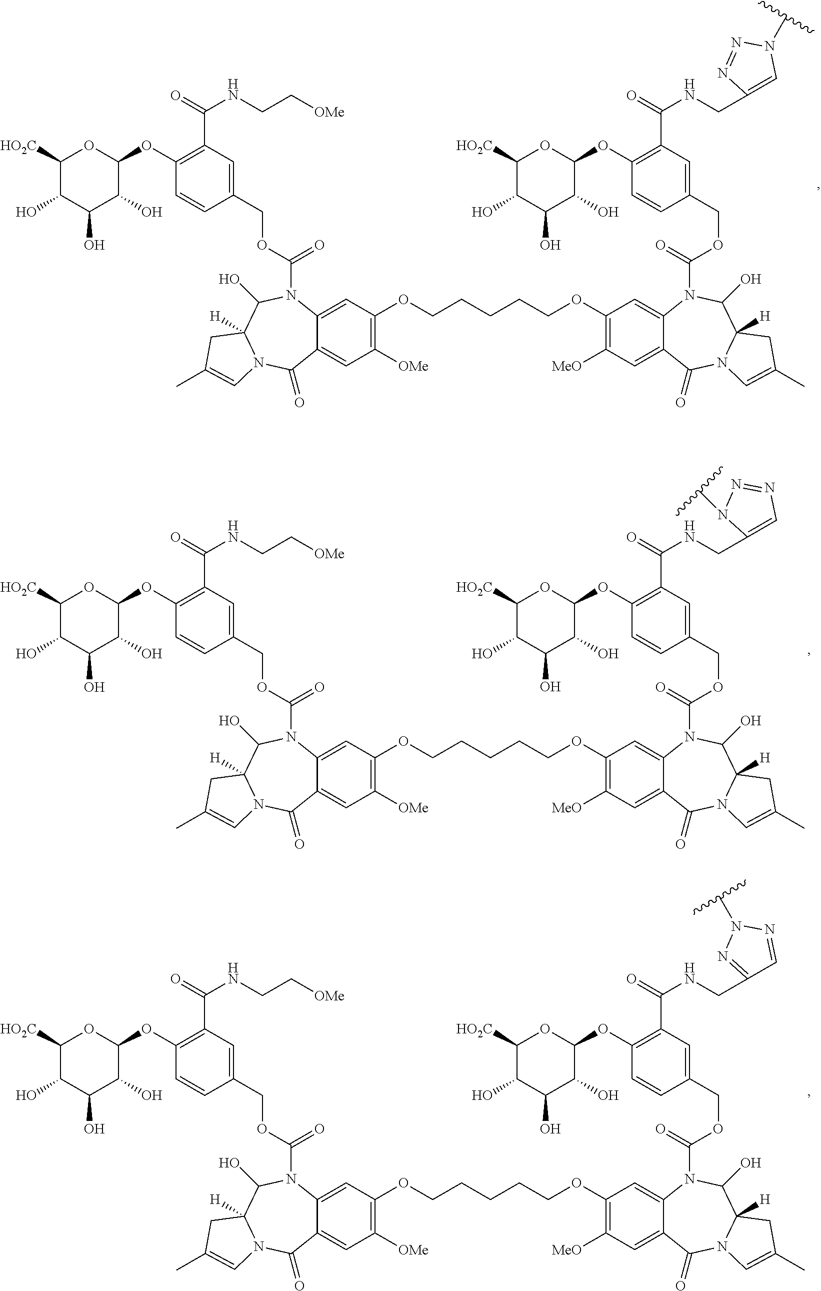

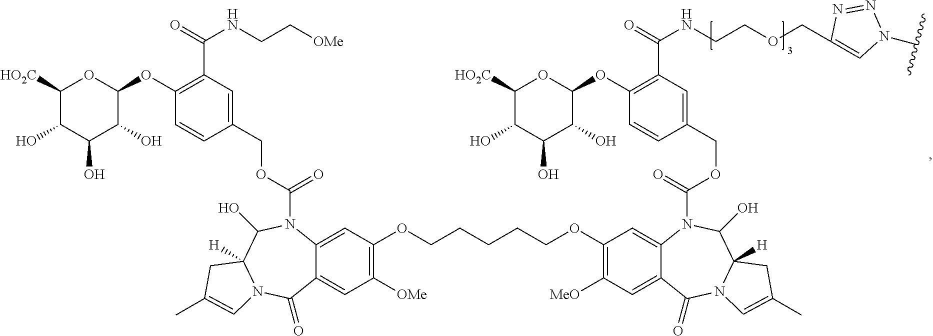

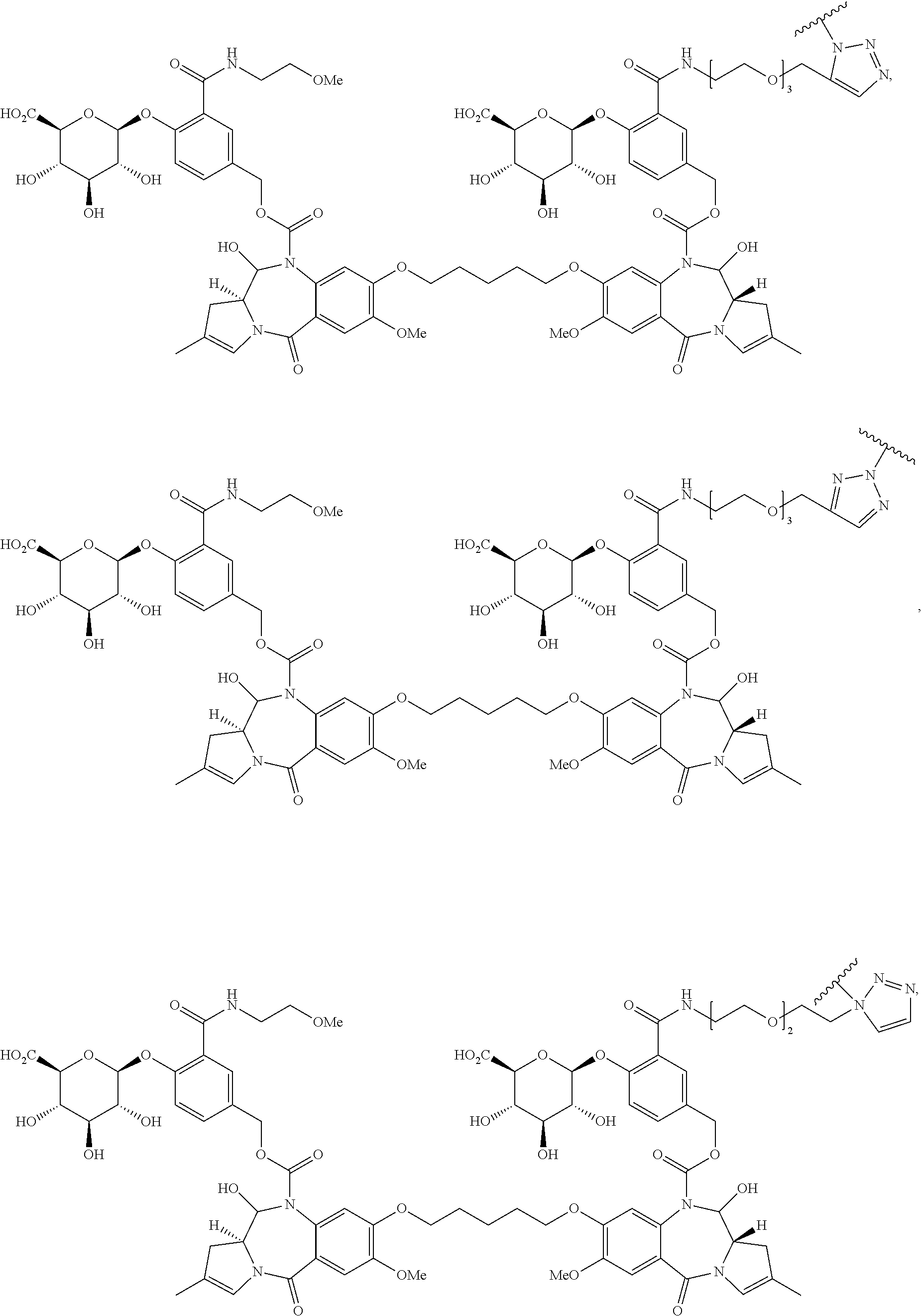

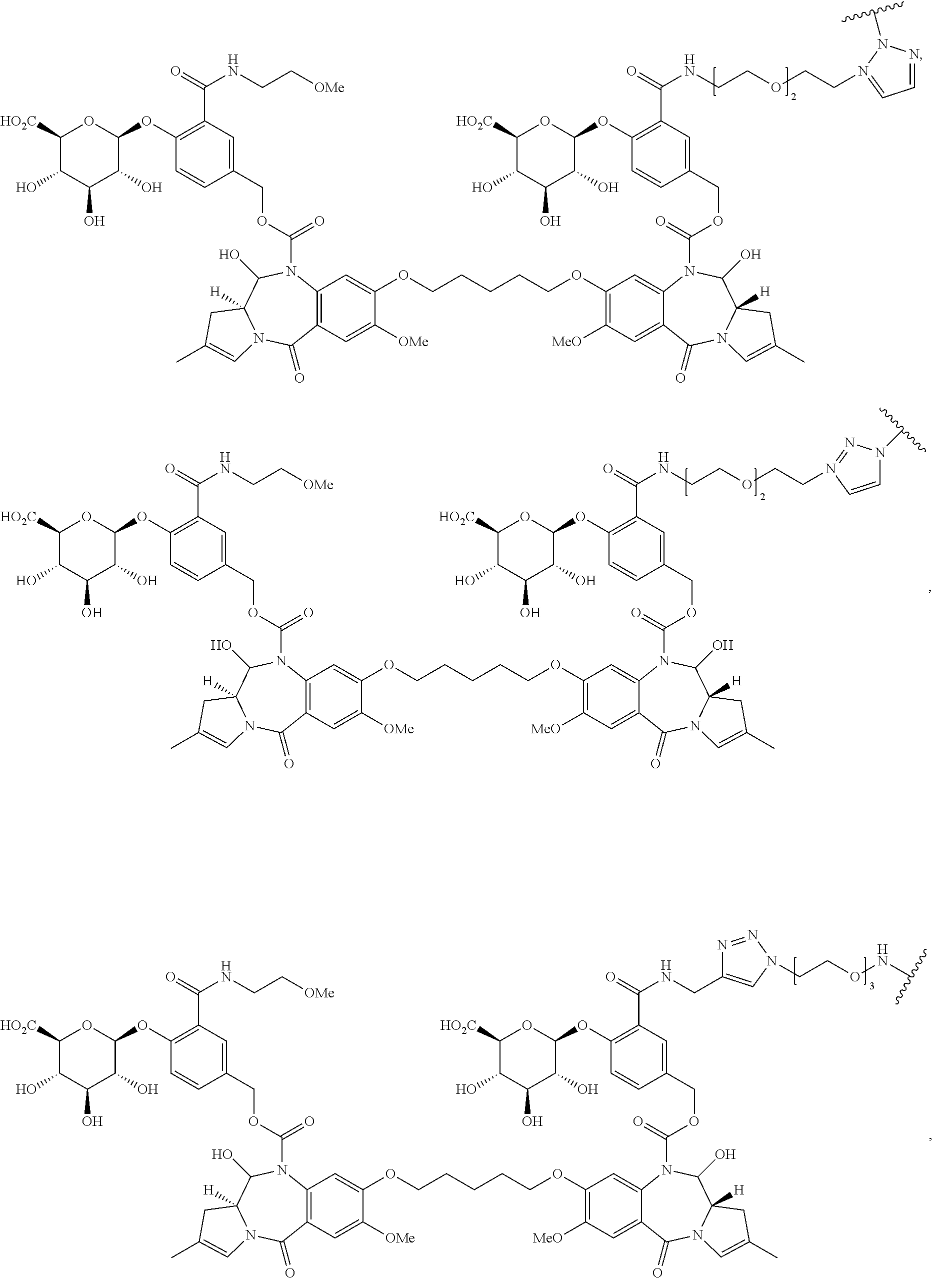

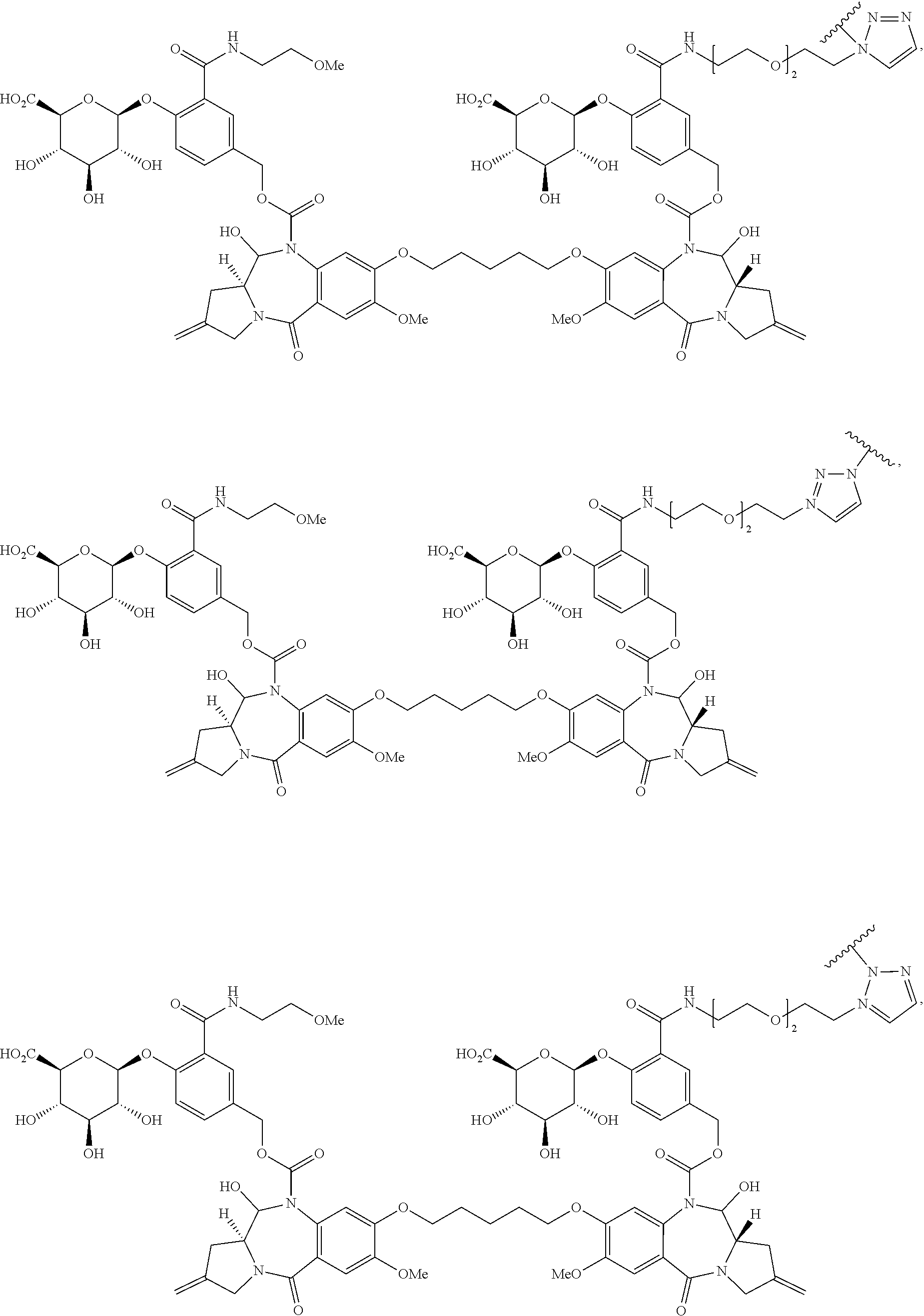

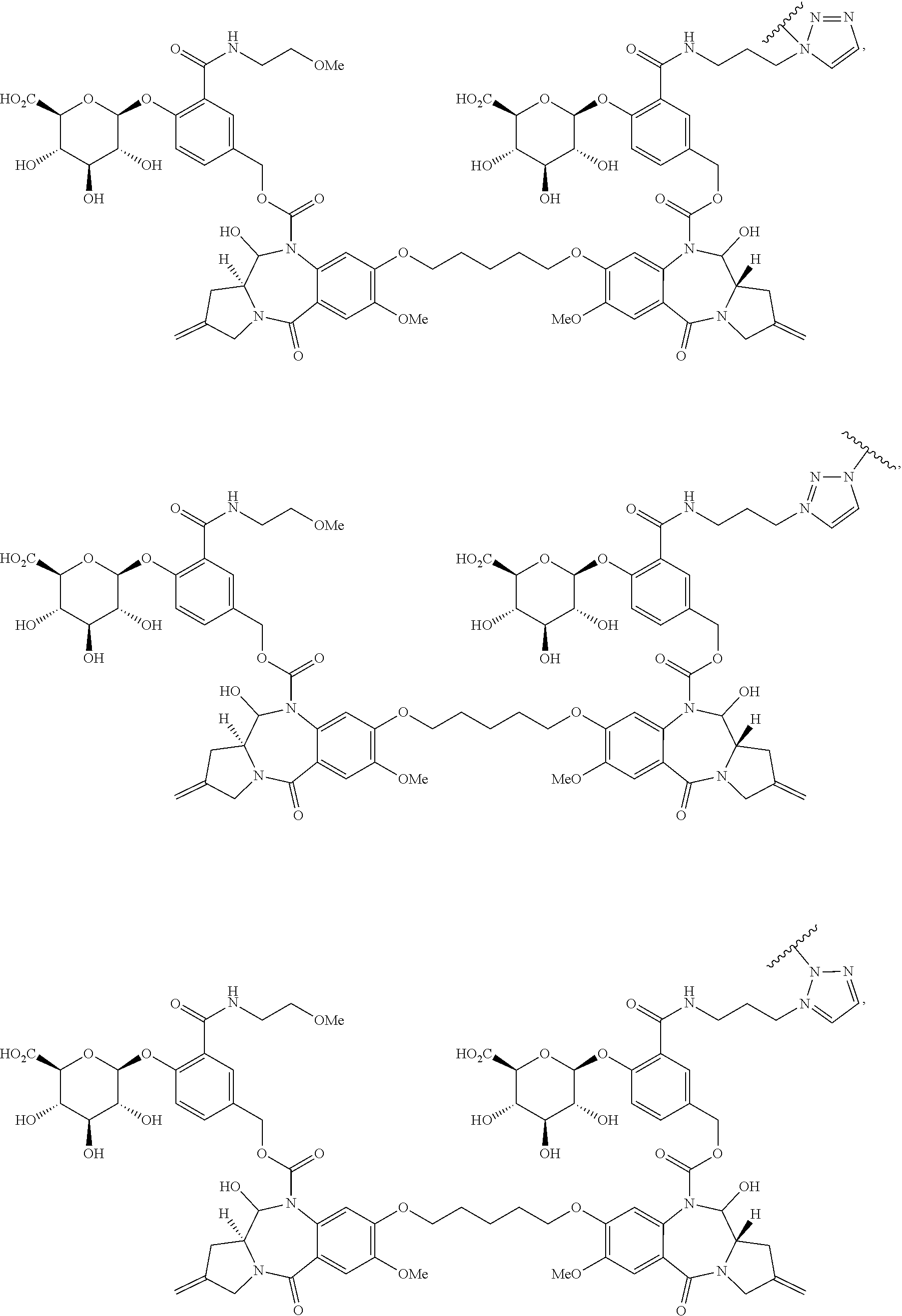

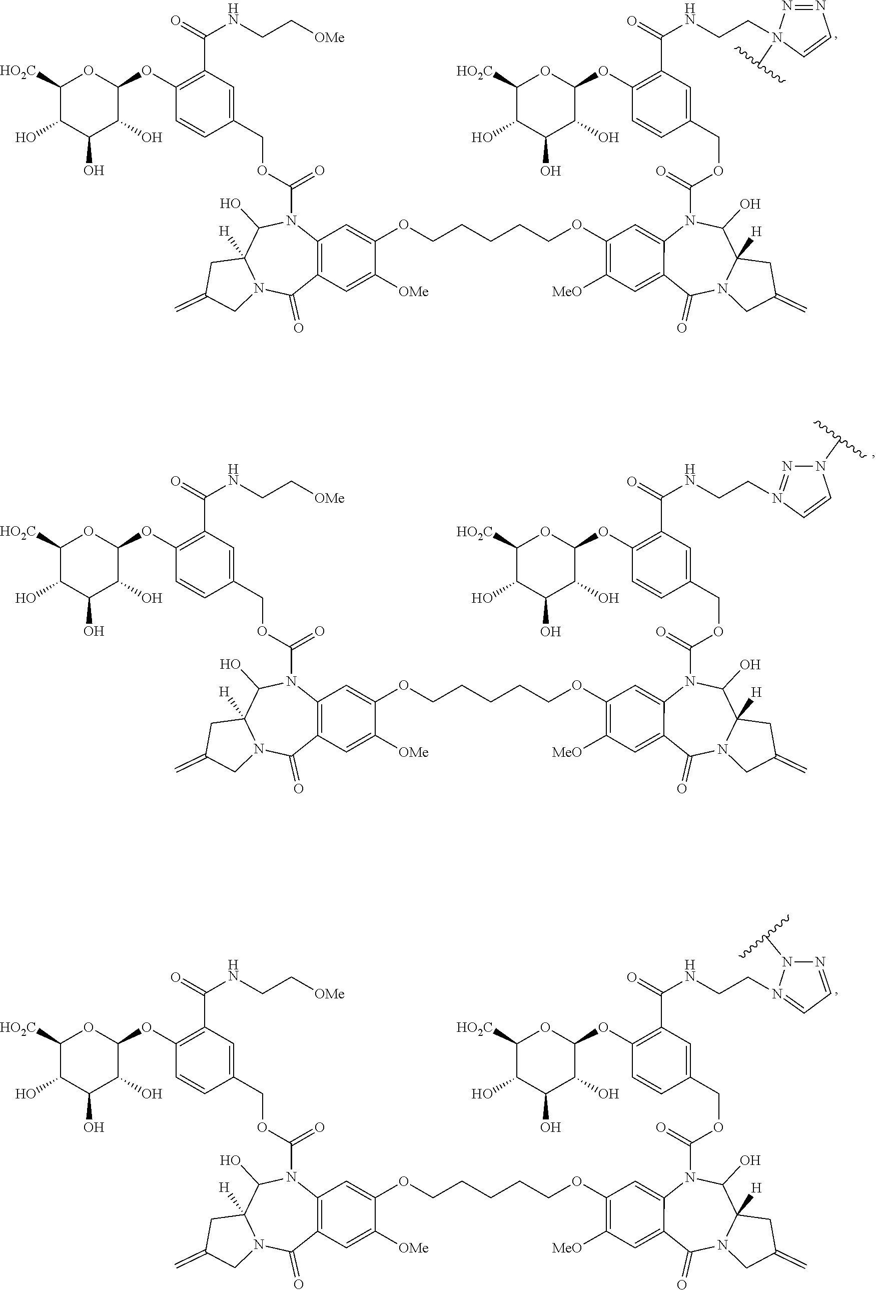

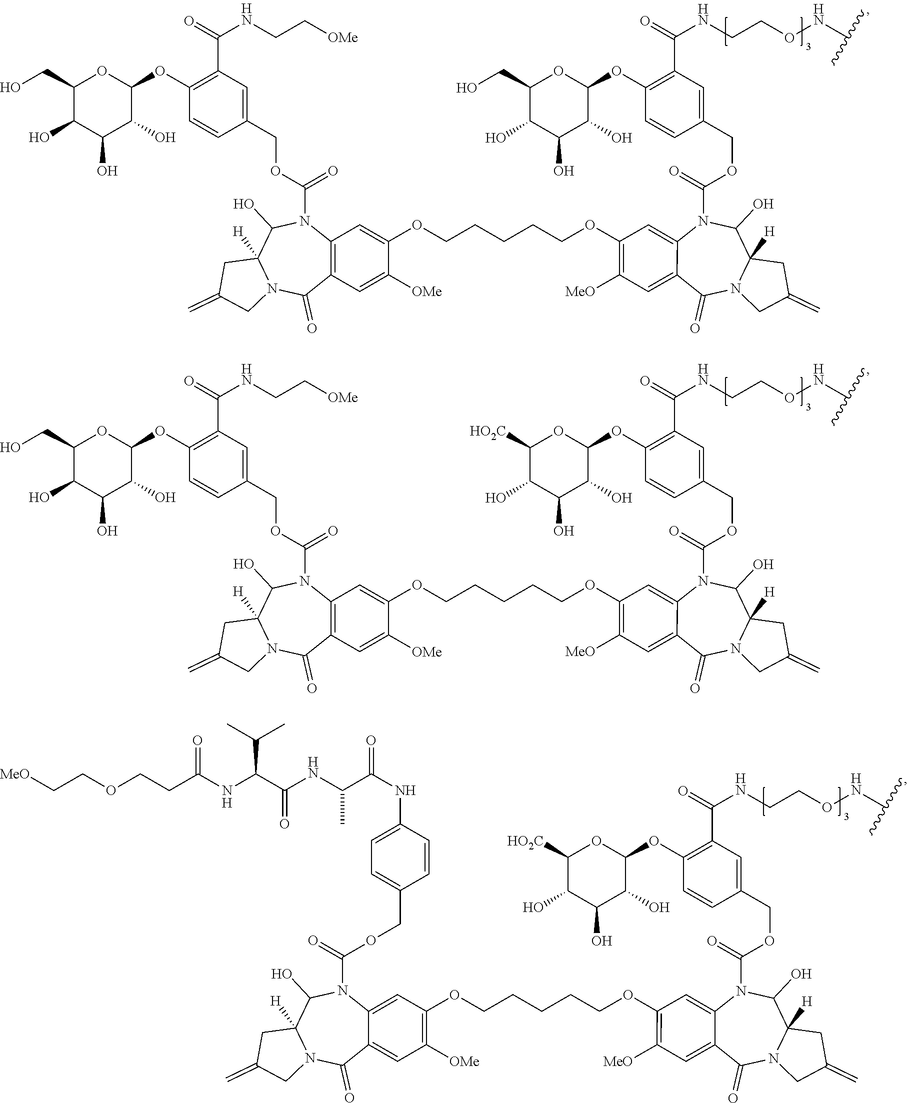

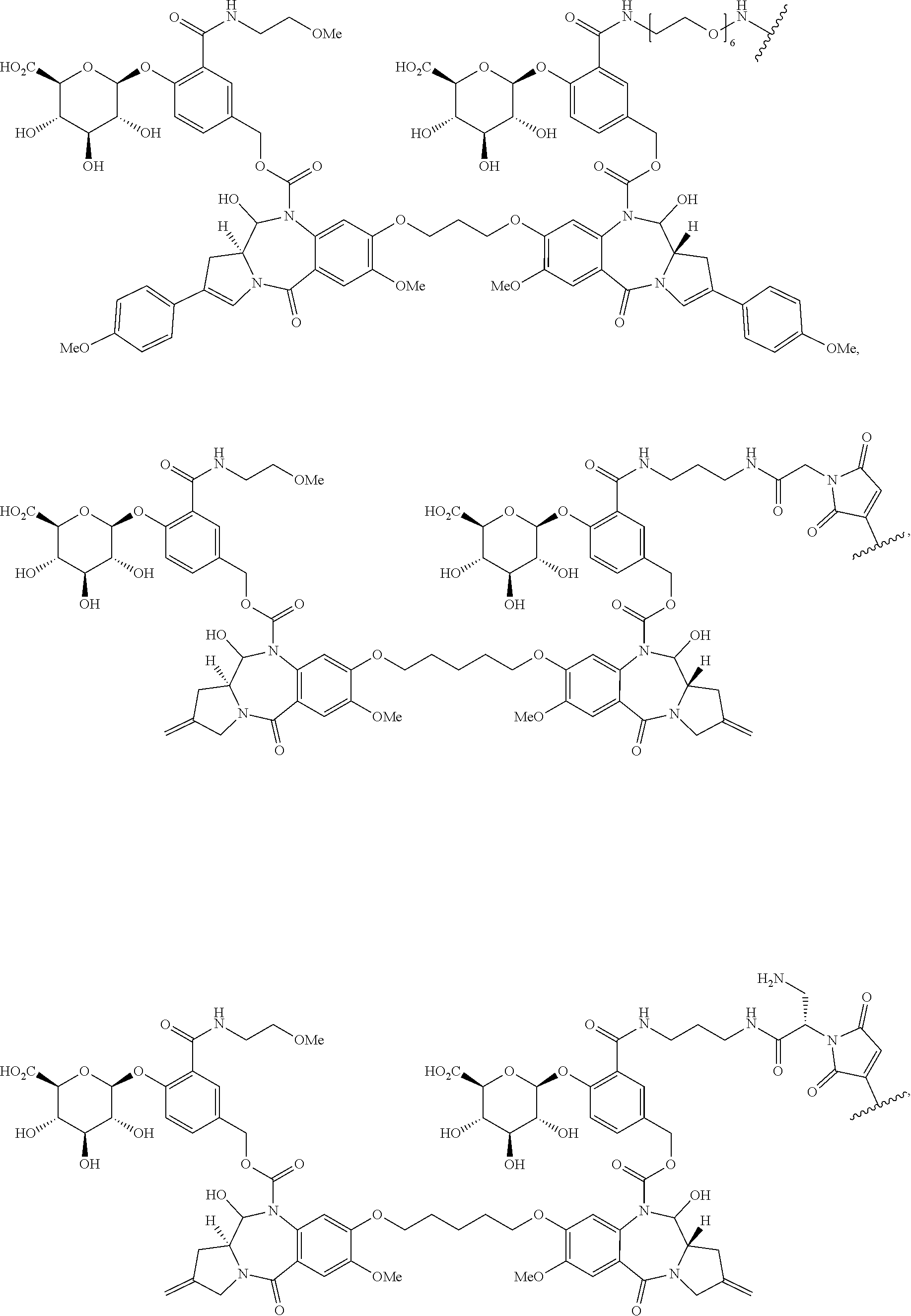

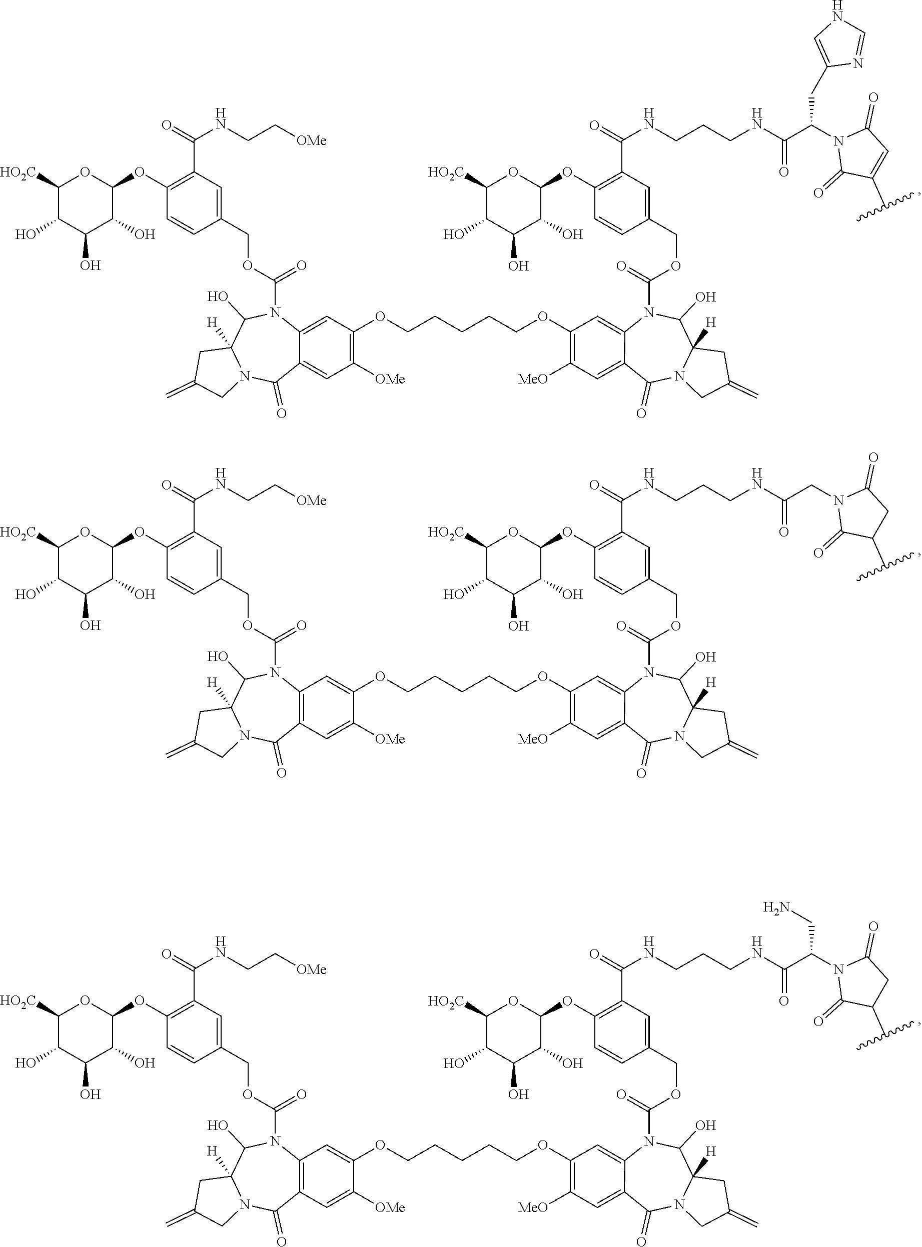

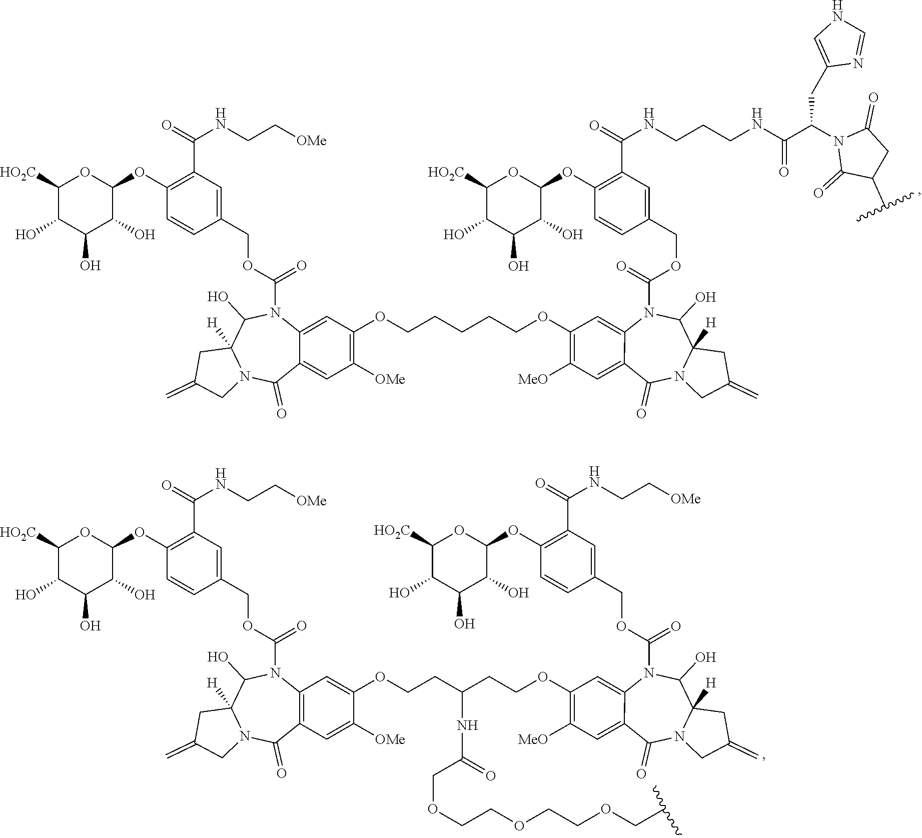

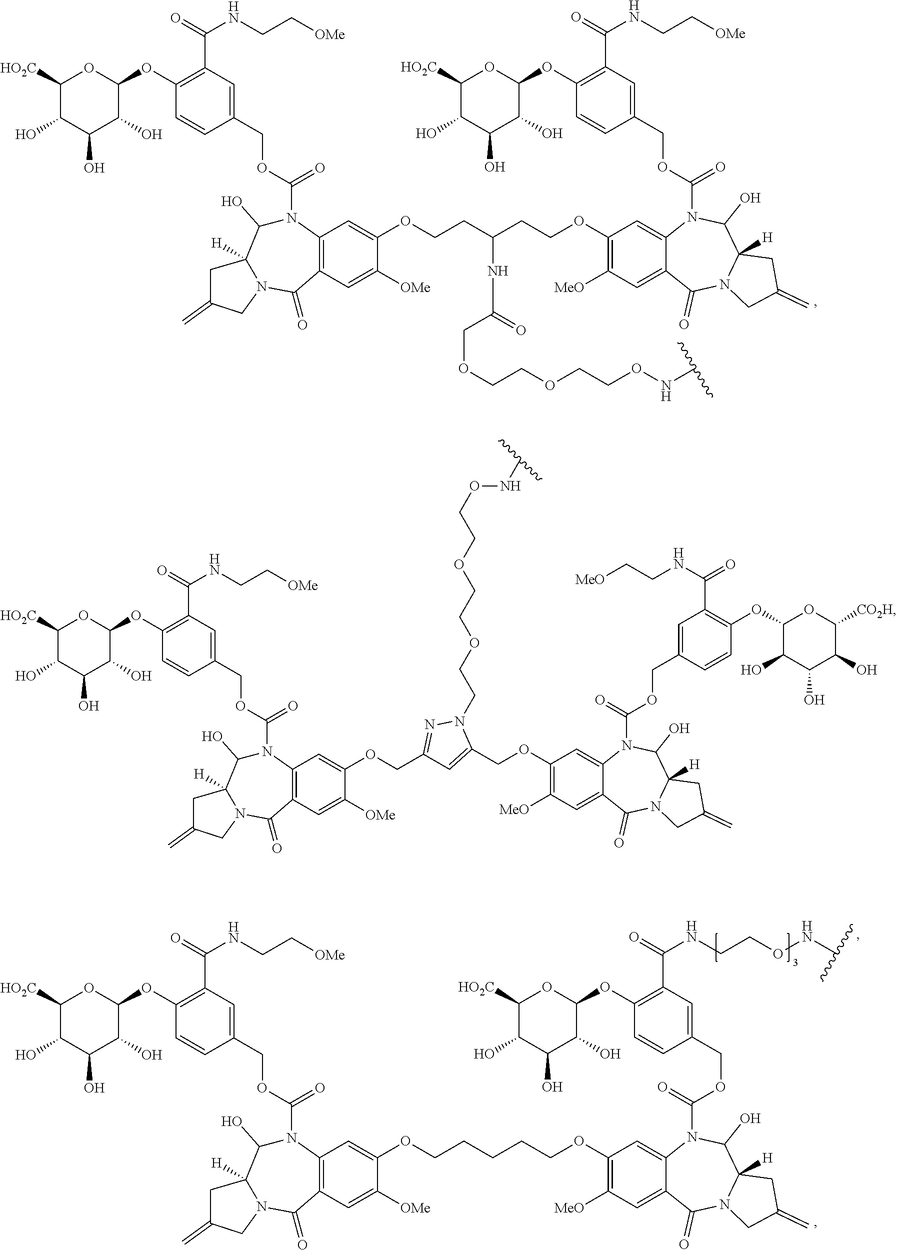

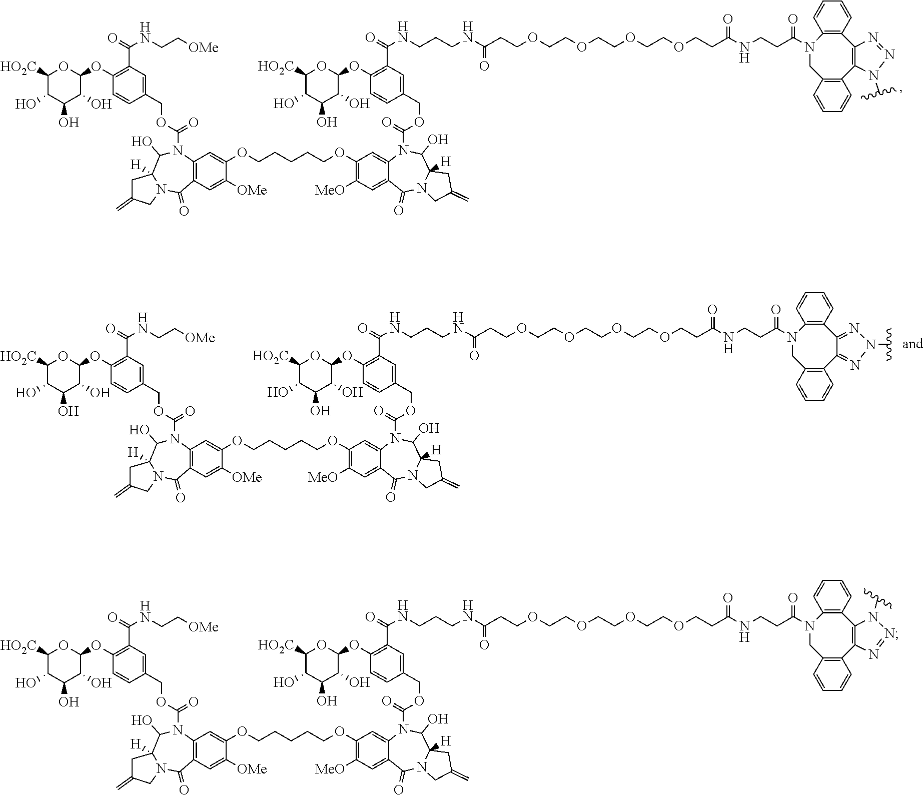

wherein MMAE is monomethyl auristatin E;
MMAF is monomethyl auristatin F; and
the wavy line is a connection point to the conjugate.

In certain aspects, the present disclosure also provides pharmaceutical compositions for the prevention or treatment of hyperproliferation, cancer, or an angiogenic disease, including the conjugate.

In certain embodiments, the pharmaceutical composition further comprises a pharmaceutically effective amount of a chemotherapeutic agent.

In certain embodiments, the cancer is selected from lung cancer, small cell lung cancer, gastrointestinal cancer, colon cancer, bowel cancer, breast cancer, ovarian cancer, prostate cancer, testicular cancer, liver cancer, kidney cancer, bladder cancer, pancreatic cancer, brain cancer, sarcoma, osteosarcoma, Kaposi [symbol] sarcoma, and melanoma.

The present disclosure also provides a pharmaceutical preparation comprises the conjugate.

### Antibody or Antigen-Binding Fragment Thereof

The present disclosure provides an antibody that binds to human TROP2 (tumor-associated calcium signal transducer 2, TACSTD2). As described herein, the antibody according to the present disclosure is a polypeptide comprising one or more complementarity-determining areas or regions (CDRs).

In some embodiments, the CDR is included in a “framework” region, and the framework orients the CDR(s) so that the CDR(s) can have appropriate antigen-binding properties.

In certain aspects, the present disclosure provides antibody-drug conjugates comprising an anti-TROP2 antibody that binds to TROP2. In certain embodiments, the antibody disclosed herein binds to TROP expressed in a tumor and may be used to deliver a drug to the tumor. In certain embodiments, the antibody drug conjugates disclosed herein have improved stability as compared to antibody drug conjugates known in the art.

In certain embodiments, the antibody comprises, but is not limited to, a monoclonal antibody, a bispecific antibody, a diabody, a multispecific antibody, a polyantibody, a minibody, a domain antibody, an antibody mimetic (or synthetic antibody), a chimeric antibody, a humanized antibody, a human antibody or an antibody fusion (or antibody conjugate), and a fragment thereof, and includes various forms of antibodies disclosed herein.

In certain embodiments, an antibody fragment of the antibody according to the present disclosure includes Fab, Fab [symbol] F(ab [symbol] , scFab, Fv, dsFv, scFV, scFV-Fc, a minibody, a diabody, scAb, or dAb.

In certain embodiments, the antibody according to the present disclosure may consist of a polypeptide of only light chains or only heavy chains including the variable regions shown in Tables 1 to 3.

CDR sequences that may be included in the heavy and light chain variable regions of the antibody or antigen-binding fragment thereof according to an embodiment of the present disclosure are shown in Tables 1 to 3, respectively.

An antibody according to the present disclosure shares certain regions or sequences with other antibodies disclosed herein. In certain embodiments, the constant region of the antibody or antigen-binding fragment thereof may be shared. In certain embodiments, Fc regions may be shared. In certain embodiment, the frame of a variable region may be shared.

The heavy chain variable region and the light chain variable region according to the present disclosure may be linked to at least a part of a human constant region. The selection of a constant region may be determined partially by whether or not antibody-dependent cell-mediated cytotoxicity, antibody-dependent cellular phagocytosis, and/or complement-dependent cytotoxicity is required. For example, human isotypes IgG1 and IgG3 have complement-dependent cytotoxicity, and human isotypes IgG2 and IgG4 do not have such cytotoxicity. In addition, human IgG land IgG3 induce a cell-mediated effector function stronger than that of human IgG2 and IgG4. The light chain constant region may be lambda or kappa.

A variable region of an immunoglobulin chain generally has the same overall structure and includes a comparatively conserved framework region (FR) linked by three hypervariable regions called “complementarity determining areas or regions or domains” or complementarity determining regions (CDRs). The CDRs of a variable region derived from each chain including a heavy chain/light chain pair are typically aligned by a framework region to form a structure specifically binding to a specific epitope of a target protein. These factors of naturally occurring light chain and heavy chain variable regions are typically included from the N-terminus to the C-terminus in the following order: FR1, CDR1, FR2, CDR2, FR3, CDR3, and FR4. The position of amino acid sequences corresponding to each variable region may be determined by Kabat (Kabat et al., (1983) U.S. Dept, of Health and Human Services, “Sequences of Proteins of Immunological Interest”), Chothia (Chothia and Lesk, J. Mol. Biol. 196:901-917 (1987)) or in a manner related to the OPAL library (Hye Young Yang et. al., 2009 Mol. Cells 27:225). The CDRs determined by each definition, when compared to each other, may be subsets which overlap or where one includes another. Those of ordinary skill in the art will be readily able to easily select CDR sequences according to the definitions above, given a variable region sequence of an antibody.

In certain embodiments, the antibody according to the present disclosure is a humanized antibody. A humanized antibody refers to any antibody in which the constant region of a non-human antibody is completely substituted with a human form of the constant region, and at least a portion of the variable region of a non-human antibody, except for the three loops of an amino acid sequence outside each variable region that binds to a target structure, is completely or partially substituted with the corresponding portion of a human antibody.

Certain mutations may be introduced to the framework region to enhance the stability of antibodies while maintaining their antigen binding activity. Stabilization of therapeutic antibodies can result in improved serum half-life, lower dosage requirements, reduced side-effects, improved shelf-life and reduced shipping and storage costs.

In certain embodiments, the humanized antibody according to the present disclosure comprises:

(a) (i) a variable heavy chain framework region from a heavy chain of a human antibody or from a human consensus framework; and (ii) a variable light chain framework region from a light chain of a human antibody or from a human consensus framework.

In certain embodiments, at least one amino acid of the variable domain framework region from the heavy chain is substituted with the corresponding amino acid from a heavy chain of a mouse antibody or mouse consensus framework; or the variable domain framework region from the light chain is substituted with the corresponding amino acid from the light chain of a mouse antibody or mouse consensus framework.

In certain embodiments, the amino acid substitution of the heavy chain is selected from the following amino acid substitutions:
substitution of Y (human) with F (mouse) at position 27;
substitution of T (human) with S (mouse) at position 30;
substitution of V (human) with L (mouse) at position 37;
substitution of M (human) with I (mouse) at position 48;
substitution of G (human) with A (mouse) at position 49;
substitution of I (human) with L (mouse) at position 70; and
substitution of R (human) with V (mouse) at position 72, and
(ii) the amino acid substitution of the light chain is substitution of Y (human) with S (mouse) at position 49.

In certain embodiments, the amino acid substitution of the heavy chain may further include the following amino acid substitution:
substitution of R (human) with K (mouse) at position 67; and/or
substitution of V (human) with A (mouse) at position 68.

In certain embodiments, the amino acid substitution of the light chain may further include the following amino acid substitution:
substitution of S (human) with Y (mouse) at position 67;
substitution of I (human) with T (mouse) at position 2 and substitution of S (human) with Y (mouse) at position 67;
substitution of T (human) with R (mouse) at position 22 and substitution of S (human) with Y (mouse) at position 67;
substitution of K (human) with E (mouse) at position 42 and substitution of S (human) with Y (mouse) at position 67;
substitution of G (human) with S (mouse) at position 64 and substitution of S (human) with Y (mouse) at position 67;
substitution of S (human) with Y (mouse) at position 67 and substitution of T (human) with V (mouse) at position 72; or
substitution of S (human) with Y (mouse) at position 67 and substitution of T (human) with D (mouse) at position 85.

In certain embodiments, the humanized antibody comprises: (a) (i) a variable heavy chain framework region from a heavy chain of a human antibody or from a human consensus framework, wherein the variable heavy chain framework region comprises one or more of the following amino acid sequence changes: Y27F, T30S, V37L, M48I, G49A, 170L, and R72V; and (ii) a variable light chain framework region from a light chain of a human antibody or from a human consensus framework, wherein the variable light chain framework region comprises the following amino acid sequence change: Y49S.

In certain embodiments, heavy chain and light chain variable region sequences including the substitutions are shown in Tables 1 to 3, respectively. In certain embodiments, the substitution includes substitution of the FR region shown in Tables 1 to 3.

In certain embodiments, the present disclosure discloses one or more amino acid sequences having substantial sequence identity to one or more amino acid sequences disclosed herein. Substantial identity means that the effects disclosed herein are maintained in the presence of sequence variations. In certain embodiments, the amino acid sequence has about 90% identity, about 95% identity, or about 99% identity to the heavy chain variable regions shown in Tables 1 to 3. In another embodiment, the amino acid sequence has about 90% identity, about 95% identity, or about 99% identity to the light chain variable regions shown in Tables 1 to 3. For example, in the case of variants exhibiting 90% identity, 95% identity, or 99% identity to the sequence of the antibody or antigen-binding fragment thereof according to the present disclosure, any mutation occurs in the framework of the variable region rather than the CDRs.

In certain embodiments, a nucleic acid encoding the antibody or fragment thereof according to the present disclosure is a nucleic acid encoding a full-length antibody including the CDRs disclosed herein, the variable region including the CDRs, and the variable region, and the constant region. Once the amino acid sequence is determined, the nucleic acid sequence may be easily determined in consideration of a known reverse transcription program, codon usage, and the like.

### Antigen Specificity and Affinity for Antibody

In certain preferred embodiments, the antibody or antigen-binding fragment thereof according to the present disclosure has specificity a human TROP2 antigen and affinity suitable for use as an antibody therapeutic/diagnostic agent. In certain embodiments, the affinity for aggregates may be KD<1,000 nM, <100 nM, <10 nM, <1 nM, <0.1 nM, <0.01 nM, or <0.001 nM, and may be, for example, 10⁻⁶ M to 10⁻¹² M.

### Production of Antibody

In the present disclosure, the non-human antibody may be derived from, for example, any antibody-producing animal, for example, a mouse, a rat, a rabbit, a goat, a donkey, or non-human primates (e.g., monkeys such as cynomolgus or rhesus monkey) or apes (e.g., chimpanzees). A non-human antibody may be produced by immunizing an animal by using a method known in the art. For a method for producing a humanized antibody, according to an embodiment of the present disclosure, refer to U.S. patent Ser. No. 15/532,598. A fully human antibody may be produced by administering an antigen to a transformed animal including a human immunoglobulin gene locus, or by treating a phage display library expressing a human antibody repertory with an antigen, and then selecting the target antibody. The antibody may be polyclonal or monoclonal, or may be synthesized within a cell host through the expression of recombinant DNA.

A monoclonal antibody (mAb) may be produced using a conventional monoclonal antibody method, for example, a standard somatic hybridization technique in the literature (see: Kohler and Milstein, 1975, Nature 256:495).

### Method for Expressing Antibody

The antibody disclosed herein may be expressed in a hybridoma cell line or an expression cell line other than a hybridoma. An expression construct encoding the antibody may be used to transform a mammalian, an insect or a microbial host cell. A construct such as a plasmid may be produced, as described in the foregoing description, using any of various known methods for introducing a polynucleotide into a host cell. The specific method may vary according to the type of host cell. Methods for introducing a heterogeneous polynucleotide into a mammalian cell are widely known in the art, and include, but are not limited to, for example, dextran-mediated transfer, calcium phosphate precipitation, polybrene-mediated transfer, protoplast fusion, electrophoresis, capsulation of a transferred polynucleotide using liposomes, mixing of a nucleic acid and a positively charged lipid, and direct microinjection of DNA into the nucleus.

### Use of Anti-Human TROP2 Antibody Drug-Conjugates for Therapeutic Purposes

The expression of TROP2 in cancer is associated with unfavorable prognosis of cancer patients and is known to also affect cancer metastasis. For example, TROP2 is overexpressed in lung cancer, small cell lung cancer, gastrointestinal cancer, colon cancer, bowel cancer, breast cancer, ovarian cancer, prostate cancer, testicular cancer, liver cancer, kidney cancer, bladder cancer, pancreatic cancer, brain cancer, sarcoma, osteosarcoma, Kaposi's sarcoma, melanoma, and the like. For anticancer antibody treatment, for example, the anti-human TROP2 antibody may, as described herein, be used in a form linked to various cytotoxic agents via a linker to remove TROP2-overexpressing cancer cells. Accordingly, an antibody binding to TROP2 may be used alone or in a form bonded to an anticancer chemotherapeutic agent, a cytotoxic agent or a radioactive material, or may be embodied as a cytotherapeutic agent such as CAR-T cells to target an anticancer target, and thus can be used as a targeted therapeutic agent for directing to TROP2-expressing cells.

### Treatment Method: Pharmaceutical Formulation and Administration Route

An embodiment of the present disclosure also provides a treatment method using an antibody-drug conjugate or a pharmaceutically acceptable salt or solvate thereof. In certain embodiments, the antibody-drug conjugate or pharmaceutically acceptable salt or solvate thereof is provided to a patient. The antibody-drug conjugate or pharmaceutically acceptable salt or solvate thereof inhibits cancer cell metastasis by binding to human TROP2 expressed on the surface of cancer cells. In certain embodiments, the antibody binds to human TROP2 expressed on the surface of cancer cells in a form bonded to a cytotoxic agent, thereby specifically delivering the cytotoxic agent bonded to the antibody to cancer cells, to induce the death of the cancer cells. In certain embodiments, the antibody binds to human TROP2 expressed on the surface of cancer cells in the form of an antibody specific to the same target or another target, thereby increasing specificity of multiple antibodies for cancer cells or inducing connections between cancer cells and other types of cells such as immune cells, to induce the death of the cancer cells.

Provided is also a pharmaceutical composition including a therapeutically effective amount of the antibody-drug conjugate or pharmaceutically acceptable salt or solvate thereof, and a pharmaceutically acceptable diluent, a carrier, a solubilizer, an emulsifier, a preservative, and/or an adjuvant. Also, for example, a method of treating a cancer patient by administering such a pharmaceutical composition is provided. The term “patient” includes human patients.

The pharmaceutical composition may include a pharmaceutically acceptable carrier. The carrier is used as a meaning including an excipient, a diluent, or an adjuvant. The carrier may be selected from the group consisting of, for example, lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinyl pyrrolidone, water, saline, buffer such as PBS, methylhydroxy benzoate, propylhydroxy benzoate, talc, magnesium stearate, and mineral oil. The composition may include a filler, an anticoagulant, a lubricant, a wetting agent, a flavoring agent, an emulsifier, a preservative, or a combination thereof.

The pharmaceutical composition may be prepared as any formulation according to general methods. The composition may be formulated into formulations for oral administration (e.g., powders, tablets, capsules, syrups, pills or granules) or for parenteral administration (for example, injections). In addition, the composition may be prepared as a systemic or local formulation.

The pharmaceutical composition may include an effective amount of the antibody or antigen-binding fragment thereof, an anticancer agent, or a combination thereof. The term “effective amount” refers to an amount sufficient to exhibit preventive or therapeutic effects when administered to an individual requiring prevention or treatment. The effective amount may be appropriately selected depending on a cell or individual that is selected by those or ordinary skill in the art. The effective amount may be determined according to factors including the severity of the disease, the age, body weight, health and gender of a patient, sensitivity of a patient to the drug, administration time, administration routes, excretion rate, treatment period, and drugs used in combination or simultaneously with the used composition, and other factors well known in the medical field.

The dosage of the pharmaceutical composition may range, for example, from 10 μg/kg to about 30 mg/kg, optionally from 0.1 mg/kg to about 30 mg/kg, or alternatively from 0.3 mg/kg to about 20 mg/kg per adult. The pharmaceutical composition may be administered once a day, multiple times a day, once every 1 to 4 weeks, or once to 12 times a year.

Hereinafter, the present disclosure will be described in more detail with reference to examples and experimental examples.

The following examples and experimental Examples are intended to aid in understanding of the present disclosure and are not intended to limit the scope of the present disclosure.

### Definitions

Unless otherwise defined in the present disclosure, scientific and technical terms used herein have the meaning as commonly understood by those of ordinary skill in the art. Furthermore, unless the context specifically requires otherwise, the singular includes the plural and the plural includes the singular. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N. Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skill in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

The term “conjugates” as used herein refers to cell binding agents that are covalently bonded to one or more molecules of a cytotoxic compound. In this regard, “cell binding agent” is a molecule having affinity for a biological target, and may be, for example, an antibody, particularly a monoclonal antibody, or an antibody fragment, and the binding agent functions to direct a biologically active compound to a biological target. In certain embodiments, the conjugate may be designed to target tumor cells through cell surface antigens. The antigen may be a cell surface antigen that is overexpressed or expressed in an abnormal cell type. Specifically, the target antigen may be expressed only on proliferative cells (e.g., tumor cells). The target antigen may be selected on the basis of different expression, usually between proliferative tissues and normal tissues. In the present disclosure, the antibody is bonded to the linker.

In the present disclosure, a “variant” of a polypeptide, for example, an antigen-binding fragment, a protein, or an antibody, is a polypeptide in which insertion, deletion, addition, and/or substitution have occurred at one or more amino acid residues compared to other polypeptide sequences, and includes fusion polypeptides. Protein variants also include those modified by protein enzymatic cleavage, phosphorylation or other post-translational modifications, but retaining the biological activity of the antibody disclosed herein, such as binding and specificity to ROR1. Variants may have about 99% identity, about 98% identity, about 97% identity, about 96% identity, about 95% identity, about 94% identity, about 93% identity, about 92% identity, about 91% identity, about 90% identity, about 89% identity, about 88% identity, about 87% identity, about 86% identity, about 85% identity, about 84% identity, about 83% identity, about 82% identity, about 81% identity, or about 80% identity to the sequence of the antibody or antigen-binding fragment thereof according to the present disclosure. Percent identity (%) or homology may be calculated by methods known in the art.

In certain embodiments, the percent homology or identity can be calculated by 100×[(same position)/min (TGA, TGB)], wherein TGA and TGB are the sum of the number of residues and internal gap positions in sequences A and B to be compared (Russell et al., J. Mol Biol., 244:332-350 (1994).

In the present disclosure, a conservative amino acid substitution refers to a substitution that does not substantially affect the activity or antigen line of a polypeptide. A polypeptide may include one or more conservative substitutions. Non-limiting examples thereof are shown in Table 3 below.

The term “derivative” of a polypeptide as used herein refers to a polypeptide that has chemical modification at one or more residues through conjugation with other chemical moieties, different from insertion, deletion, addition or substitution variants.

The term “naturally occurring” as used herein in relation to polypeptides, nucleic acids, host cells, and the like refers to substances that exist naturally.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site (at world wide web at blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at world wide web at ebi.ac.uk/Tools/psa.

As used herein, “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

In the present disclosure, “affinity” is the strength of interactions between an antibody or antigen-binding fragment thereof and an antigen, and is determined by properties of the antigen such as size, shape and/or charge of the antigen, and CDR sequences of the antibody or antigen-binding fragment thereof. The methods for determining the affinity are known in the art, and the following may be used as references.

The antibody or antigen-binding fragment thereof is called “specifically binding” to its target such as an antigen, when a dissociation constant (KD) is ≤10⁻⁶ M. The antibody specifically binds to a target with “high affinity” when KD is ≤1×10⁻⁸ M.

As used in the present disclosure, the “antigen-binding fragment” of a chain (heavy chain or light chain) of an antibody or immunoglobulin includes a part of an antibody which lacks some amino acids compared to a full-length chain, but can specifically bind to an antigen. This fragment can be considered as having biological activity, in that the fragment can specifically bind to a target antigen, or can compete with other antibodies or antigen binding fragments thereof to bind to a specific epitope. In certain embodiments, such a fragment includes at least one CDR present in a full-length light chain or heavy chain, and in some embodiments, includes a short-chain heavy chain and/or light chain, or part thereof. This biological active fragment may be produced by a recombinant DNA technique or may be produced, for example, by cleaving an intact antibody enzymatically or chemically. An immunologically functional immunoglobulin fragment includes, but is not limited to, Fab, Fab, F(ab)2, scFab, dsFv, Fv, scFV, scFV-Fc, diabody, minibody, 73cab, and dAb, and may be derived from any mammal, including, but not being limited to, a human, a mouse, a rat, a camelid, or a rabbit. The functional parts of antibodies such as the one or more CDRs disclosed in the present disclosure may be linked with a secondary protein or a small compound by a covalent bond, and thereby used as a targeted therapeutic agent for a specific target.

In the present disclosure, the “Fc” region includes two heavy chain fragments including CH2 and CH3 domains of an antibody. These two heavy chain fragments are linked to each other by hydrophobic interaction of two or more of disulfide bonds and a CH3 domain.

In the present disclosure, the “Fab fragment” consists of one light chain and one heavy chain including a variable region and CH1 only. The heavy chain of a Fab molecule cannot form a disulfide bond with another heavy chain molecule. In an scFab, two molecules of Fab are linked by a flexible linker.

In the present disclosure, the “Fab′ fragment” includes a Fab fragment and additionally a region between CH1 and CH2 domains of a heavy chain. A disulfide bond may form between two heavy chains of Fab′ fragments of two molecules, forming a F(ab′)2 molecule.

In the present disclosure, as described above, the “F(ab′)2 fragment” includes two light chains and two heavy chains including a variable region CH1 and part of a constant region between the CH1 and CH2 domains, with an inter-chain disulfide bond formed between the two heavy chains. Accordingly, a F(ab′)2 fragment consists of two Fab′ fragments, and the two Fab′ fragments are joined to each other by the disulfide bond therebetween.

In the present disclosure, the “Fv region” is a fragment of an antibody which includes each variable region of a heavy chain and a light chain, but does not include constant regions. In an sdFV, a heavy chain and a light chain are linked by a disulfide bond. In an scFc, the Fv is linked by a flexible linker. In an scFv-Fc, an Fc is linked to an scFV. In a minibody, CH₃ is linked to an scFV. A diabody includes the scFVs of two molecules.

In the present disclosure, the “single chain Fv” or “scFv” antibody fragment includes the VH and VL domains of an antibody, and these domains are present within a single polypeptide chain. An Fv polypeptide may additionally include a polypeptide linker between a Vh domain which enables the scFv to form the target structure for antigen binding, and a VL domain.

In the present disclosure, the “short-chain antibody (74cab)” is a single polypeptide chain including one constant region of a heavy chain or a light chain constant region in which heavy chain and light chain variable regions are linked by a flexible linker. For a short-chain antibody, U.S. Pat. No. 5,260,203 may be referred to, and short-chain antibody is disclosed herein by reference.

In the present disclosure, the “domain antibody (dAb)” is an immunologically functional immunoglobulin fragment including only a variable region of a heavy chain or a variable region of a light chain. In certain embodiments, two or more VH regions are linked by a covalent bond via a peptide linker, to form a bivalent domain antibody. Two VH regions of this bivalent domain antibody may target the same or different antigens.

In the present disclosure, “complementarity determining region” (CDR; that is, CDR1, CDR2, and CDR3) denotes amino acid residues of the variable domain of an antibody, which are necessary for binding to antigen. Each variable domain typically has three CDR domains, identified as CDR1, CDR2, and CDR3.

In the present disclosure, the “framework region” (FR) is a variable domain residue other than the CDR residues. Each variable domain typically has four FRs, identified as FR1, FR2, FR3, and FR4.

In the present disclosure, the “bivalent antigen-binding protein” or “bivalent antibody” includes two antigen-binding sites. The two antigen-binding sites included in a bivalent antibody may have the same antigen specificity, or may be a bispecific antibody where the antigen-biding sites bind to different antigens.

In the present disclosure, the “multispecific antigen-binding protein” or “multispecific antibody” targets two or more antigens or epitopes.

In the present disclosure, “linker” refers to a compound which covalently bonds a cytotoxic compound to an antibody.

In the present disclosure, “unsubstituted or substituted” is used to refer to a parent group which may be unsubstituted or substituted, “substituted” refers to a parent group having at least one substituent, and a substituent refers to a chemical moiety covalently bonded to or fused with a parent group.

In the present disclosure, “halo” refers to fluorine, chlorine, bromine, iodine, and the like.

In the present disclosure, “alkyl” is a monovalent moiety obtained by removing a hydrogen atom from a carbon atom of an aliphatic or alicyclic, saturated or unsaturated (unsaturated, fully unsaturated) hydrocarbon compound. As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted. Furthermore, the term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer. Examples of saturated alkyls may include methyl, ethyl, propyl, butyl, pentyl, hexyl, heptyl, and the like, examples of saturated linear alkyls may include methyl, ethyl, n-propyl, n-pentyl (amyl), n-hexyl, n-heptyl, and the like, examples of saturated branched cyclic alkyls may include isopropyl, isobutyl, sec-butyl, tert-butyl, isopentyl, neopentyl, and the like.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O) NH—.

The term “acyloxy” is art-recognized and refers to a group represented by the general formula hydrocarbylC(O)O—, preferably alkylC(O)O—.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amido”, as used herein, refers to a group

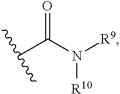

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

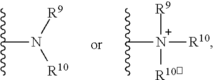

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “alkoxy” as used herein refers to —OR where R is an alkyl group, and examples thereof may include methoxy, ethoxy, n-propoxy, isopropoxy, n-butoxy, sec-butoxy, isobutoxy, tert-butoxy, and the like.

The term “alkenyl” as used herein is an alkyl having at least one carbon-carbon double bond. Examples of unsaturated alkenyl groups may include ethenyl (vinyl, —CH═CH₂), 1-propenyl (—CH═CHCH₃), 2-propenyl, isopropenyl, butenyl, pentenyl, and hexenyl.

In the present disclosure, the term “alkynyl” as used herein refers to an alkyl group having at least one carbon-carbon triple bond, and examples of unsaturated alkynyl group may include ethynyl and 2-propynyl.

The term “carboxy” as used herein refers to —C(═O) OH.

The term “formyl” as used herein refers to —C(═O) H.

The term “aryl” as used herein refers to a monovalent moiety obtained by removing a hydrogen atom from an aromatic ring atom of an aromatic compound. For example, the term “C₅₋₇ aryl” refers to a moiety having 5 to 7 ring atoms, which is a monovalent moiety obtained by removing a hydrogen atom from the aromatic ring atom of an aromatic compound, and the term “C₅₋₁₀ aryl” refers to a moiety having 5 to 10 ring atoms, which is a monovalent moiety obtained by removing a hydrogen atom from the aromatic ring atom of an aromatic compound. In this regard, the prefixes (C₅₋₇, C₅₋₁₀, and the like) refer to the number of ring atoms or a range of the number of ring atoms, regardless of whether they are carbon atoms or hetero atoms. For example, the term “C₅₋₆ aryl” refers to an aryl group having 5 or 6 ring atoms. In this regard, the ring atoms may be all carbon atoms as in a “carboaryl group.” Examples of carboaryl groups include, but are not limited to, those derived from benzene, naphthalene, azulene, anthracene, phenanthrene, naphthacene, and pyrene. Examples of aryl groups containing fused rings wherein at least one is an aromatic ring include, but are not limited to, groups derived from indane, indene, isoindene, tetralin, acenaphthene, fluorene, phenalene, acephenanthrene, and aseantrene. Alternatively, the ring atoms may contain one or more heteroatoms as in a “heteroaryl group.”

The term “carbamate” is art-recognized and refers to a group

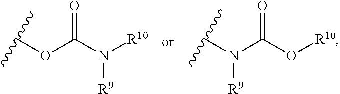

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbonate” is art-recognized and refers to a group —OCO₂—.

The term “carboxy”, as used herein, refers to a group represented by the formula-CO₂H.

The term “cycloalkyl” as used herein refers to an alkyl group which is a cyclyl group, and relates to a monovalent moiety obtained by removing a hydrogen atom from an alicyclic ring atom of a cyclic hydrocarbon compound. Examples of cycloalkyl groups include, but are not limited to, those derived from: saturated single ring hydrocarbon compounds, such as cyclopropane, cyclobutane, cyclopentane, cyclohexane, cycloheptane, methylcyclopropane, dimethylcyclopropane, methylcyclobutane, dimethylcyclobutane, methylcyclopentane, dimethylcyclopentane, and methylcyclohexane; or unsaturated single ring hydrocarbon compounds, such as cyclopropene, cyclobutene, cyclopentene, cyclohexene, methylcyclopropene, dimethylcyclopropene, methylcyclobutene, dimethylcyclobutene, methylcyclopentene, dimethylcyclopentene, and methylcyclohexene; and saturated heterocyclic hydrocarbon compounds, such as norcaran, norphenen, and norbornene.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The term “halo” or “halogen” used herein refers to fluoro (—F), chloro (—Cl), bromo (—Br), and iodo (—I).

The terms “heteroalkyl” and “heteroaralkyl”, as used herein, refers to an alkyl group substituted with a hetaryl group.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The term “heteroaryl” as used herein refers to aryl containing one or more heteroatoms, and examples thereof may include pyridine, pyrimidine, benzothiophene, furyl, dioxolanyl, pyrrolyl, oxazolyl, pyridyl, pyridazinyl, and pyrimidinyl. More specifically, examples thereof may include benzofuran, isobenzofuran, indole, isoindole, indolizine, indolin, isoindoline, purine (adenine or guanine), benzimidazole, indazole, benzoxazole, benzisoxazole, benzodioxole, benzofuran, benzotriazole, benzothiofuran, benzothiazole, C₉ having two fused rings derived from benzothiazole, chromene, iso-chromene, chroman, iso-chroman, benzodioxane, quinoline, isoquinoline, quinolizine, benzoxazine, benzodiazine, pyridopyridine, quinoxaline, quinazoline, cinnoline, phthalazine, naphthyridine, C₁₀ having two fused rings derived from pteridin, C₁₁ having two fused rings derived from benzodiazepine, carbazole, dibenzofuran, dibenzothiophene, carboline, pyrimidine, C₁₃ having three fused rings derived from pyridoindole, acridine, xanthene, thioxanthene, oxanthrene, phenoxathiin, phenazine, phenoxazine, phenothiazine, thianthrene, phenanthridine, phenanthroline, and C₁₄ having three fused rings derived from phenazine.

The term “heterocyclyl” as used herein refers to a monovalent moiety obtained by removing a hydrogen atom from a ring atom of a heterocyclic compound. Furthermore, the term “heterocyclyl” or “heterocycle” used herein refers to a monosaturated or partially unsaturated non-aromatic compound or non-aromatic multi-ring system in which at least one heteroatom (that is, at least one cyclic heteroatom selected from oxygen, nitrogen and sulfur) is included in the ring. Unless otherwise specified, heterocyclyl groups have 5 to about 20 ring atoms, such as 3 to 12 ring atoms, such as 5 to 10 ring atoms. Thus, the term includes a single saturated or partially unsaturated ring (for example, 3, 4, 5, 6 or 7-membered rings), having about 1 to 6 cyclic carbon atoms and about 1 to 3 cyclic heteroatoms selected from oxygen, nitrogen and sulfur, in the ring. The rings of a multiple condensed ring system may be linked to one another through fusion, spiro and cross-linking bonds as long as valency requirements are satisfied. Examples of heterocycles include azetidine, aziridine, imidazolidine, morpholine, oxirane (epoxide), oxetane, piperazine, piperidine, pyrazolidine, piperidine, pyrrolidine, pyrrolidinone, tetrahydrofuran, tetrahydrothiophene, dihydropyridine, tetrahydropyridine, quinuclidine, N-bromopyrrolidine, N-chloropiperidine, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamido” is art-recognized and refers to the group represented by the general formulae

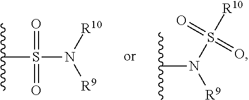

wherein R⁹ and R¹⁰ independently represents hydrogen or hydrocarbyl.

The term “sulfoxide” is art-recognized and refers to the group —S(O)—.

The term “sulfonate” is art-recognized and refers to the group —SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

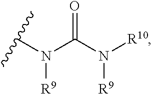

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

In the present disclosure, the prefixes (e.g., C₁₋₁₂, C₃₋₈, and the like) refer to the number of ring atoms or a range for the number of ring atoms, regardless of whether they are carbon atoms or hetero atoms. For example, the term “C₃₋₆ heterocyclyl” as used herein relates to a heterocyclyl group having 3 to 6 ring atoms.

The term “prodrug” as used herein refers to compounds which, under in vivo physiological conditions (e.g., enzymatic oxidation, reduction and/or hydrolysis), may be converted directly or indirectly into a pyrrolobenzodiazepine drug by action of an enzyme or gastric acid.

In the present disclosure, “pharmaceutically acceptable salt” may be an acid addition salt formed by pharmaceutically acceptable free acid, and an organic acid or an inorganic acid may be used as the free acid.

Examples of the organic acid include, but are not limited to, citric acid, acetic acid, lactic acid, tartaric acid, maleic acid, fumaric acid, formic acid, propionic acid, oxalic acid, trifluoroacetic acid, benzoic acid, gluconic acid, meta-sulfonic acid, glycolic acid, succinic acid, 4-toluenesulfonic acid, glutamic acid, and aspartic acid. Also, examples of the inorganic acid include, but are not limited to, hydrochloric acid, bromic acid, sulfuric acid, and phosphoric acid.

For example, when the compound has a functional group which is an anion or may be an inion (e.g., —COOH may be —COO—), the compound may form a salt with a suitable cation. Examples of suitable inorganic cations include, but are not limited to, alkali metal ions such as Na⁺ and K⁺, alkali earth metal cations such as Ca²⁺ and Mg²⁺, and other cations such as A13. Examples of suitable organic cations include, but are not limited to, include ammonium ions (i.e., NH₄⁺) and substituted ammonium ions (e.g., NH₃R⁺, NH₂R₂⁺, NHR₃⁺, and NR₄⁺).

Examples of some suitable substituted ammonium ions are those derived from the following: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids such as lysine and arginine. Examples of typical quaternary ammonium ions include N(CH₃)₄⁺.

When the compound has a functional group which is a cation or may be a cation (e.g., —NH₂ may be —NH₃⁺), the compound may form a salt with a suitable anion. Examples of suitable inorganic anions include, but are not limited to, those derived from the following inorganic acids: hydrochloric acid, hydrobromic acid, hydroiodic acid, sulfuric acid, sulfurous acid, nitric acid, nitrous acid, phosphoric acid, and phosphorous acid.

Examples of suitable organic anions include, but are not limited to, those derived from the following organic acids: 2-acetioxybenzoic acid, acetic acid, ascorbic acid, aspartic acid, benzoic acid, camphorsulfonic acid, cinnamic acid, citric acid, edetic acid, ethanedisulfonic acid, ethanesulfonic acid, fumaric acid, gluteptonic acid, gluconic acid, glutamic acid, glycolic acid, hydroxymaleic acid, hydroxynaphthalene carboxylic acid, isethionic acid, lactic acid, lactobionic acid, lauric acid, maleic acid, malic acid, methansulfonic acid, mucoic acid, oleic acid, oxalic acid, palmitic acid, palmic acid, pantothenic acid, phenylacetic acid, phenylsulfonic acid, propionic acid, pyruvic acid, salicylic acid, stearic acid, succinic acid, sulfanilic acid, tartaric acid, toluenesulfonic acid, and valeric acid. Examples of suitable polymeric organic anions include, but are not limited to, those derived from the following polymeric acids: tannic acid and carboxymethyl cellulose.

The term “solvate” as used herein refers to a molecular complex between a compound according to the present disclosure and solvent molecules, and examples thereof include, but are not limited to, water, isopropanol, ethanol, methanol, dimethylsulfoxide, ethyl acetate, acetic acid, ethanolamine, or the compound according to the present disclosure bonded to a mixed solvent thereof.

It may be convenient or desirable to prepare, purify and/or handle the equivalent solvate of an active compound. The term “solvate” is used herein in its conventional sense to refer to a complex of a solute (e.g., an active compound or a salt thereof) and a solvent. When the solvent is water, the solvate may conveniently be referred to as a hydrate, such as monohydrate, dihydrate or trihydrate.

The phrase “effective amount” or “therapeutically effective amount” as used herein refers to an amount required to achieve a target therapeutic result (for administration amount and administration period and means). An effective dose is a minimum amount of activating agent necessary to give an object a minimum therapeutic benefit, and is less than a toxic dose. For example, a dosage may be in a range of about 100 ng to about 100 mg/kg per patient, more typically in a range of about 1 μg/kg to about 10 mg/kg. When the active compound is a salt, an ester, an amide, a prodrug, or the like, the dosage is calculated on the basis of a parent compound, and thus the actual weight used increases proportionally. The pyrrolobenzodiazepine compound according to the present disclosure may be formulated to include 0.1 mg to 3000 mg, 1 mg to 2000 mg, or 10 mg to 1000 mg of an active ingredient per unit dosage form, but the present disclosure is not limited thereto. The active ingredient may be administered to obtain a peak plasma concentration of the active compound of about 0.05 μM to about 100 μM, about 1 μM to about 50 ¶M, or about 5 μM to about 30 μM. For example, a 0.1 w/v % to 5 w/v % solution of the active ingredient in saline may be administered via an intravenous injection.

The concentration of the active compound in the pharmaceutical composition may be determined by the absorption, inactivation and excretion rate of the drug, and other factors known to those of ordinary skill in the art. The dosage may vary depending on the severity of symptoms/diseases. In addition, the dosage and administration method for a specific patient may be adjusted according to the professional judgment of an administration supervisor, in consideration of the severity of symptoms/diseases of patients, necessity, age, drug response, and the like, and the concentration range described herein is only an example and is not intended to limit the embodiments of the claimed composition. In addition, the active ingredient may be administered once, or a much smaller dosage of the active ingredient may also be administered in multiple doses.

The term “linker” as used herein refers to a compound that covalently bonds a cytotoxic compound to an antibody.

The terms “intracellularly cleaved” and “intracellular cleavage” as used herein refer to a metabolic process or reaction inside a cell on an antibody construct-active agent conjugate, by which the covalent attachment, e.g., the linker, between an active agent (B) and an antibody construct (Ab) is broken, thus causing a free drug or other metabolites of the conjugate dissociated from the antibody inside the cell.

### EXAMPLES

The invention now being generally described, it will be more readily understood by reference to the following examples which are included merely for purposes of illustration of certain aspects and embodiments of the present invention and are not intended to limit the invention.

### Example 1: Production of Antibody Specific to Human TROP2 (Tumor-Associated Calcium Signal Transducer 2, TACSTD2)

Anti-TROP2 humanized antibodies 2G10-5 and 2G10-6, which specifically bind more strongly to TROP2 (tumor-associated calcium signal transducer 2, TACSTD2) mainly expressed in tumors, were produced by the method described in U.S. patent Ser. No. 15/532,598. In addition, the amino acid sequences of the antibodies are shown in Tables 1 and 2 below.

TABLE 1

| | | SEQ ID |
|---|---|---|
| 2G10-5 heavy chain | | NO: |
| FR1 | QVQLVQSGAEVKKPGASVKVSCKASGFTFS | 1 |
| CDR1 | SSYIS | 2 |
| FR2 | WLRQAPGQRLEWIA | 3 |
| CDR2 | WIYAGTGGTSYNQKFTG | 4 |
| FR3 | KATLTVDTSASTAYMELSSLRSEDTAVYYCAR | 5 |
| CDR3 | HNPRYYAMDY | 6 |
| FR4 | WGQGTTVTVSS | 7 |
| VH | QVQLVQSGAEVKKPGASVKVSCKASGFTFSSSYISWLRQAPGQRLE | 15 |
| | WIAWIYAGTGGTSYNQKFTGKATLTVDTSASTAYMELSSLRSEDTA | |
| | VYYCARHNPRYYAMDYWGQGTTVTVSS | |
| HC | QVQLVQSGAEVKKPGASVKVSCKASGFTFSSSYISWLRQAPGQRLE | 17 |
| | WIAWIYAGTGGTSYNQKFTGKATLTVDTSASTAYMELSSLRSEDTA | |
| | VYYCARHNPRYYAMDYWGQGTTVTVSSASTKGPSVFPLAPSSKSTS | |
| | GGTAALGCLVKDYFPEPVTVSWNSGALTSGVHTFPAVLQSSGLYSL | |
| | SSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFN | |
| | WYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| | | SEQ ID |
| 2G10-5 light chain | | NO: |
| FR1 | DTQMTQSPSSLSASVGDRVTITC | 8 |
| CDR1 | ITSTDIDDDMN | 9 |
| FR2 | WYQQKPGKAPKLLIS | 10 |
| CDR2 | EGNTLRP | 11 |
| FR3 | GVPSRFSGSGYGTDFTFTISSLQPEDIATYYC | 12 |
| CDR3 | LQSDNLPYT | 13 |
| FR4 | FGGGTKVEIK | 14 |
| VL | DTQMTQSPSSLSASVGDRVTITCITSTDIDDDMNWYQQKPGKAPKL | 16 |
| | LISEGNTLRPGVPSRFSGSGYGTDFTFTISSLQPEDIATYYCLQSDNLP | |
| | YTFGGGTKVEIK | |
| LC | DTQMTQSPSSLSASVGDRVTITCITSTDIDDDMNWYQQKPGKAPKL | 18 |
| | LISEGNTLRPGVPSRFSGSGYGTDFTFTISSLQPEDIATYYCLQSDNLP | |
| | YTFGGGTKVEIKRTVAAPSVFIFPPSDEQLKSGTASVVCLLNNFYPRE | |
| | AKVQWKVDNALQSGNSQESVTEQDSKDSTYSLSSTLTLSKADYEK | |
| | HKVYACEVTHQGLSSPVTKSFNRGEC | |

TABLE 2

| | | SEQ ID |
|---|---|---|
| 2G10-6 heavy chain | | NO: |
| FR1 | QVQLVQSGAEVKKPGASVKVSCKASGFTFS | 1 |
| CDR1 | SSYIS | 2 |
| FR2 | WLRQAPGQRLEWIA | 3 |
| CDR2 | WIYAGTGGTSYNQKFTG | 4 |
| FR3 | RVTLTVDTSASTAYMELSSLRSEDTAVYYCAR | 19 |
| CDR3 | HNPRYYAMDY | 6 |
| FR4 | WGQGTTVTVSS | 7 |
| VH | QVQLVQSGAEVKKPGASVKVSCKASGFTFSSSYISWLRQAPGQRLE | 20 |
| | WIAWIYAGTGGTSYNQKFTGRVTLTVDTSASTAYMELSSLRSEDTA | |
| | VYYCARHNPRYYAMDYWGQGTTVTVSS | |
| HC | QVQLVQSGAEVKKPGASVKVSCKASGFTFSSSYISWLRQAPGQRLE | 21 |
| | WIAWIYAGTGGTSYNQKFTGRVTLTVDTSASTAYMELSSLRSEDTA | |
| | VYYCARHNPRYYAMDYWGQGTTVTVSSASTKGPSVFPLAPSSKSTS | |
| | GGTAALGCLVKDYFPEPVTVSWNSGALTSGVHTFPAVLQSSGLYSL | |
| | SSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| | | SEQ ID |
| 2G10-6 light chain | | NO: |
| FR1 | DTQMTQSPSSLSASVGDRVTITC | 8 |
| CDR1 | ITSTDIDDDMN | 9 |
| FR2 | WYQQKPGKAPKLLIS | 10 |
| CDR2 | EGNTLRP | 11 |
| FR3 | GVPSRFSGSGYGTDFTFTISSLQPEDIATYYC | 12 |
| CDR3 | LQSDNLPYT | 13 |
| FR4 | FGGGTKVEIK | 14 |
| VL | DTQMTQSPSSLSASVGDRVTITCITSTDIDDDMNWYQQKPGKAPKL | 16 |
| | LISEGNTLRPGVPSRFSGSGYGTDFTFTISSLQPEDIATYYCLQSDNLP | |
| | YTFGGGTKVEIK | |
| LC | DTQMTQSPSSLSASVGDRVTITCITSTDIDDDMNWYQQKPGKAPKL | 18 |
| | LISEGNTLRPGVPSRFSGSGYGTDFTFTISSLQPEDIATYYCLQSDNLP | |
| | YTFGGGTKVEIKRTVAAPSVFIFPPSDEQLKSGTASVVCLLNNFYPRE | |
| | AKVQWKVDNALQSGNSQESVTEQDSKDSTYSLSSTLTLSKADYEK | |
| | HKVYACEVTHQGLSSPVTKSFNRGEC | |

In addition, for ADC synthesis, an antibody clone 2G10-5-CaaX was constructed by fusing a CaaX peptide moiety (GGGGGGGGVIM; SEQ ID NO: 22) to the C-terminus of the light chain (SEQ ID NO: 18) of the antibody 2G10-5, and antibodies were produced through transient expression based on CHO cells. The produced antibody 2G10-5-CaaX was used for ADC synthesis. The amino acid sequences of the antibodies are shown in Table 3.

TABLE 3

| | | SEQ ID |
|---|---|---|
| 2G10-5-CaaX heavy chain | | NO: |
| FR1 | QVQLVQSGAEVKKPGASVKVSCKASGFTFS | 1 |
| CDR1 | SSYIS | 2 |
| FR2 | WLRQAPGQRLEWIA | 3 |
| CDR2 | WIYAGTGGTSYNQKFTG | 4 |
| FR3 | KATLTVDTSASTAYMELSSLRSEDTAVYYCAR | 5 |
| CDR3 | HNPRYYAMDY | 6 |
| FR4 | WGQGTTVTVSS | 7 |
| VH | QVQLVQSGAEVKKPGASVKVSCKASGFTFSSSYISWLRQAPGQRLE | 15 |
| | WIAWIYAGTGGTSYNQKFTGKATLTVDTSASTAYMELSSLRSEDTA | |
| | VYYCARHNPRYYAMDYWGQGTTVTVSS | |
| HC | QVQLVQSGAEVKKPGASVKVSCKASGFTFSSSYISWLRQAPGQRLE | 17 |
| | WIAWIYAGTGGTSYNQKFTGKATLTVDTSASTAYMELSSLRSEDTA | |
| | VYYCARHNPRYYAMDYWGQGTTVTVSSASTKGPSVFPLAPSSKSTS | |
| | GGTAALGCLVKDYFPEPVTVSWNSGALTSGVHTFPAVLQSSGLYSL | |
| | SSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFN | |
| | WYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPGK | |
| | | SEQ ID |
| 2G10-5-CaaX light chain | | NO: |
| FR1 | DTQMTQSPSSLSASVGDRVTITC | 8 |
| CDR1 | ITSTDIDDDMN | 9 |
| FR2 | WYQQKPGKAPKLLIS | 10 |
| CDR2 | EGNTLRP | 11 |
| FR3 | GVPSRFSGSGYGTDFTFTISSLQPEDIATYYC | 12 |
| CDR3 | LQSDNLPYT | 13 |
| FR4 | FGGGTKVEIK | 14 |
| VL | DTQMTQSPSSLSASVGDRVTITCITSTDIDDDMNWYQQKPGKAPKL | 16 |
| | LISEGNTLRPGVPSRFSGSGYGTDFTFTISSLQPEDIATYYCLQSDNLP | |
| | YTFGGGTKVEIK | |
| LC | DTQMTQSPSSLSASVGDRVTITCITSTDIDDDMNWYQQKPGKAPKL | 23 |
| | LISEGNTLRPGVPSRFSGSGYGTDFTFTISSLQPEDIATYYCLQSDNLP | |
| | YTFGGGTKVEIKRTVAAPSVFIFPPSDEQLKSGTASVVCLLNNFYPRE | |
| | AKVQWKVDNALQSGNSQESVTEQDSKDSTYSLSSTLTLSKADYEK | |
| | HKVYACEVTHQGLSSPVTKSFNRGECGGGGGGGCVIM | |

### Example 2: Synthesis Preparation of Compounds 1, 2, 3 and 4

### <2-1> Preparation of Compound 1

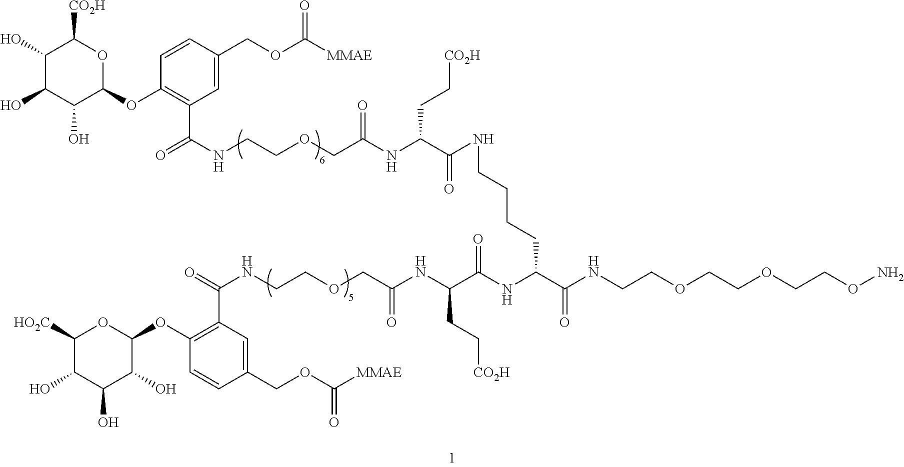

Compound 1 was prepared using the method described in Korean Patent Application No. 10-2018-7018068.

The structure of monomethyl auristatin E (MMAE) in compound 1 is as follows:

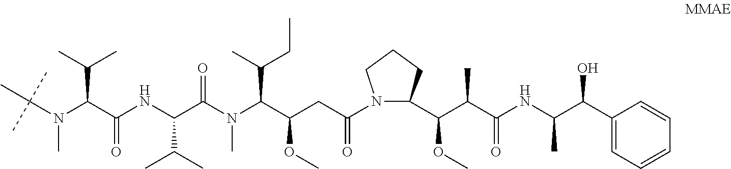

### <2-2> Preparation of Compound 2

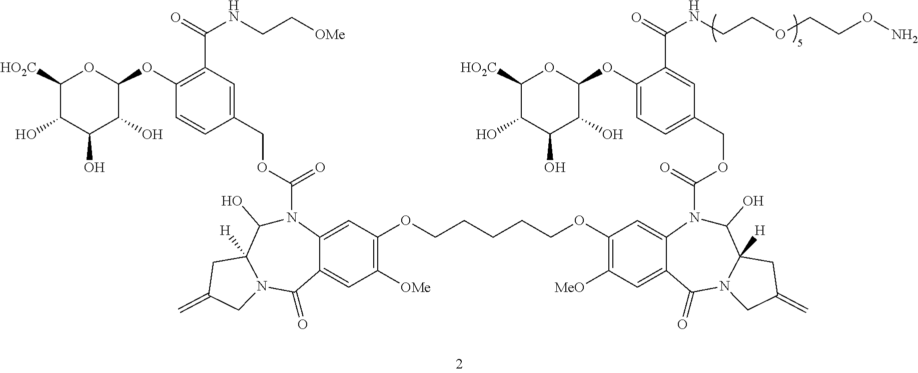

Compound 2 was prepared using the method described in Korean Patent Application No. 10-2018-0036895.

### <2-3> Preparation of Compound 3

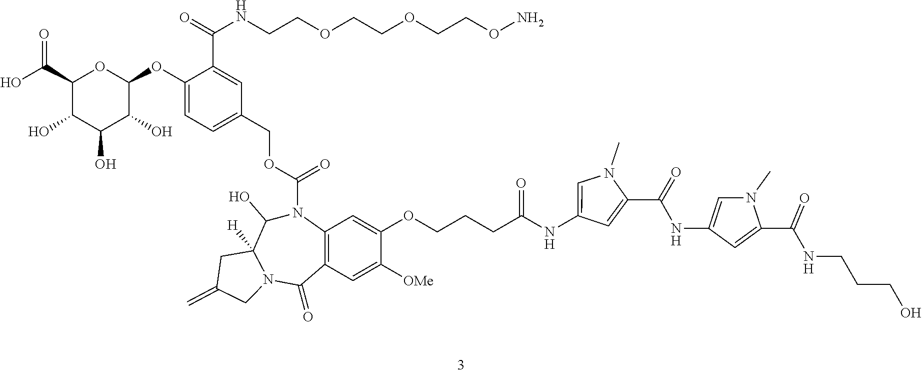 [SMILES: C=C1C[C@H]2C(O)N(C(=O)OCc3ccc(O[C@@H]4O[C@H](C(=O)O)[C@@H](O)[C@H](O)[C@H]4O)c(C(=O)NCCOCCOCCON)c3)c3cc(OCCCC(=O)Nc4cc(C(=O)Nc5cc(C(=O)NCCCO)n(C)c5)n(C)c4)c(OC)cc3C(=O)N2C1]

Compound 3 was prepared using the method described in Korean Patent Application No. 10-2020-0188314.

### <2-4> Preparation of Compound 4

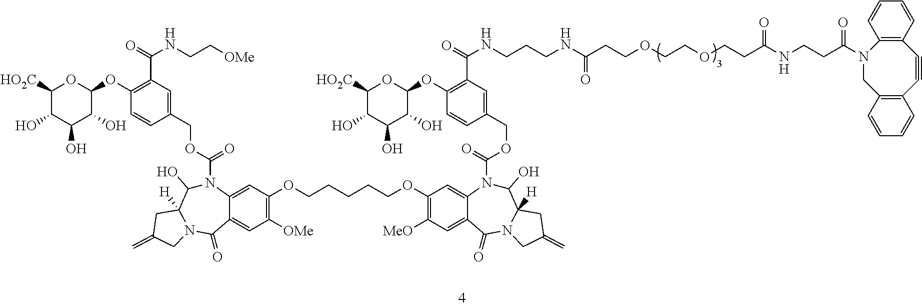

Compound 4 was prepared using the method described in Korean Patent Application No. 10-2018-0036895.

### Example 3: Synthesis Production of ADCs

ADCs were prepared through the following two steps, and LCB14-0606 and LCB14-0512 used commonly were prepared using the method described in Korean Patent Application No. 10-2013-7032628. The structural formulae of LCB14-0606 and LCB14-0512 are as follows:

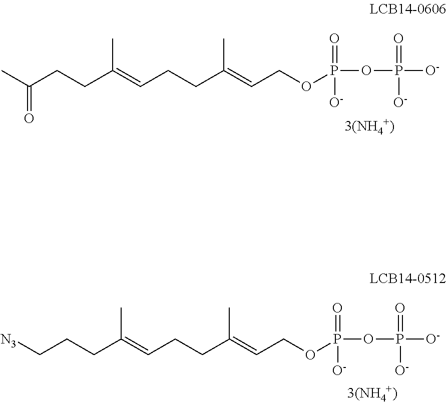 [SMILES: CC(=O)CC/C(C)=C/CC/C(C)=C/COP(=O)([O-])OP(=O)([O-])[O-] ; C/C(=C\COP(=O)([O-])OP(=O)([O-])[O-])CC/C=C(\C)CCCN=[N+]=[N-]]

### Step 1: Production of Prenylated Antibodies

### <Production of Prenylated Antibody Using LCB14-0606>

A prenylation reaction mixture of the antibody 2G10-5-CaaX of Example 1 was prepared and a reaction was allowed to occur at 30° C. for 16 hours. The reaction mixture consisted of a 24 μM antibody, 600 nM FTase (Genscript), and a buffer solution (50 mM Tris-HCl (pH 7.4), 5 mM MgCl₂, 10 μM ZnCl₂, and 0.5 mM DTT) containing 144 μM LCB14-0606. After the reaction was completed, the prenylated antibody was desalted using a G25 Sepharose column (AKTA purifier, GE healthcare) equilibrated with PBS buffer solution.

### <Production of Prenylated Antibody Using LCB14-0512>

A prenylation reaction mixture of the antibody 2G10-5-CaaX of Example 1 was prepared and a reaction was allowed to occur at 30° C. for 2 hours. The reaction mixture consisted of a 24 μM antibody, 600 nM FTase (Genscript), and a buffer solution (50 mM Tris-HCl (pH 7.4), 5 mM MgCl₂, 10 μM ZnCl₂, and 0.1 mM DTT) containing 200 μM LCB14-0512. After the reaction was completed, the prenylated antibody was desalted using a G25 Sepharose column (AKTA purifier, GE healthcare) equilibrated with PBS buffer solution.

### Step 2: Drug Conjugation Method

### <Conjugation by Oxime Bond Formation)>

The mixture for oxime bond-formation reaction between the prenylated antibody and linker-drug was prepared by mixing 100 mM Na-acetate buffer solution pH 5.2, 10% DMSO, 24 UM of the prenylated antibody, and 240 μM linker-drug (in house, compound 1, 2, or 3 of Example 2), and stirred lightly at 30° C. After reacting for 6 hours or 24 hours, an FLPC (AKTA purifier, GE healthcare) process was carried out to remove the excess of small compounds used, and the protein fraction was collected and concentrated.

### <Conjugation by Copper-Free Click Reaction>

For the production of an ADC using the copper-free click coupling reaction between the prenylated antibody and linker-drug, PBS buffer solution (pH 7,4), 1% DMSO, 10 UM of the prenylated antibody, and 100 μM linker-drug (in house, compound 4 of Example 2) were mixed to prepare a reaction mixture, and after reaction at 25° C. for 6 hours, the excess of small-molecule compounds used were removed through FPLC (AKTA purifier, GE healthcare) or a G25 Sepharose column process, and the protein fraction was collected concentrated.

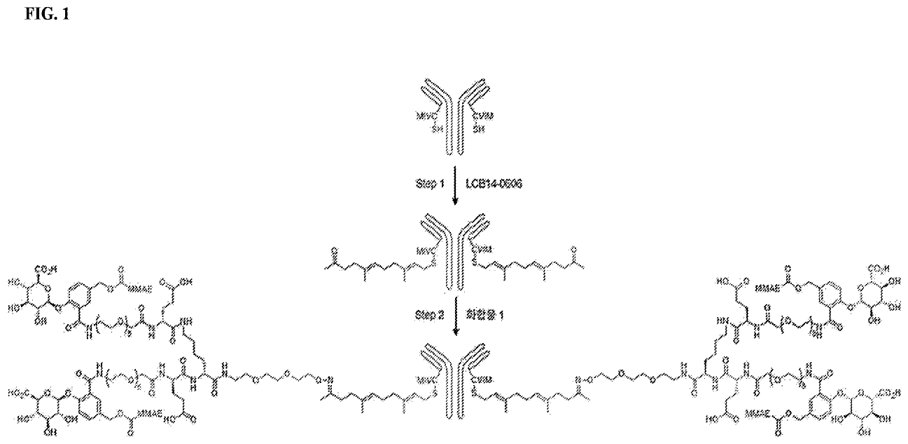

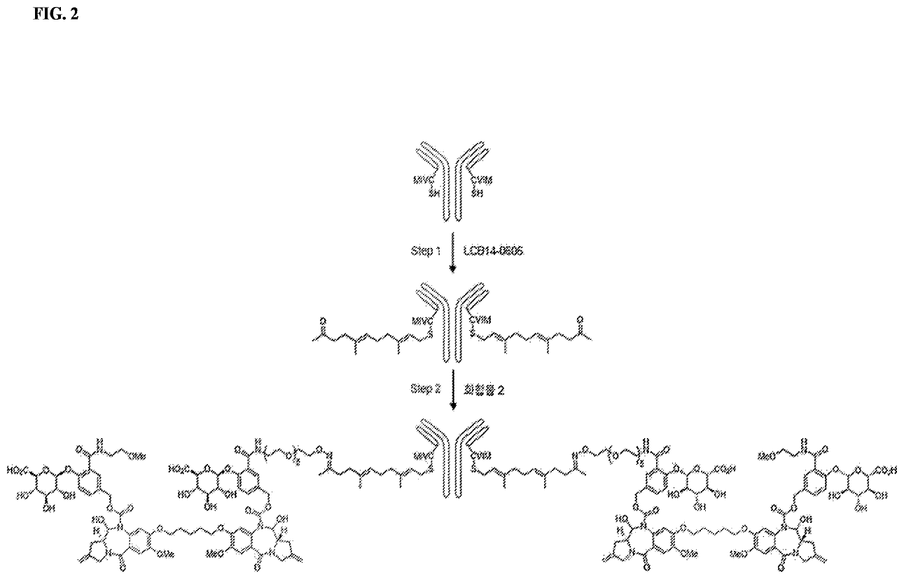

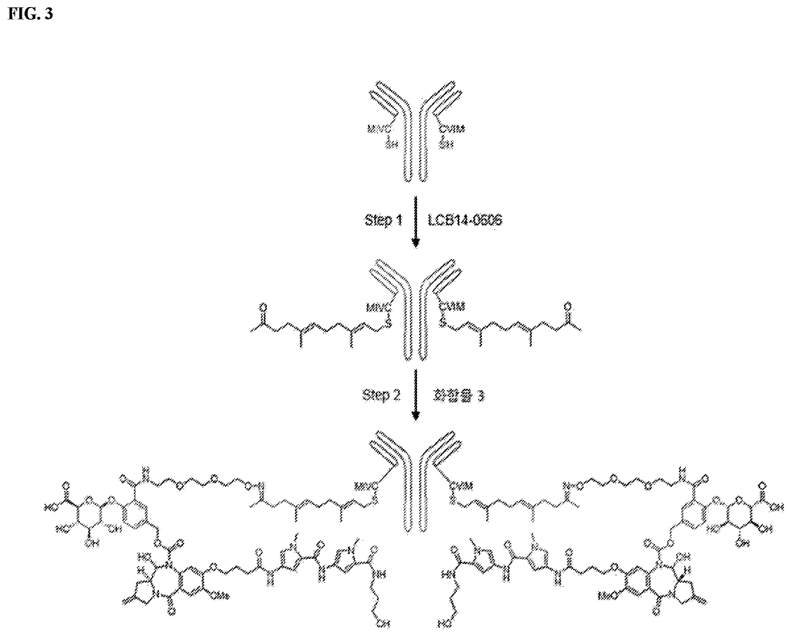

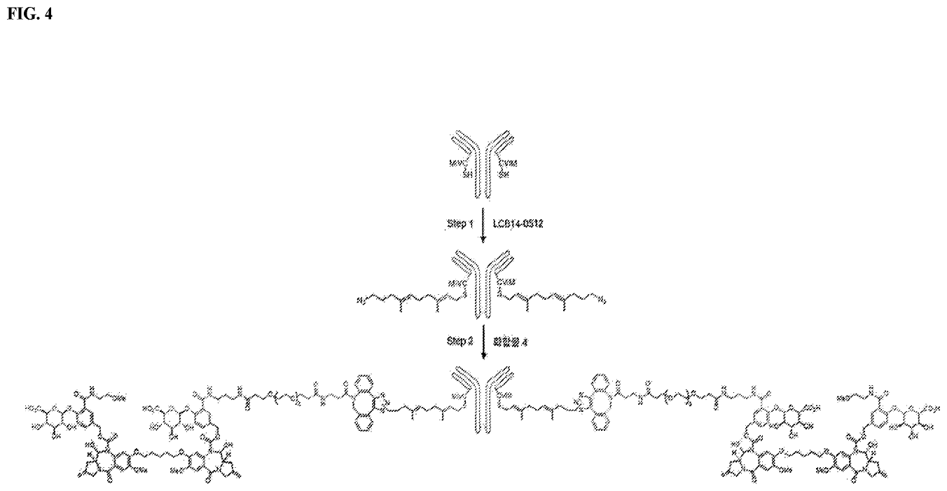

ADCs produced through the above two steps are shown in Table 4 and FIGS. 1 to 4 below.

TABLE 4

| ADC | Antibody | Prenylation group | toxin |
|---|---|---|---|
| ADC1 | 2G10-5-CaaX | LCB14-0606 | Compound 1 |
| ADC2 | | | Compound 2 |
| ADC3 | | | Compound 3 |
| ADC4 | | LCB14-0512 | Compound 4 |

### <Experimental Example 1> In Vitro Cytotoxicity Evaluation

The cell proliferation inhibitory activity of the ADC produced according to <Example 2> and the drug alone included in the ADC against cancer cell lines were measured.

Specifically, human pancreatic cancer cell lines (BxPC-3, Capan-1, and PATU-8988s), breast cancer cell lines (JIMT-1 and MDA-MB-468), gastric cancer cell lines (NCI-N87, SNU-601, and SNU-620), colon cancer cell lines (HCT15, HT29, DLD-1, SW480, and COLO205), an ovarian cancer cell line (OVCAR-3), prostate cancer cell lines (PC-3 and LNCaP), and non-small cell lung cancer cell lines (NCI-H1781, HCC827, Calu-1, and Calu-3), which are commercially available as cancer cell lines, were used. MMAE as a drug alone included in the ADC prepared according to Example 3 and SG2057 (CAS #: 260417-62-7, MedKoo Biosciences) as a PBD dimer were used. 500 cells/well to 5,000 cells/well of each cancer cell line were seeded into a 96-well plate and cultured for 24 hours, and then each of ADC1, ADC2 and ADC3 produced according to Example 3 and the drugs MMAE and SG2057 alone were treated at a concentration of 0.256 pM to 100 nM (5-fold serial dilution). After 144 hours, the number of living cells was quantified using a sulforhodamine B (SRB) dye or Cell titer glo.

As a result, as shown in Tables 5 to 7, it was confirmed that pyrrolobenzodiazepine-based antibody-drug conjugates (ADC2 and ADC3) exhibited much stronger cytotoxicity in most of the cancer cell lines, compared to an auristatin-based antibody-drug conjugate (ADC1).

TABLE 5

| Test | CC₅₀ (nM) | | | | | | | |
|---|---|---|---|---|---|---|---|---|
| samples | BxPC-3 | Capan-1 | Patu8988S | JIMT-1 | MDA-MB-468 | NCI-N87 | SNU-601 | SNU-620 |
| MMAE | 0.606 | 0.520 | 0.334 | 0.154 | 0.640 | 0.725 | 0.268 | 0.651 |
| SG2057 | — | — | — | — | — | — | — | — |
| ADC1 | 4.639 | 18.060 | 1.412 | 0.231 | 6.104 | 0.582 | 7.267 | 19.180 |
| ADC2 | 3.605 | — | — | — | 0.580 | 0.148 | — | — |
| ADC3 | 53.680 | — | — | — | 11.540 | 0.357 | — | — |

TABLE 6

| Test | CC₅₀ (nM) | | | | | | | |
|---|---|---|---|---|---|---|---|---|
| samples | HCT15 | HT29 | DLD-1 | SW480 | COLO205 | OVCAR-3 | PC-3 | LNCaP |
| MMAE | 3.865 | 0.330 | 8.883 | 3.825 | 0.628 | 0.304 | 0.946 | 0.551 |
| SG2057 | 0.161 | 0.020 | 0.050 | — | — | — | — | — |
| ADC1 | >100 | 4.043 | >100 | >100 | 11.950 | 10.030 | 42.900 | 44.450 |
| ADC2 | 57.160 | 1.065 | 31.030 | — | — | — | — | — |
| ADC3 | 42.000 | 5.435 | 42.470 | — | — | — | — | — |

TABLE 7

CC₅₀ (nM)

| Test samples | NCI-H1781 | HCC827 | Calu-1 | Calu-3 |
|---|---|---|---|---|
| MMAE | 0.208 | 0.674 | 0.971 | 0.400 |
| SG2057 | — | — | — | — |
| ADC1 | 3.220 | 11.290 | 59.580 | 3.538 |
| ADC2 | — | — | — | — |
| ADC3 | — | — | — | — |

### <Experimental Example 2> Analysis of In Vivo Tumor Growth Inhibitory Efficacy

In a tumor-transplanted mouse model, the tumor growth inhibitory efficacy of the ADCs produced according to Example 3 was analyzed.

Specifically, pancreatic cancer cell lines (BxPC-3 and Capan-1), a breast cancer cell line (MDA-MB-468), a gastric cancer cell line (NCI-N87), a colon cancer cell line (HT29), and non-small cell lung cancer cell lines (HCC827 and NCI-H2170) were cultured. The cultured cells were suspended in 100 μl of PBS so that the number of cells became 1×10⁶ to 7×10⁶, mixed with 100 μl of Matrigel, and then engrafted into Balb/c-nude mice. When the size of a tumor reached 100 mm³ to 200 mm³, each of the ADCs produced according to Example 3 was intravenously administered according to the dosage regimen shown in Tables 8 to 10 below. The size of the tumor transplanted in mice was measured immediately before the initial administration (Day 1), and then periodically measured for 56 days. In the case of a control, Trodelvy (sacituzumab govitecan-hziy) or DS-1062BS (Datopotamab deruxtecan) was administered.

TABLE 8

| | | |
|---|---|---|
| Primary | | |
| (QD*1) | MDA-MB-468 | BxPC-3 |

| | | | |
|---|---|---|---|
| ADC1 | 2, 4 | mpk | 2, 4 mpk |
| ADC2 | 0.2, 0.3, 0.4 | mpk | — |
| ADC3 | 1, 4 | mpk | — |

TABLE 9

| Secondary | MDA-MB-468 | BxPC-3 | NCI-N87 | HCC827 |
|---|---|---|---|---|
| ADC1 | 0.5 mpk | 0.5 mpk | 2 mpk | 4 mpk |
| | (Q4D*4) | (2QW*4) | (QD*1) | (2QW*2) |
| | 1 mpk | 1 mpk | 4 mpk | 4 mpk |
| | (Q4D*4) | (2QW*2) | (QD*1) | (QD*1) |
| | 2 mpk | 2 mpk | | 10 mpk |
| | (QD*1) | (QD*1) | | (QD*1) |
| Trodelvy | 12.5 mpk | 25 mpk | 17.5 mpk | 12.5 mpk |
| | (Q4D*4) | (2QW*4) | (2QW*4) | (2QW*2) |

TABLE 10

| Tertiary | BxPC-3 | HCC827 | Capan-1 | NCI-H2170 | HT-29 |
|---|---|---|---|---|---|
| ADC1 | 2 mpk | 4 mpk | 10 mpk | 4 mpk | 5 mpk |
| | (QD*1) | (2QW*2) | (QD*1) | (2QW*2) | (QD*1) |
| | 2 mpk | 10 mpk | 10 mpk | 10 mpk | 5 mpk |
| | (2QW*2) | (QD*1) | (QW*3) | (QD*1) | (2QW*2) |
| Trodelvy | 25 mpk | 12.5 mpk | 25 mpk | 25 mpk | 12.5 mpk |
| | (2QW*4) | (2QW*4) | (2QW*4) | (2QW*4) | (2QW*4) |
| DS- | 3 mpk | 10 mpk | 10 mpk | 4 mpk | 5 mpk |
| 1062BS | (QW*2) | (QD*1) | (QD*1) | (2QW*2) | (2QW*2) |
| | | | 10 mpk | 10 mpk | |
| | | | (QW*3) | (QD*1) | |

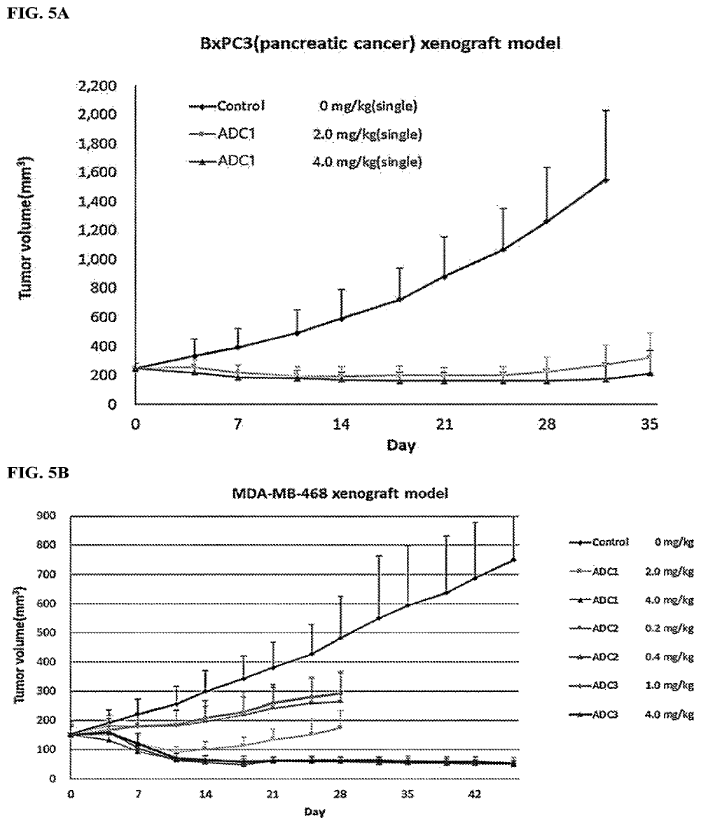

As a result, as illustrated in FIGS. 5A and 5B, it was confirmed through a first experiment that, when an auristatin-based antibody-drug conjugate (ADC1) using an oxime reaction was administered to a pancreatic cancer cell line-transplanted mouse model, both the cases of 2 mg/kg and 4 mg/kg exhibited tumor growth inhibitory efficacy in a dose-dependent manner compared to the control (FIG. 5A). In the breast cancer cell line-transplanted mouse model, tumor growth inhibitory efficacy was confirmed in both the group administered with the auristatin-based antibody-drug conjugate (ADC1) using an oxime reaction and the groups administered with the pyrrolobenzodiazepine-based antibody-drug conjugates (ADC2 and ADC3).

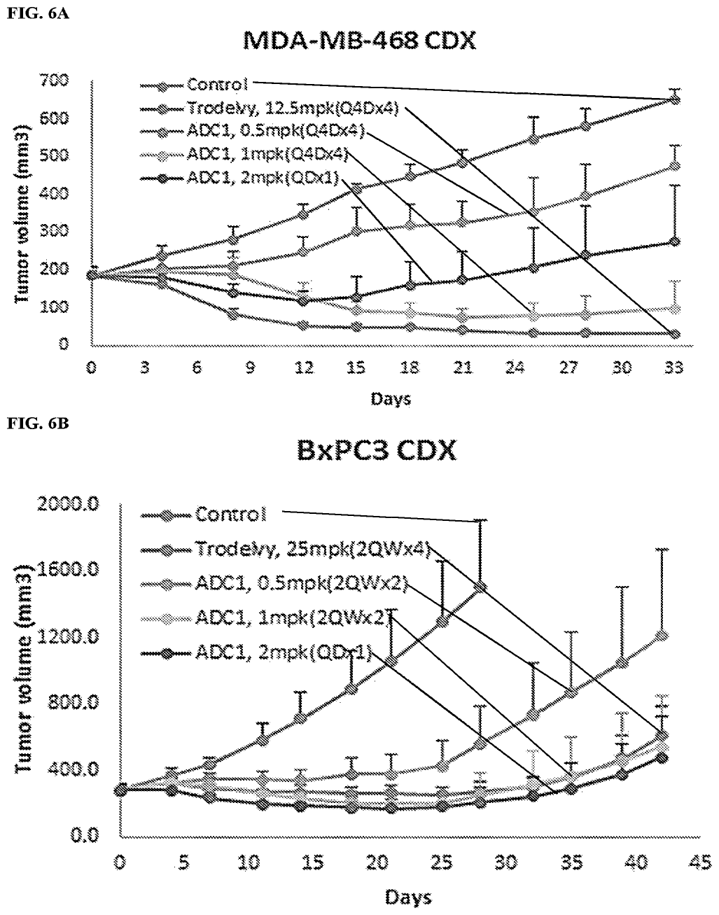

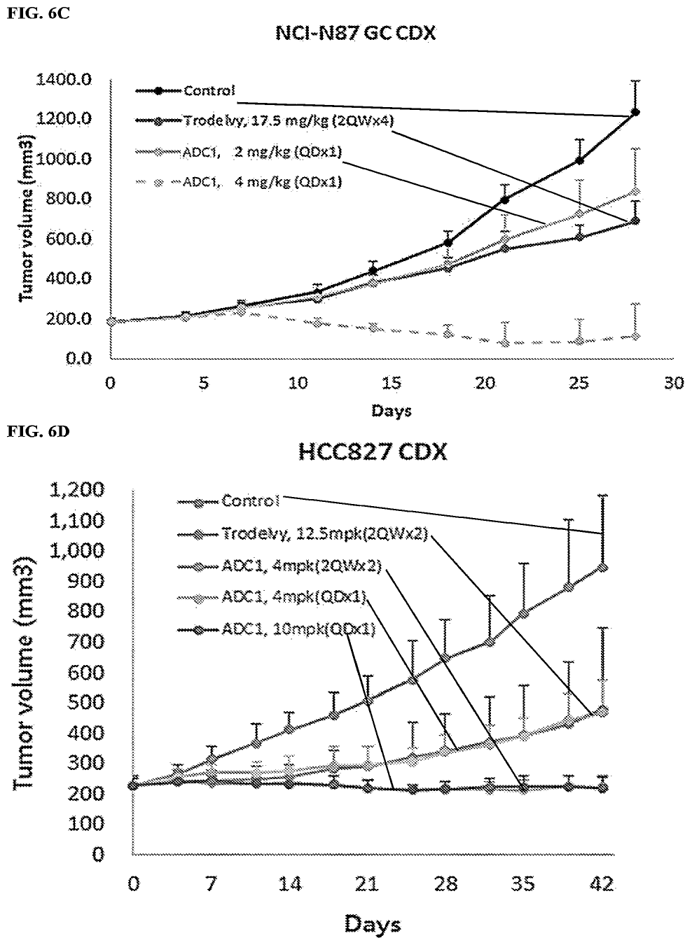

As illustrated in FIGS. 6A to 6D, it was also confirmed through a second experiment that ADC1 had excellent efficacy compared to Trodelvy, which is a commercially available drug, in all of the four cancer cell line-transplanted mouse models (MDA-MB-468, BxPC-3, NCI-N87, and HCC827).

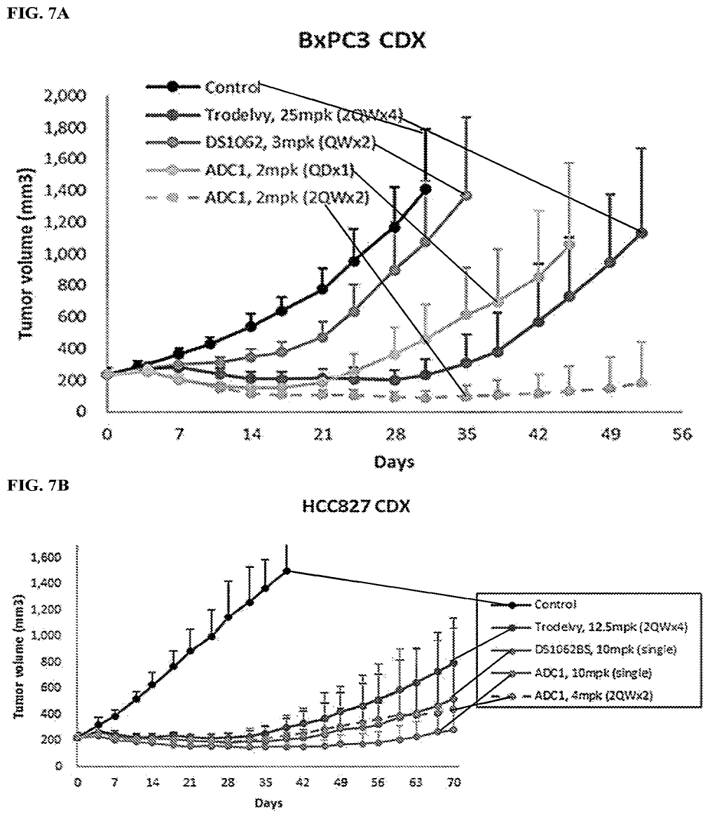

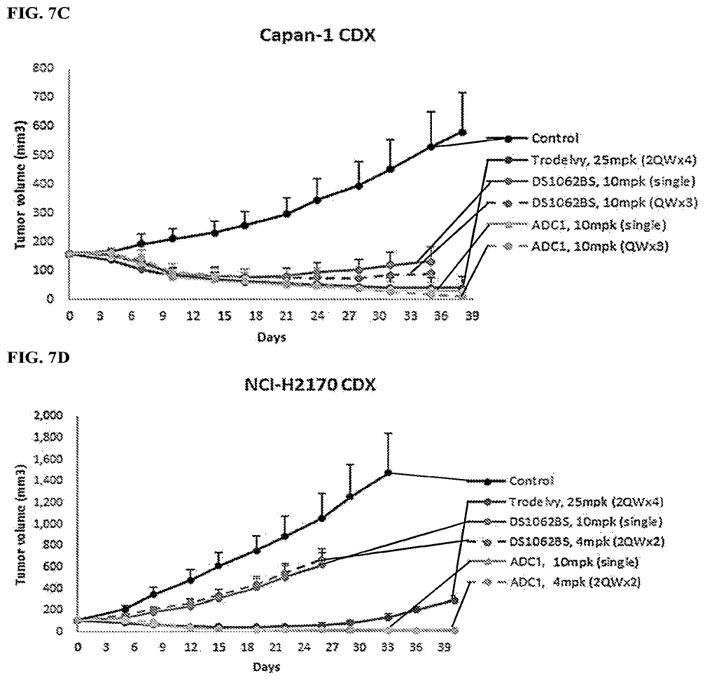

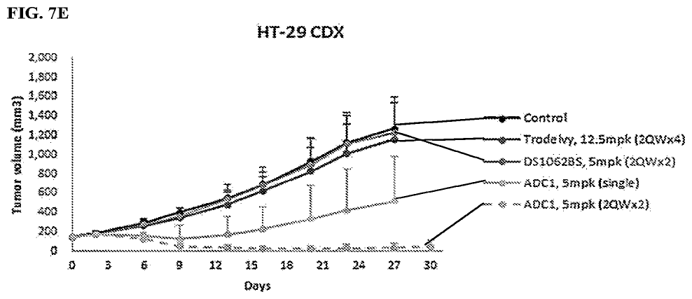

As illustrated in FIGS. 7A to 7E, it was also confirmed through a third experiment that ADC1 had excellent efficacy compared to Trodelvy, which is a commercially available drug, in all of the five cancer cell line-transplanted mouse models (BxPC-3, HCC827, Capan-1, NCI-H2170, and HT-29), and ADC1 had efficacy equivalent to DS-1062BS, which is a competitive drug.

We claim:

1. A conjugate having a structure represented by General Formula I or a pharmaceutically acceptable salt thereof:
Ab-[L-(B)₁]ₘ [General Formula I]
wherein,
Ab is an anti-TROP2 (tumor-associated calcium signal transducer 2 aka TACSTD2) antibody or antigen-binding fragment thereof comprising a heavy chain variable region and a light chain variable region, wherein
the heavy chain variable region comprises a heavy chain CDR1 comprising the amino acid sequence of SEQ ID NO: 2, a heavy chain CDR2 comprising the amino acid sequence of SEQ ID NO: 4, and a heavy chain CDR3 comprising the amino acid sequence of SEQ ID NO: 6; and
the light chain variable region comprises a light chain CDR1 comprising the amino acid sequence of SEQ ID NO: 9, a light chain CDR2 comprising the amino acid sequence of SEQ ID NO: 11, and a light chain CDR3 comprising the amino acid sequence of SEQ ID NO: 13;
L is a linker;
B is an active agent; and
1 and m are each independently 1 to 20.

2. The conjugate of claim 1, wherein the antibody or antigen-binding fragment thereof comprises a heavy chain variable region comprising:
the amino acid sequence of SEQ ID NO: 15 or 20;
a sequence having at least 90% sequence identity to the amino acid sequence of SEQ ID NO: 15 or 20 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6; or
a sequence having at least 95% sequence identity to the amino acid sequence of SEQ ID NO: 15 or 20 while maintaining the heavy chain CDR1 comprising the amino acid sequence of SEQ ID NO: 2, the heavy chain CDR2 comprising the amino acid sequence of SEQ ID NO: 4, and the heavy chain CDR3 comprising the amino acid sequence of SEQ ID NO: 6.

3. The conjugate of claim 1 or 2, wherein the antibody or antigen-binding fragment thereof comprises a light chain variable region comprising:
the amino acid sequence of SEQ ID NO: 16;
a sequence having at least 90% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13; or
a sequence having at least 95% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13.

4. The conjugate of claim 1, wherein the antibody or antigen-binding fragment thereof comprises a heavy chain variable region comprising the amino acid sequence of SEQ ID NO: 15 and a light chain variable region comprising the amino acid sequences of SEQ ID NO: 16.

5. The conjugate of any one of claims 1-4, wherein the antibody is a humanized antibody.

6. The conjugate of any one of claims 1-5, wherein the humanized antibody comprises:
(i) a variable heavy chain framework region from a heavy chain of a human antibody or from a human consensus framework, wherein the variable heavy chain framework region comprises one or more of the following amino acid sequence changes: Y27F, T30S, V37L, M48I, G49A, 170L, and R72V; and
(ii) a variable light chain framework region from a light chain of a human antibody or from a human consensus framework, wherein the variable light chain framework region comprises the following amino acid sequence change: Y49S.

7. The conjugate of any one of claims 1-6, wherein the antibody or antigen-binding fragment thereof is selected from a monoclonal antibody, a domain antibody (dAb), a single chain antibody (scAb), a Fab fragment, a Fab′ fragment, a F(ab′)2 fragment, an scFab fragment, an Fv fragment, a dsFv fragment, a single chain variable fragment (scFv), an scFv-Fc fragment, a single domain heavy chain antibody, a single domain light chain antibody, a variant antibody, a multimeric antibody, a minibody, a diabody, a bispecific antibody, and a multispecific antibody.

8. The conjugate of any one of claims 1-7, wherein L is cleavable.

9. The conjugate of any one of claims 1-8, wherein the conjugate has a structure represented by General Formula IIa or a pharmaceutically acceptable salt thereof: 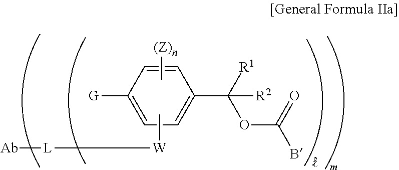
wherein
each B′ is an active agent;
each G is independently a glucuronic acid moiety or 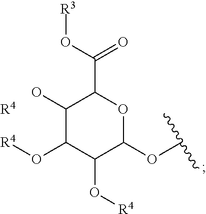
R³ is hydrogen or a carboxyl-protecting group;
each R⁴ is independently hydrogen or a hydroxyl-protecting group;
R¹ and R² are each independently hydrogen, C₁₋₈ alkyl, or C₃₋₈ cycloalkyl;
W is —C(O)—, —C(O)NR′—, —C(O)O—, —SO₂NR′—, —P(O)R″NR′, —SONR′—, or —PO₂NR′—, wherein the C, S, or P is directly bonded to the phenyl ring of Formula IIa;
R′ and R″ are each independently hydrogen, C₁₋₈ alkyl, C₃₋₈ cycloalkyl, C₁₋₈ alkoxy, C₁₋₈ alkylthio, mono- or di-C₁₋₈ alkylamino, C₃₋₂₀ heteroaryl, or C₆₋₂₀ aryl;
each Z is independently hydrogen, C₁₋₈ alkyl, halogen, cyano, or nitro;
n is 0, 1, 2, or 3;
L comprises:
A) C₁₋₅₀ alkylene or C₁₋₅₀ heteroalkylene and comprises
(i) one or more unsaturated bonds
(ii) a heteroarylene; or
(iii) is substituted with at least one C₁₋₂₀ alkyl; or
B) at least one isoprenyl group having a structure represented by General Formula III: 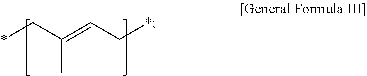
and
1 and m are each independently 1 to 20.

10. A conjugate having a structure represented by General Formula IIa or a pharmaceutically acceptable salt thereof: 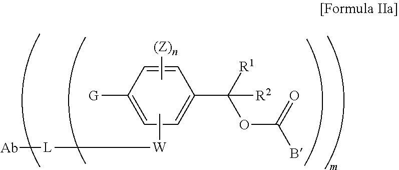
wherein
Ab is an anti-TROP2 (tumor-associated calcium signal transducer 2 aka TACSTD2) antibody or antigen-binding fragment thereof;
each B′ is an active agent;
each G is independently a glucuronic acid moiety or 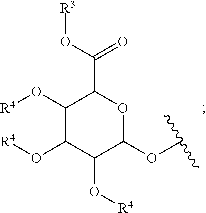
R³ is hydrogen or a carboxyl-protecting group;
each R⁴ is independently hydrogen or a hydroxyl-protecting group;
R¹ and R² are each independently hydrogen, C₁₋₈ alkyl, or C₃₋₈ cycloalkyl;
W is —C(O)—, —C(O)NR′—, —C(O)O—, —SO₂NR′—, —P(O)R″NR′, —SONR′—, or —PO₂NR′—, wherein the C, S, or P is directly bonded to the phenyl ring of Formula IIa;
R′ and R″ are each independently hydrogen, C₁₋₈ alkyl, C₃₋₈ cycloalkyl, C₁₋₈ alkoxy, C₁₋₈ alkylthio, mono- or di-C₁₋₈ alkylamino, C₃₋₂₀ heteroaryl, or C₆₋₂₀ aryl;
each Z is independently hydrogen, C₁₋₈ alkyl, halogen, cyano, or nitro;
n is 0, 1, 2, or 3;
L comprises:
A) C₁₋₅₀ alkylene or C₁₋₅₀ heteroalkylene and comprises
(i) one or more unsaturated bonds
(ii) a heteroarylene; or
(iii) is substituted with at least one C₁₋₂₀ alkyl; or
B) at least one isoprenyl group having a structure represented by General Formula III: 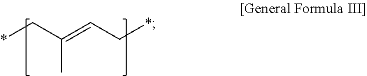
and
1 and m are each independently 1 to 20.

11. The conjugate of claim 10, wherein the antibody or antigen-binding fragment thereof comprises a heavy chain variable region and a light chain variable region, wherein:
the heavy chain variable region comprises a heavy chain CDR1 comprising the amino acid sequence of SEQ ID NO: 2, a heavy chain CDR2 comprising the amino acid sequence of SEQ ID NO: 4, a and heavy chain CDR3 comprising the amino acid sequence of SEQ ID NO: 6; and
the light chain variable region comprises a light chain CDR1 comprising the amino acid sequence of SEQ ID NO: 9, a light chain CDR2 comprising the amino acid sequence of SEQ ID NO: 11, and a light chain CDR3 comprising the amino acid sequence of SEQ ID NO: 13.

12. The conjugate of claim 11, wherein the antibody or antigen-binding fragment thereof comprises a heavy chain variable region comprising:
the amino acid sequence of SEQ ID NO: 15 or 20;
a sequence having at least 90% sequence identity to the amino acid sequence of SEQ ID NO: 15 or 20 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13; or
a sequence having at least 95% sequence identity to the amino acid sequence of SEQ ID NO: 15 or 20 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13.

13. The conjugate of claim 11 or 12, wherein the antibody or antigen-binding fragment thereof comprises a light chain variable region comprising:
the amino acid sequence of SEQ ID NO: 16;
a sequence having at least 90% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13; or
a sequence having at least 95% sequence identity to the amino acid sequence of SEQ ID NO: 16 while maintaining the light chain CDR1 comprising the amino acid sequence of SEQ ID NO: 9, the light chain CDR2 comprising the amino acid sequence of SEQ ID NO: 11, and the light chain CDR3 comprising the amino acid sequence of SEQ ID NO: 13.

14. The conjugate of claim 11, wherein the antibody or antigen-binding fragment thereof comprises a heavy chain variable region comprising the amino acid sequence of SEQ ID NO: 15 and a light chain variable region comprising the amino acid sequences of SEQ ID NO: 16.

15. The conjugate of any one of claims 11-14, wherein the antibody is a humanized antibody.

16. The conjugate of any one of claims 11-15, wherein the humanized antibody comprises:
(i) a variable heavy chain framework region from a heavy chain of a human antibody or from a human consensus framework, wherein the variable heavy chain framework region comprises one or more of the following amino acid sequence changes: Y27F, T30S, V37L, M48I, G49A, 170L, and R72V; and
(ii) a variable light chain framework region from a light chain of a human antibody or from a human consensus framework, wherein the variable light chain framework region comprises the following amino acid sequence change: Y49S.

17. The conjugate of any one of claims 11-16, wherein the antibody or antigen-binding fragment thereof is selected from a monoclonal antibody, a domain antibody (dAb), a single chain antibody (scAb), a Fab fragment, a Fab′ fragment, a F(ab′)2 fragment, an scFab fragment, an Fv fragment, a dsFv fragment, a single chain variable fragment (scFv), an scFv-Fc fragment, a single domain heavy chain antibody, a single domain light chain antibody, a variant antibody, a multimeric antibody, a minibody, a diabody, a bispecific antibody, and a multispecific antibody.

18. The conjugate of any one of claims 9-17, wherein each G is

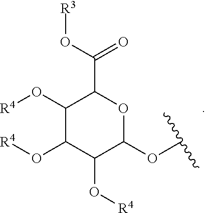

19. The conjugate of any one of claims 9-18, wherein R¹ and R² are each hydrogen.

20. The conjugate of any one of claims 9-19, wherein R³ is hydrogen.

21. The conjugate of any one of claims 9-20, wherein each R⁴ is a hydroxyl-protecting group.

22. The conjugate of any one of claims 9-21, wherein n is 0.

23. The conjugate of any one of claims 9-22, wherein each W is —C(O)NR′—, further wherein the C is directly bonded to the phenyl ring of Formula IIa, and NR′ is bonded to L.

24. The conjugate of any one of claims 9-23, wherein each R⁴ is independently hydrogen.

25. The conjugate of any one of claims 9-18, wherein:
R¹ and R² are each hydrogen;
n is 0; and
each W is —C(O)NR′—, C is directly bonded to the phenyl ring of Formula IIa, and R′ is hydrogen, C₁₋₈ alkyl, C₃₋₈ cycloalkyl, C₁₋₈ alkoxy, C₁₋₈ alkylthio, mono- or di-C₁₋₈ alkylamino, C₃₋₂₀ heteroaryl, or C₆₋₂₀ aryl, wherein NR′ is bonded to L.

26. The conjugate of any one of claims 9-25, wherein L comprises a nitrogen-containing 1- to 50-membered heteroalkylene.

27. The conjugate of any one of claims 9-26, wherein L comprises a hydrophilic amino acid.

28. The conjugate of claim 27, wherein W comprises two or more atoms of the hydrophilic amino acid, and the nitrogen of W forms a peptide bond with a carbonyl of the hydrophilic amino acid.

29. The conjugate of any one of claims 9-28, wherein
L is a nitrogen-containing 1- to 50-membered heteroalkylene,
the linker comprises two or more atoms of a hydrophilic amino acid, and
the nitrogen forms a peptide bond with a carbonyl of the hydrophilic amino acid.

30. The conjugate of any one of claims 9-29, wherein L is covalently bonded to the antibody by a thioether bond and the thioether bond comprises a sulfur atom of a cysteine of the antibody.

31. The conjugate of claim 30, wherein:
the antibody comprises an amino acid motif recognizable by an isoprenoid transferase at the C-terminus of the antibody, and
the thioether bond comprises a sulfur atom of a cysteine of the amino acid motif.

32. The conjugate of claim 31, wherein the amino acid motif has a CYYX sequence, further wherein:
C is cysteine;
Y is an aliphatic amino acid;
X is selected from glutamine, glutamate, serine, cysteine, methionine, alanine, and leucine; and
the thioether bond comprises a sulfur atom of a cysteine of the amino acid motif.

33. The conjugate of claim 31, wherein the amino acid motif has a CYYX sequence further wherein:
Y is selected from alanine, isoleucine, leucine, methionine, and valine.

34. The conjugate of claim 31, wherein the amino acid motif has a CVIM (SEQ ID NO: 24) or CVLL sequence (SEQ ID NO: 25).

35. The conjugate of any one of claims 31-34, wherein at least one of 1 to 20 amino acids preceding the amino acid motif is glycine.

36. The conjugate of any one of claims 1-35, wherein L comprises the amino acid sequence of GGGGGGGCVIM (SEQ ID NO: 22) at the C-terminus.

37. The conjugate of any one of claims 1-36, wherein L comprises a C₁₋₅₀ heteroalkylene.

38. The conjugate of any one of claims 1-37, wherein L comprises an oxime.

39. The conjugate of claim 38, wherein the oxygen atom of the oxime is on the side of L linked to W and the carbon atom of the oxime is on the side of L linked to Ab.

40. The conjugate of claim 38, wherein the carbon atom of the oxime is on the side of L linked to W and the oxygen atom of the oxime is on the side of L linked to Ab.

41. The conjugate of any one of claims 1-36, wherein
L is a C₁₋₅₀ heteroalkylene containing an oxime,
the oxygen atom of the oxime is on the side of L linked to W,
the carbon atom of the oxime is on the side of L linked to Ab, or
the carbon atom of the oxime is on the side of L linked to W, and
the oxygen atom of the oxime is on the side of L linked to Ab.

42. The conjugate of any one of claims 1-41, wherein L comprises an oxime, and at least one isoprenyl unit covalently bonds the oxime to Ab (e.g., at least one isoprenyl unit directly or indirectly bonds the oxime to Ab).

43. The conjugate of any one of claims 1-42, wherein L further comprises a connecting unit represented by General Formula VIII or General Formula IX:
—(CH₂)ᵣ(V(CH₂)ₚ)_{q}— [General Formula VIII]
—(CH₂CH₂X)_{w}— [Formula IX]
V is a single bond, —O—, —S—, —NR²¹—, —C(O)NR²²—, —NR²³C(O)—, —NR²⁴SO₂—, or —SO₂NR²⁵—;
X is —O—, C₁₋₈ alkylene, or —NR²¹—;
R²¹ to R²⁵ are each independently hydrogen, C₁₋₆ alkyl, C₁₋₆ alkyl C₆₋₂₀ aryl, or C₁₋₆ alkyl-C₃₋₂₀ heteroaryl;
r is 0 to 10;
p is 0 to 10;
q is 1 to 20; and
w is 1 to 20.

44. The conjugate of claim 43, wherein q is 1 to 10.

45. The conjugate of claim 43 or 44, wherein r is 1 or 2.

46. The conjugate of any one of claims 43-45, wherein p is 1 or 2.

47. The conjugate of any one of claims 43-46, wherein V is —O—.

48. The conjugate of claim 43, wherein:
q is 1 to 10;
r and p are each 1 or 2; and
V is —O—.

49. The conjugate of any one of claims 43-48, wherein X is —O—.

50. The conjugate of any one of claims 43-48, wherein w is 1 to 10.

51. The conjugate of any one of claims 43-48, wherein:
the X is —O—; and
w is 1 to 10.

52. The conjugate of any one of claims 43-51, wherein L comprises 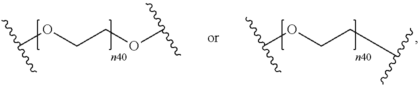
wherein n40 is 1-10, preferably at least 2.

53. The conjugate of any one of claims 43-52, wherein L comprises an oxime, and at least one polyethylene glycol unit covalently bonds the oxime to an active agent.

54. The conjugate of any one of claims 43-53, wherein L comprises a binding unit formed by a reaction between an alkyne and an azide or between an aldehyde or ketone group and hydrazine or hydroxylamine.

55. The conjugate of any one of claims 43-54, wherein L further comprises a binding unit represented by General Formula IVa, IVb, IVc, IVd, or IVe below: 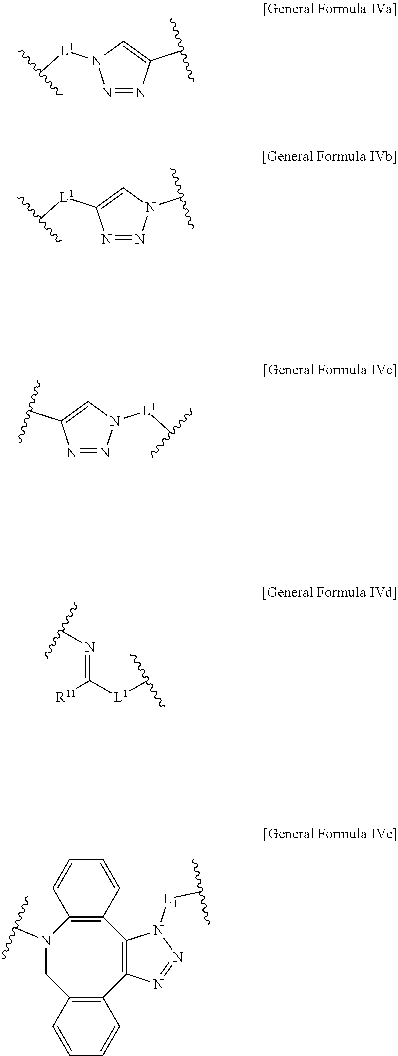
wherein
L₁ and L₂ are each independently a single bond or C₁₋₃₀ alkylene; and
R₁₁ is hydrogen or C₁₋₁₀ alkyl.

56. The conjugate of claim 55, wherein L₁ and L₂ are each independently a single bond, C₁₁ alkylene, or C₁₂ alkylene.

57. The conjugate of any one of claims 3-56, wherein the isoprenoid transferase is farnesyl protein transferase (FTase) or geranylgeranyl transferase (GGTase).

58. The conjugate of any one of claims 1-57, wherein L is branched and comprises:
i) a branching unit covalently coupled to the antibody by a primary linker;
ii) a first branch, in which a first active agent is covalently coupled to the branching unit by a secondary linker and a cleavage group; and
iii) a second branch, in which:
a) a second active agent is covalently coupled to the branching unit by a secondary linker and a cleavage group; or
b) a polyethylene glycol moiety is covalently coupled to the branching unit.

59. The conjugate of claim 58, wherein the branching unit has a structure represented by 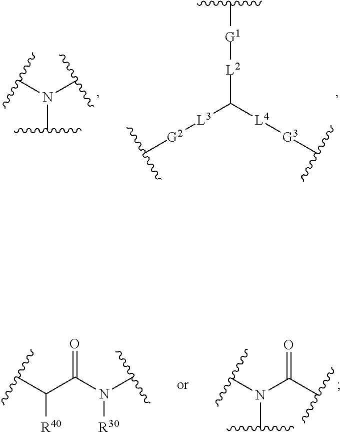
wherein
L², L³, and L⁴ are each independently a bond or —CₙH₂ₙ—,
n is 1 to 30;
G¹, G², and G³ each independently represent a bond, 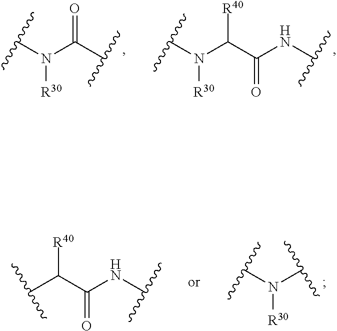
R³⁰ is hydrogen or C₁₋₃₀ alkyl;
R⁴⁰ is hydrogen or L₅-COOR₆; and
L₅ is a bond or —C_{n′}H_{2n′}—;
n′ is 1 to 10; and
R₆ is hydrogen or C₁₋₃₀ alkyl.

60. The conjugate of claim 58 or 59, wherein the cleavage group is cleavable in a target cell and is capable of releasing one or more active agents.

61. The conjugate of any one of claims 58-60, wherein:
at least one branched linker is covalently coupled to Ab; and
at least two active agents are covalently coupled to the branched linker.

62. The conjugate of claim 61, wherein one branched linker is coupled to Ab.

63. The conjugate of claim 61, wherein two branched linkers are coupled to Ab.

64. The conjugate of claim 61, wherein three branched linkers are coupled to Ab.

65. The conjugate of claim 61, wherein four branched linkers are coupled to Ab.

66. The conjugate of any one of claims 59-65, wherein each branched linker is coupled to two active agents.

67. The conjugate of any one of claims 59-66, wherein the conjugate comprises at least two different active agents.

68. The conjugate of any one of claims 59-67, wherein at least one branched linker is coupled to two different active agents.

69. The conjugate of any one of claims 59-68, wherein the branching unit is a nitrogen atom.

70. The conjugate of any one of claims 59-68, wherein the branching unit is an amide and the primary linker comprises the carbonyl of the amide.

71. The conjugate of any one of claims 59-69, wherein the branching unit is an amide and the secondary linker comprises the carbonyl of the amide.

72. The conjugate of any one of claims 59-71, wherein the branching unit is lysine.

73. The conjugate of any one of claims 1-72, wherein the conjugate comprises a structure represented by: 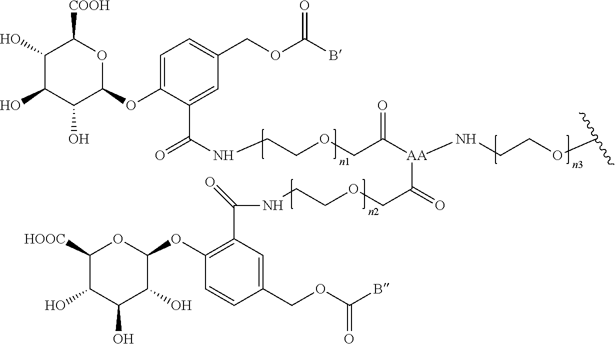
or a pharmaceutically acceptable salt thereof; wherein
B′ and B″ are each active agents;
n1 to n3 are each independently 0 to 30; and
AA is an amino acid group.

74. The conjugate of any one of claims 1-73, wherein the conjugate comprises a structure represented by: 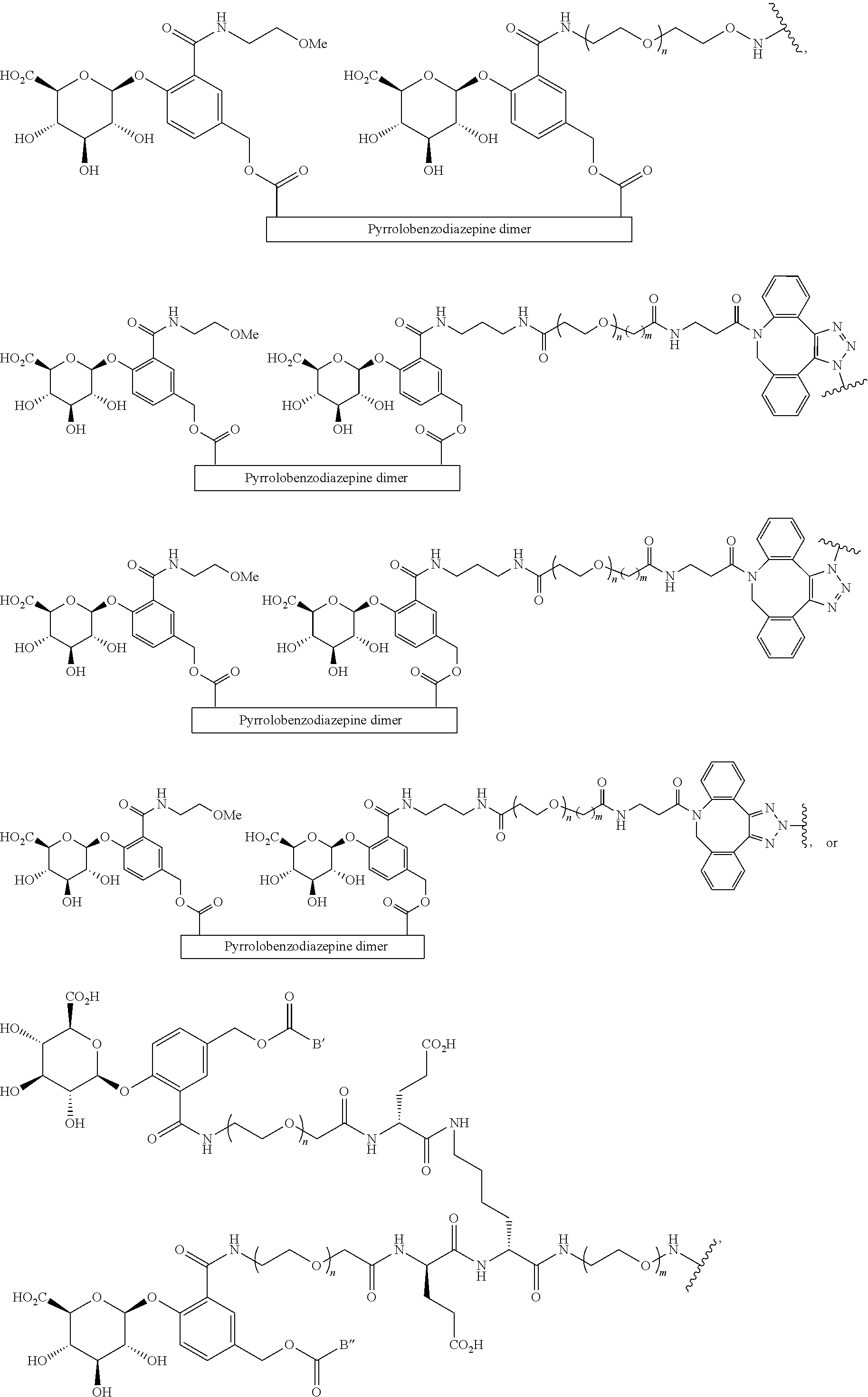
or a pharmaceutically acceptable salt thereof;
wherein B′ and B″ refer to active agents which are identical to or different from each other;
the pyrrolobenzodiazepine dimer, for example, a compound of formula III; and
m and n each independently refers to 0 to 30,
or a pharmaceutically acceptable salt thereof.

75. The conjugate of any one of claims 1-74, wherein the active agent is a chemotherapeutic agent or a toxin.

76. The conjugate of any one of claims 1-75, wherein the active agent is an immunomodulatory compound, an anticancer agent, an anti-viral agent, an anti-bacterial agent, an anti-fungal agent, an anti-parasitic agent, or a combination thereof.

77. The conjugate of any one of claims 1-73, wherein the active agent is selected from:
(a) erlotinib, bortezomib, fulvestrant, sutent, letrozole, imatinib mesylate, PTK787/ZK 222584, oxaliplatin, 5-fluorouracil, leucovorin, rapamycin, lapatinib, lonafarnib, sorafenib, gefitinib, AG1478, AG1571, thiotepa, cyclophosphamide, busulfan, improsulfan, piposulfan, benzodopa, carboquone, meturedopa, uredopa, ethylenimine, altretamine, triethylenemelamine, triethylenephosphoramide, triethiylenethiophosphoramide, trimethylolomelamine, bullatacin, bullatacinone, camptothecin, topotecan, bryostatin, callystatin, CC-1065, adozelesin, carzelesin, bizelesin, cryptophycin 1, cryptophycin 8, dolastatin, duocarmycin, KW-2189, CB1-TM1, eleutherobin, pancratistatin, sarcodictyin, spongistatin, chlorambucil, chlornaphazine, cholophosphamide, estramustine, ifosfamide, mechlorethamine, melphalan, novembichin, phenesterine, prednimustine, trofosfamide, uracil mustard, carmustine, chlorozotocin, fotemustine, lomustine, nimustine, ranimnustine, calicheamicin, calicheamicin gamma 1, calicheamicin omega 1, dynemicin, dynemicin A, clodronate, esperamicin, neocarzinostatin chromophore, aclacinomysins, actinomycin, antrmycin, azaserine, bleomycins, cactinomycin, carabicin, carninomycin, carzinophilin, chromomycins, dactinomycin, daunorubicin, detorubucin, 6-diazo-5-oxo-L-norleucine, doxorubicin, morpholino-doxorubicin, cyanomorpholino-doxorubicin, 2-pyrrolino-doxorubicin, liposomal doxorubicin, deoxydoxorubicin, epirubicin, esorubicin, marcellomycin, mitomycin C, mycophenolic acid, nogalamycin, olivomycins, peplomycin, potfiromycin, puromycin, quelamycin, rodorubicin, streptomigrin, streptozocin, tubercidin, ubenimex, zinostatin, zorubicin, 5-fluorouracil, denopterin, methotrexate, pteropterin, trimetrexate, fludarabine, 6-mercaptopurine, thiamiprine, thiguanine, ancitabine, azacitidine, 6-azauridine, carmofur, cytarabine, dideoxyuridine, doxifluridine, enocitabine, floxuridine, calusterone, dromostanolone, propionate, epitiostanol, mepitiostane, testolactone, aminoglutethimide, mitotane, trilostane, folinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, 2-ethylhydrazide, procarbazine, polysaccharide-k, razoxane, rhizoxin, sizofiran, spirogermanium, tenuazonic acid, triaziquone, 2,2′,2″-trichlorotriethylamine, T-2 toxin, verracurin A, roridin A, anguidine, urethane, vindesine, dacarbazine, mannomustine, mitobronitol, mitolactol, pipobroman, gacytosine, arabinoside, cyclophosphamide, thiotepa, paclitaxel, paclitaxel, albumin-engineered nanoparticle formulation of paclitaxel, docetaxel, chlorambucil, gemcitabine, 6-thioguanine, mercaptopurine, cisplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, CPT-11, topoisomerase inhibitor RFS 2000, difluoromethylornithine, retinoic acid, capecitabine, or a pharmaceutically acceptable salt, solvate or acid thereof;
(b) monokines, lymphokines, traditional polypeptide hormones, parathyroid hormones, thyroxine, relaxin, prorelaxin, glycoprotein hormone, follicle stimulating hormone, thyroid stimulating hormone, luteinizing hormone, hepatic growth factor fibroblast growth factor, prolactin, placental lactogen, tumor necrosis factor, tumor necrosis factor-α, tumor necrosis factor-β, mullerian inhibiting substance, mouse gonadotropin-associated peptide, inhibin, activin, vascular endothelial growth factor, thrombopoietin, erythropoietin, osteoinductive factor, interferon, interferon-α, interferon-β, interferon-γ, colony stimulating factor (CSF), macrophage-CSF, granulocyte-macrophage-CSF, granulocyte-CSF, interleukin (IL), IL-1, IL-1α, IL-2, IL-3, IL-4, IL-5, IL-6, IL-7, IL-8, IL-9, IL-10, IL-11, IL-12, tumor necrosis factor, polypeptide factor, LIF, kit ligand, or a combination thereof;
(c) diphtheria toxin, botulinum toxin, tetanus toxin, decentretoxin, cholera toxin, amanitin, α-amanitin, pyrrolobenzodiazepines, pyrrolobenzodiazepine derivatives, indolinobenzodiazepines, pyridinobenzodiazepines, tetrodotoxin, brevetoxin, ciguatoxin, ricin, AM toxin, auristatin, tubulysin, geldanamycin, maytansinoid, calicheamycin, daunomycin, doxorubicin, methotrexate, vindesine, SG2285, dolastatin, dolastatin analog, auristatin, cryptophycin, camptothecin, rhizoxin, rhizoxin derivatives, CC-1065, CC-1065, analogs or derivatives, duocarmycin, enediyne antibiotics, esperamicin, epothilone, toxoid, or a combination thereof;
(d) an affinity ligand, wherein the affinity ligand is a substrate, an inhibitor, an active agent, a neurotransmitter, a radioisotope, or a combination thereof;
(e) a radioactive label, 32P, 35S, a fluorescent dye, an electron dense reagent, an enzyme, biotin, streptavidin, dioxigenin, hapten, an immunogenic protein, a nucleic acid molecule with a sequence complementary to a target, or a combination thereof;
(f) an immunomodulatory compound, an anticancer agent, an anti-viral agent, an anti-bacterial agent, an anti-fungal agent, an anti-parasitic agent, or a combination thereof;
(g) tamoxifen, raloxifene, droloxifene, 4-hydroxytamoxifen, trioxifene, keoxifene, LY117018, onapristone, or toremifene;
(h) 4 (5)-imidazole, aminoglutethimide, megestrol acetate, exemestane, letrozole, or anastrozole;
(i) flutamide, nilutamide, bicalutamide, leuprolide, goserelin, or troxacitabine;
(j) aromatase inhibitors;
(k) protein kinase inhibitors;
(l) lipid kinase inhibitors;
(m) antisense oligonucleotides;
(n) ribozymes;
(o) vaccines; and
(p) anti-angiogenic agents.

78. The conjugate of any one of claims 1-77, wherein:
the active agent is a pyrrolobenzodiazepine dimer;
position N10 of the pyrrolobenzodiazepine dimer is substituted with X or position N′10 is substituted with X′, wherein X or X′ links the pyrrolobenzodiazepine dimer to the linker;
X and X′ are each independently —C(O)O—* or —C(O)—*; and
* refers to a binding site between the pyrrolobenzodiazepine dimer and the linker.

79. The conjugate of claim 78, wherein the pyrrolobenzodiazepine dimer is represented by Formula III: 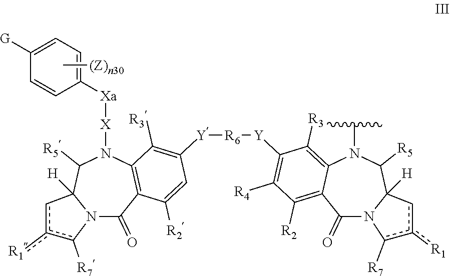
wherein
the wavy line indicates a connection point to the conjugate (e.g., the carbonyl of formula IIa connected to B′);
a dotted line represents an optional double bond;
R₁ and R₁′ are each independently selected from H, OH, ═O, ═CH₂, CN, R^{m}, OR^{m}, ═CH—R^{m′} ═C(R^{m′})₂, O—SO₂—R^{m}, CO₂R^{m}, COR^{m}, halo, and dihalo;
R^{m′} is selected from R^{m}, CO₂R^{m}, COR^{m}, CHO, CO₂H, and halo;
R^{m} is selected from substituted or unsubstituted C₁₋₁₂ alkyl, substituted or unsubstituted C₂₋₁₂ alkenyl, substituted or unsubstituted C₂₋₁₂ alkynyl, substituted or unsubstituted C₅₋₂₀ aryl, substituted or unsubstituted C₃₋₆ heteroaryl, substituted or unsubstituted C₃₋₆ cycloalkyl, substituted or unsubstituted 3- to 7-membered heterocyclyl, substituted or unsubstituted 3- to 7-membered heterocycloalkyl, and substituted or unsubstituted 5- to 7-membered heteroaryl, wherein when the C₁₋₁₂ alkyl, C₂₋₁₂ alkenyl, C₂₋₁₂ alkynyl, C₅₋₂₀ aryl, C₅₋₂₀ heteroaryl, C₃₋₆ cycloalkyl, 3- to 7-membered heterocyclyl, 3- to 7-membered heterocycloalkyl, or 5- to 7-membered heteroaryl is substituted, the respective hydrogen atoms in the C₁₋₁₂ alkyl, C₂₋₁₂ alkenyl, C₂₋₁₂ alkynyl, C₅₋₂₀ aryl, C₅₋₂₀ heteroaryl, C₃₋₆ cycloalkyl, 3- to 7-membered heterocyclyl, 3- to 7-membered heterocycloalkyl, or 5- to 7-membered heteroaryl may each be independently replaced with methoxy, C₁₋₁₂ alkyl, C₂₋₁₂ alkenyl, C₂₋₁₂ alkynyl, C₅₋₂₀ aryl, C₅₋₂₀ heteroaryl, C₃₋₆ cycloalkyl, 3- to 7-membered heterocyclyl, 3- to 7-membered heterocycloalkyl, and 5- to 7-membered heteroaryl;
R₂, R₃, R₅, R₂′, R₃′, and R₅′ are each independently selected from H, R^{m}, OH, OR^{m}, SH, SR^{m}, NH₂, NHR^{m}, NR^{m}R^{m′}, NO₂, Me₃Sn, and halo;
R₄ and R₄′ are each independently selected from H, R^{m}, OH, OR^{m}, SH, SR^{m}, NH₂, NHR^{m}, NR^{m}R^{m′}, NO₂, Me₃Sn, halo, substituted or unsubstituted C₁₋₆ alkyl, substituted or unsubstituted C₁₋₆ alkoxy, substituted or unsubstituted C₂₋₆ alkenyl, substituted or unsubstituted C₂₋₆ alkynyl, substituted or unsubstituted C₃₋₆ cycloalkyl, substituted or unsubstituted 3- to 7-membered heterocycloalkyl, substituted or unsubstituted C₅₋₁₂ aryl, substituted or unsubstituted 5- to 7-membered heteroaryl, —CN, —NCO, —ORⁿ, —OC(O)Rⁿ, —OC(O)NRⁿR^{n′}, —OS(O)Rⁿ, —OS(O)₂Rⁿ, —SRⁿ, —S(O)Rⁿ, —S(O)₂Rⁿ, —S(O)NRⁿR^{n′}, —S(O)₂NRⁿR^{n′}, —OS(O)NRⁿR^{n′}, —OS(O)₂NRⁿR^{n′}, —NRⁿR^{n′}, —NRⁿC(O)R^{o}, —NRⁿC(O)OR^{o}, —NRⁿC(O)NR^{o}R^{o′}, —NRⁿS(O)R^{o}, —NRⁿS(O)₂R^{o}, —NRⁿS(O)NR^{o}R^{o′}, —NRⁿS(O)₂NR^{o}R^{o′}, —C(O)Rⁿ, —C(O)ORⁿ, and —C(O)NRⁿR^{n′}, wherein the hydrogen atoms in the C₁₋₆ alkyl, C₁₋₆ alkoxy, C₂₋₆ alkenyl, C₂₋₆ alkynyl, C₃₋₆ cycloalkyl, 3- to 7-membered heterocycloalkyl, C₅₋₁₂ aryl, and 5- to 7-membered heteroaryl may each be independently replaced with C₁₋₆ alkyl, C₁₋₆ alkoxy, C₂₆ alkenyl, C₂₋₆ alkynyl, C₃₋₆ cycloalkyl, 3- to 7-membered heterocycloalkyl, C₅₋₁₂ aryl, 5- to 7-membered heteroaryl, —OR^{p}, —OC(O)R^{p}, —OC(O)NR^{p}R^{p′}, —OS(O)R^{p}, —OS(O)₂R^{p}, —SR^{p}, —S(O)R^{p}, —S(O)₂R^{p}, —S(O)NR^{p}R^{p′}, —S(O)₂NR^{p}R^{p′}, —OS(O)NR^{p}R^{p′}, —OS(O)₂NR^{p}R^{p′}, —NR^{p}R^{p′}, —NR^{p}C(O)R^{q}, —NR^{p}C(O)OR^{q}, —NR^{p}C(O)NR^{q}H, —NR^{p}S(O)R^{q}, —NR^{p}S(O)₂R^{q}, —NR^{p}S(O)NR^{q}H, —NR^{p}S(O)₂NR^{q}H, —C(O)R^{p}, —C(O)OR^{p}, or —C(O)NR^{p}R^{p} when the C₁₋₆ alkyl, C₁₋₆ alkoxy, C₂₋₆ alkenyl, C₂₋₆ alkynyl, C₃₋₆ cycloalkyl, 3- to 7-membered heterocycloalkyl, C₅₋₁₂ aryl, and 5- to 7-membered heteroaryl;
Rⁿ, R^{n′}, R^{o}, R^{o′} R^{p}, R^{p′}, and R^{q} are each independently selected from H, C₁₋₇ alkyl, C₂₋₇ alkenyl, C₂₋₇ alkynyl, C₃₋₁₃ cycloalkyl, 3- to 7-membered heterocycloalkyl, C₆₋₁₀ aryl, and 5- to 7-membered heteroaryl;
X is selected from —C(O)O—, —S(O)O—, —C(O)—, —C(O)NR—, —S(O)₂NR—, —P(O)R′NR—, —S(O)NR—, and —PO₂NR—;
Xa is a bond or substituted or unsubstituted C₁₋₆ alkylene, wherein C₁₋₆ alkylene is substituted with C₁₋₈ alkyl, or C₃₋₈ cycloalkyl when substituted;
R and R′ each independently denote H, OH, NH₂, ONH₂, NHNH₂, substituted or unsubstituted C₁₋₈ alkyl, substituted or unsubstituted C₃₋₈ cycloalkyl, substituted or unsubstituted C₁₋₈ alkoxy, substituted or unsubstituted C₁₋₈ alkylthio, substituted or unsubstituted C₃₋₂₀ heteroaryl, substituted or unsubstituted C₅₋₂₀ aryl, or mono- or di-C₁₋₈ alkylamino, wherein the C₁₋₈ alkyl, C₃₋₈ cycloalkyl, C₁₋₈ alkoxy, C₁₋₈ alkylthio, C₃₋₂₀ heteroaryl, and C₅₋₂₀ aryl are substituted with a substituent selected from OH, N3, CN, NO₂, SH, NH₂, ONH₂, NHNH₂, halo, C₁₋₆ alkyl, C₁₋₆ alkoxy, and C₆₋₁₂ aryl when substituted;
Y and Y′ are each independently selected from O, S, and N(H);
R₆ is a substituted or unsubstituted saturated or unsaturated C₃₋₁₂ hydrocarbon chain, wherein the chain may be interrupted by one or more heteroatoms, NMe, or a substituted or unsubstituted aromatic ring, the chain or aromatic ring may be substituted with —NH, —NR^{m}, —NHC(O)R^{m}, —NHC(O)CH₂—[OCH₂CH₂]ₙ—R, or —[CH₂CH₂O]ₙ—R at any one or more positions of hydrogen atoms on the chain or aromatic ring or unsubstituted, wherein R^{m} and R are each as defined for R^{m} and R above, and n is 1 to 12; and
R₇ and R₇ are each independently H, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ cycloalkyl, substituted or unsubstituted 3- to 7-membered heterocycloalkyl, substituted or unsubstituted C₆₋₁₀ aryl, substituted or unsubstituted 5- to 7-membered heteroaryl, —OR^{r}, —OC(O)R^{r}, —OC(O)NR^{r}R^{r′}, —OS(O)R^{r}, —OS(O)₂R^{r}, —SR^{r}, —S(O)R^{r}, —S(O)₂R^{r}, —S(O)NR^{r}R^{r′}, —S(O)₂NR^{r}R^{r′}, —OS(O)NR^{r}R^{r′}, —OS(O)₂NR^{r}R^{r′}, —NR^{r}R^{r′}, —NR^{r}C(O)R^{s}, —NR^{r}C(O)OR^{s}, —NR^{r}C(O)NR^{s}R^{s′}, —NR^{r}S(O)R^{s}, —NR^{r}S(O)₂R^{s}, —NR^{r}S(O)NR^{s}R^{s′}, —NR^{r}S(O)₂NR^{s}R^{s}, —C(O)R^{r}, —C(O)OR^{s}, or —C(O)NR^{r}R^{r′}, wherein the hydrogen atoms in the C₁₋₆ alkyl, C₂₋₆ alkenyl, C₂₋₆ alkynyl, C₃₋₆ cycloalkyl, 3- to 7-membered heterocycloalkyl, C₆₋₁₀ aryl, and 5- to 7-membered heteroaryl may each be independently replaced with C₁₋₆ alkyl, C₂₋₆ alkenyl, C₂₋₆ alkynyl, C₃₋₆ cycloalkyl, 3- to 7-membered heterocycloalkyl, C₆₋₁₀ aryl, 5- to 7-membered heteroaryl, —OR′, —OC(O)R^{t}, —OC(O)NR^{t}R^{t′}, —OS(O)R^{t}, —OS(O)₂R^{t}, —SR^{t}, —S(O)R^{t}, —S(O)₂R^{t}, —S(O)NR^{t}R^{t′}, —S(O)₂NR^{t}R^{t′}, —OS(O)NR^{t}R^{t′}, —OS(O)₂NR^{t}R^{t′}, —NR^{t}R^{t′}, —NR^{t}C(O)R^{u}, —NR^{t}C(O)OR^{u}, —NR^{t}C(O)NR^{u}R^{u′}, —NR^{t}S(O)R^{u}, —NR^{t}S(O)₂R^{u}, —NR^{t}S(O)NR^{u}R^{u′}, —NR^{t}S(O)₂NR^{u}R^{u′}, —C(O)R^{t}, —C(O)OR^{t}, or —C(O)NR^{t}R^{t′} when the C₁₋₆ alkyl, C₂₋₆ alkenyl, C₂₋₆ alkynyl, C₃₋₆ cycloalkyl, 3- to 7-membered heterocycloalkyl, C₆₋₁₀ aryl, and 5- to 7-membered heteroaryl;
R^{r}, R^{r′}, R^{s}, R^{s′}, R^{t}, R^{t′}, R^{u}, and R^{u′} are each independently selected from H, C₁₋₇ alkyl, C₂₋₇ alkenyl, C₂₋₇ alkynyl, C₃₋₁₃ cycloalkyl, 3- to 7-membered heterocycloalkyl, C₅₋₁₀ aryl, and 5- to 7-membered heteroaryl;
G is a glucuronide group or a galactoside group;
each Z is selected from H, C₁₋₈ alkyl, halo, NO₂, CN, 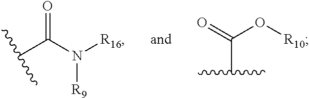
R₉, R₁₀, and R₁₆ are each independently selected from H, C₁₋₈ alkyl, C₂₋₆ alkenyl, C₁₋₆ alkoxy, alkyloxyalkyl, and methyloxyethyl; and
n30 is 1 to 3.

80. The conjugate claim 79, wherein Y is O.

81. The conjugate claim 79 or 80, wherein is Y′ is O.

82. The conjugate of any one of claims 79-81, wherein a dotted line represents presence of a double bond between the carbons bearing R₁ and R₇ or R_{1′} and R_{7′}.

83. The conjugate of any one of claims 79-82, wherein R₁ is R^{m} and R^{m} is selected from substituted or unsubstituted C₁₋₆ alkyl, substituted or unsubstituted C₂₋₆ alkenyl, substituted or unsubstituted C₅₋₇ aryl, and substituted or unsubstituted C₃₋₆ heteroaryl.

84. The conjugate of any one of claims 79-83, wherein R₂, R₃, and R₅ are each independently H or OH.

85. The conjugate of any one of claims 79-84, wherein R₄ is C₁₋₆ alkoxy.

86. The conjugate of any one of claims 79-84, wherein R₄ is methoxy, ethoxy, or butoxy.

87. The conjugate of any one of claims 79-86, wherein
X is selected from —C(O)O—, —C(O)—, and —C(O)NR—; and
Rs each independently denote H, OH, N₃, CN, NO₂, SH, NH₂, ONH₂, NHNH₂, halo, substituted or unsubstituted C₁₋₈ alkyl, or substituted or unsubstituted C₁₋₈ alkoxy, wherein C₁₋₈ alkyl or C₁₋₈ alkoxy is substituted with OH, N₃, CN, NO₂, SH, NH₂, ONH₂, NNH₂, or halo when substituted.

88. The conjugate of any one of claims 79-87, wherein X is —C(O)NR—.

89. The conjugate of any one of claims 79-88, wherein R₆ is a substituted or unsubstituted saturated or unsaturated C₃₋₈ hydrocarbon chain, wherein
one or more of the carbon atoms of the hydrocarbon chain is replaced by a heteroatom or a substituted or unsubstituted aromatic ring, wherein the heteroatom is O, S, or N(H) and the aromatic ring is benzene, pyridine, imidazole, or pyrazole, and the chain or aromatic ring may be substituted with —NHC(O)CH₂—[OCH₂CH₂]ₙ—R or —[CH₂CH₂O]ₙ—R at any one or more positions of hydrogen atoms on the chain or aromatic ring; and
n is 1 to 6.

90. The conjugate of any one of claims 79-89, wherein n is 1 to 10.

91. The conjugate of any one of claims 79-90, wherein Xa is a bond or C₁₋₃ alkylene.

92. The conjugate of any one of claims 99-110, wherein Z is H, 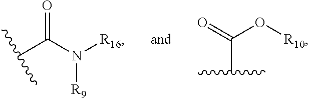
wherein R₉, R₁₀, and R₁₆ are each independently selected from H, C₁₋₃ alkyl, C₁₋₃ alkoxy, and allyloxymethyl.

93. The conjugate of claim 92, wherein R₉ is methyloxyalkyl.

94. The conjugate of claim 92 or 93, wherein R₁₀ is methyloxyalkyl.

95. The conjugate of any one of claims 92-94, wherein R₁₆ is methyloxyalkyl.

96. The conjugate of any one of claims 92-95, wherein R₉, R₁₀, or R₁₆ is —(CH₂CH₂O)ₘ—(CH₂)ₘ₂CH₃, further wherein m is 1-6 and m2 is 0-2.

97. The conjugate of any one of claims 92-95, wherein m is 1.

98. The conjugate of any one of claims 92-95, wherein m2 is 1.

99. The conjugate of claim 79, wherein R₂ is H.

100. The conjugate of claim 99, wherein R₃ is H.

101. The conjugate of claim 99 or 100, wherein R₇ is H.

102. The conjugate of any one of claims 99-101, wherein R₄ is alkoxy (e.g., methoxy).

103. The conjugate of any one of claims 99-102, wherein R₅ is OH.

104. The conjugate of any one of claims 99-103, wherein R₁ is ═CH₂, CH₃, or phenyl, optionally substituted with methoxy.

105. The conjugate of any one of claims 99-104, wherein Y is O.

106. The conjugate of any one of claims 99-105, wherein R₂′ is H.

107. The conjugate of any one of claims 99-106, wherein R₃′ is H.

108. The conjugate of any one of claims 99-107, wherein R₇′ is H.

109. The conjugate of any one of claims 99-108, wherein R₄′ is alkoxy (e.g., methoxy).

110. The conjugate of any one of claims 99-109, wherein R₅′ is OH.

111. The conjugate of any one of claims 99-110, wherein R₁′ is ═CH₂, CH₃, or phenyl, optionally substituted with methoxy.

112. The conjugate of any one of claims 99-111, wherein Y′ is O.

113. The conjugate of any one of claims 99-112, wherein X is —C(O)O—

114. The conjugate of any one of claims 99-113, wherein Xa is CH₂.

115. The conjugate of any one of claims 99-114, wherein G is a glucuronide group.

116. The conjugate of claim 115, wherein G is

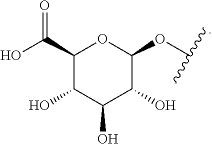 [SMILES: *O[C@@H]1O[C@H](C(=O)O)[C@@H](O)[C@H](O)[C@H]1O]

117. The conjugate of any one of claims 99-116, wherein n30 is 1.

118. The conjugate of any one of claims 99-117, wherein Z is

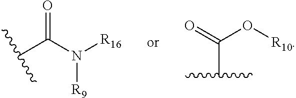

119. The conjugate of claim 118, wherein R₉ is H.

120. The conjugate of claim 118 or 119, wherein R₁₆ is alkyloxyalkyl (e.g., methoxyethyl).

121. The conjugate of any one of claims 99-117, wherein Z is

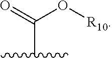

122. The conjugate of claim 121, wherein R₁₀ is alkyl (e.g., methyl).

123. The conjugate of claim 121, wherein R₆ is alkyl (e.g., pentyl).

124. The conjugate of any one of claims 1-123, wherein the conjugate comprises a structure selected from: 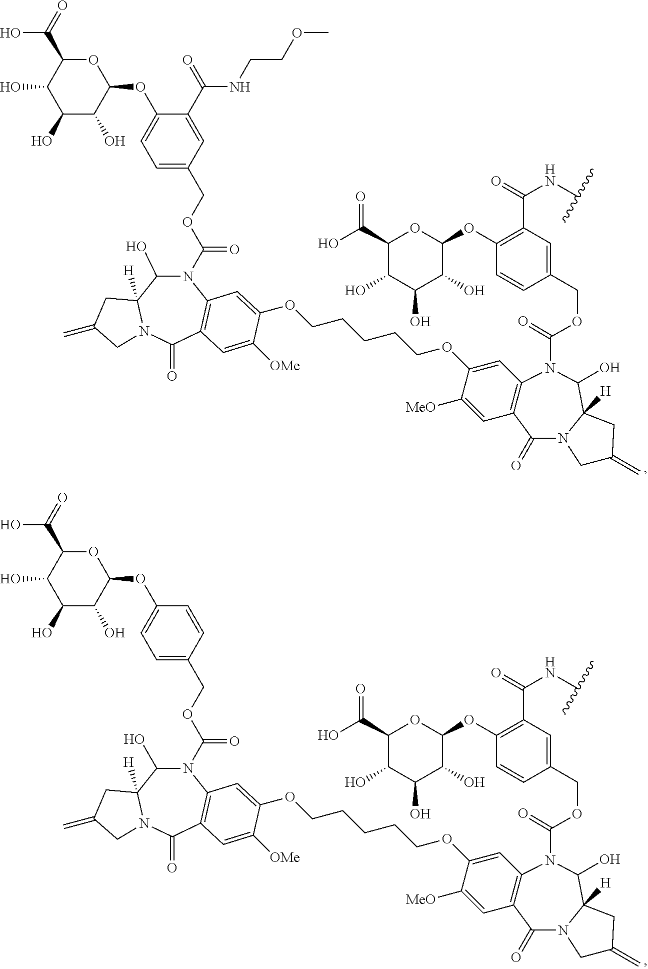 [SMILES: *NC(=O)c1cc(COC(=O)N2c3cc(OCCCCCOc4cc5c(cc4OC)C(=O)N4CC(=C)C[C@H]4C(O)N5C(=O)OCc4ccc(O[C@@H]5O[C@H](C(=O)O)[C@@H](O)[C@H](O)[C@H]5O)c(C(=O)NCCOC)c4)c(OC)cc3C(=O)N3CC(=C)C[C@H]3C2O)ccc1O[C@@H]1O[C@H](C(=O)O)[C@@H](O)[C@H](O)[C@H]1O ; *NC(=O)c1cc(COC(=O)N2c3cc(OCCCCCOc4cc5c(cc4OC)C(=O)N4CC(=C)C[C@H]4C(O)N5C(=O)OCc4ccc(O[C@@H]5O[C@H](C(=O)O)[C@@H](O)[C@H](O)[C@H]5O)cc4)c(OC)cc3C(=O)N3CC(=C)C[C@H]3C2O)ccc1O[C@@H]1O[C@H](C(=O)O)[C@@H](O)[C@H](O)[C@H]1O] 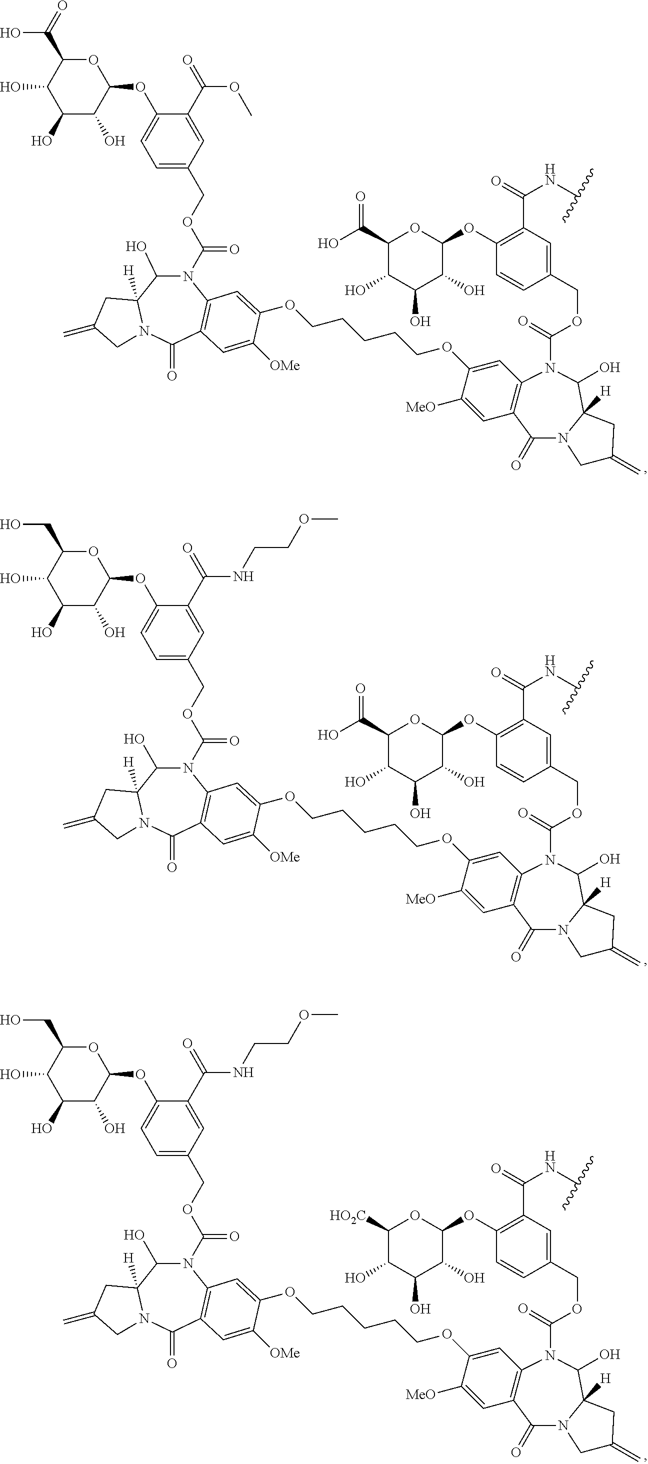 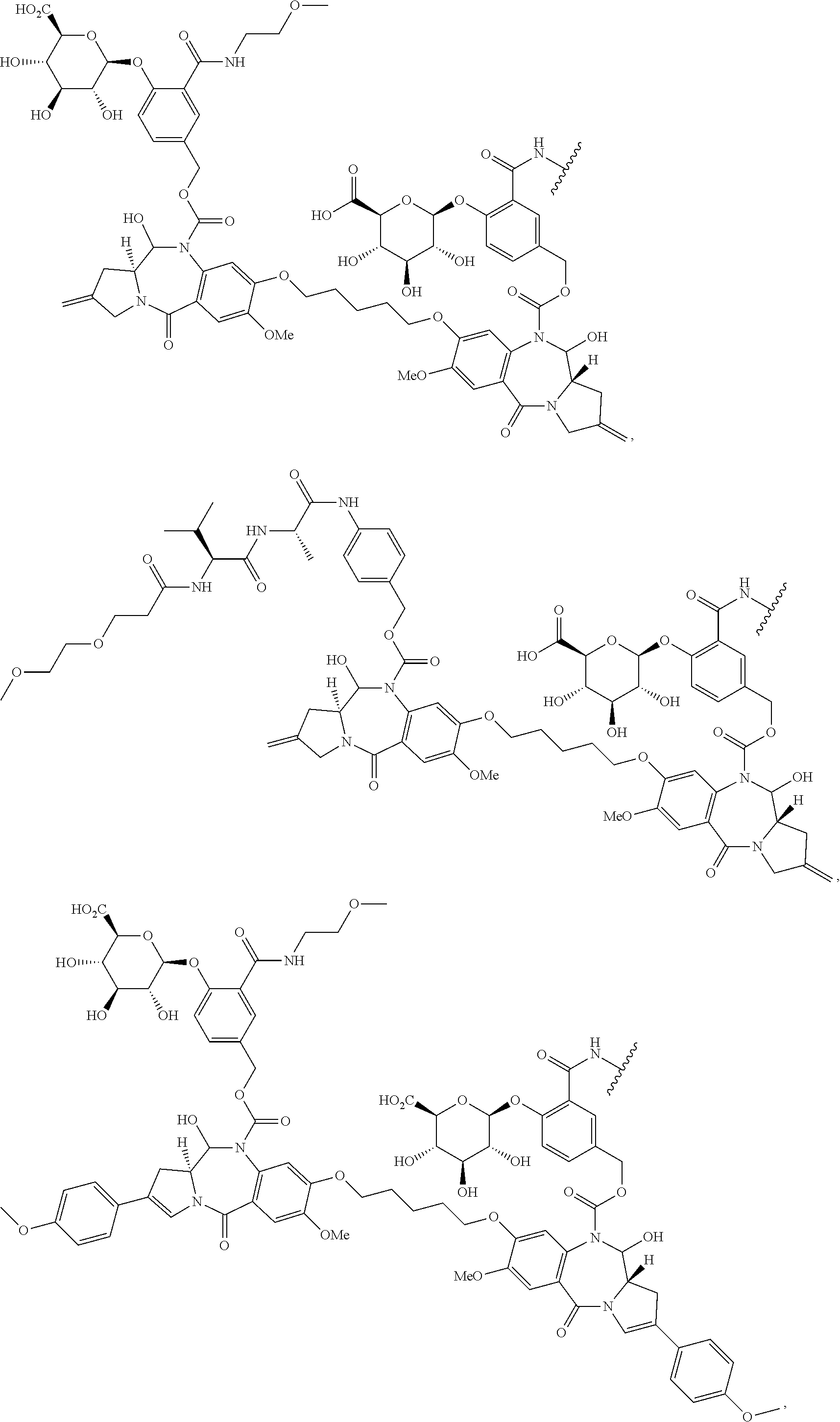 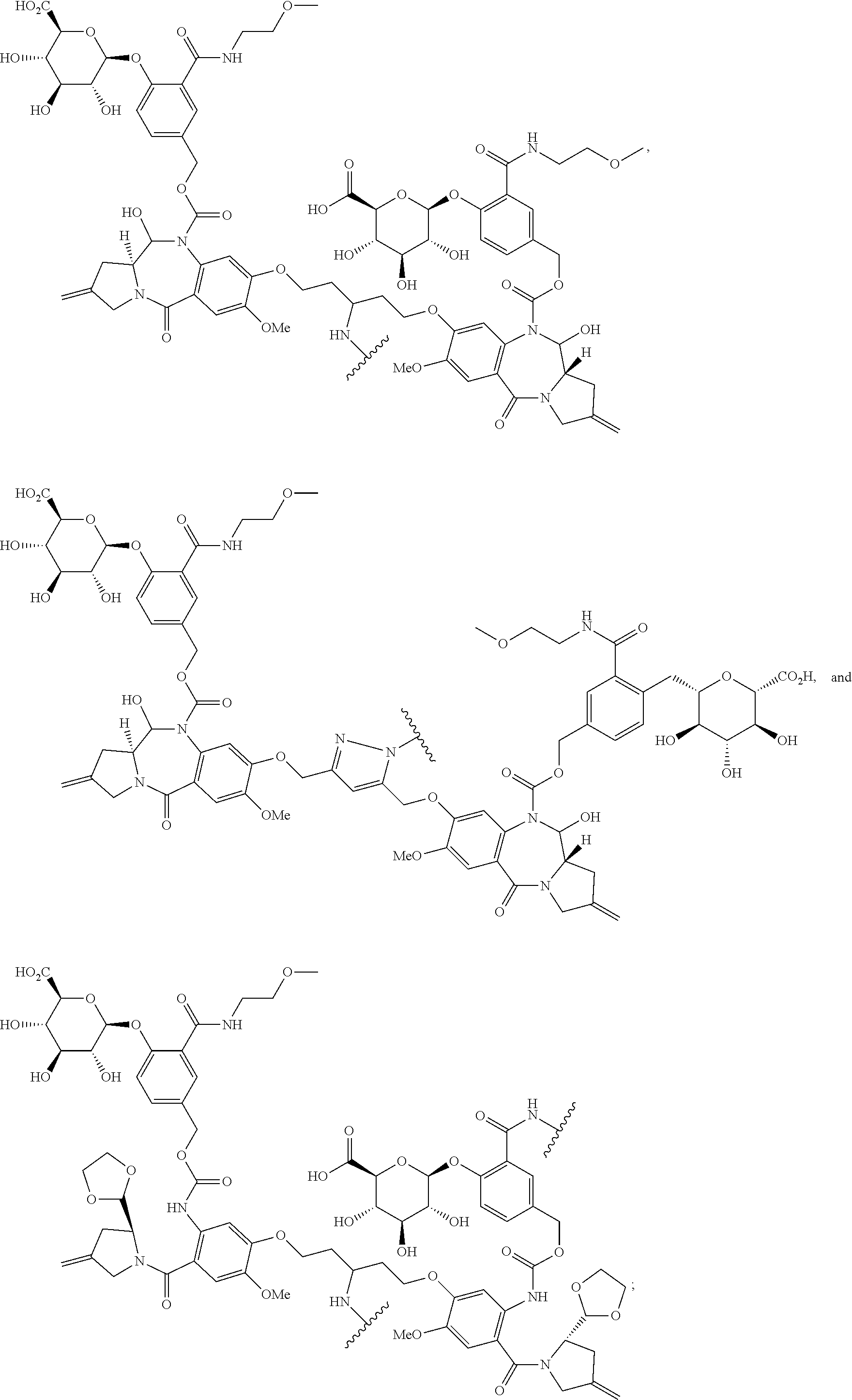
and the bond overlaid with a wavy line represents a connection point to L.

125. The conjugate any one of claims 1-124, wherein the conjugate comprises a structure selected from: 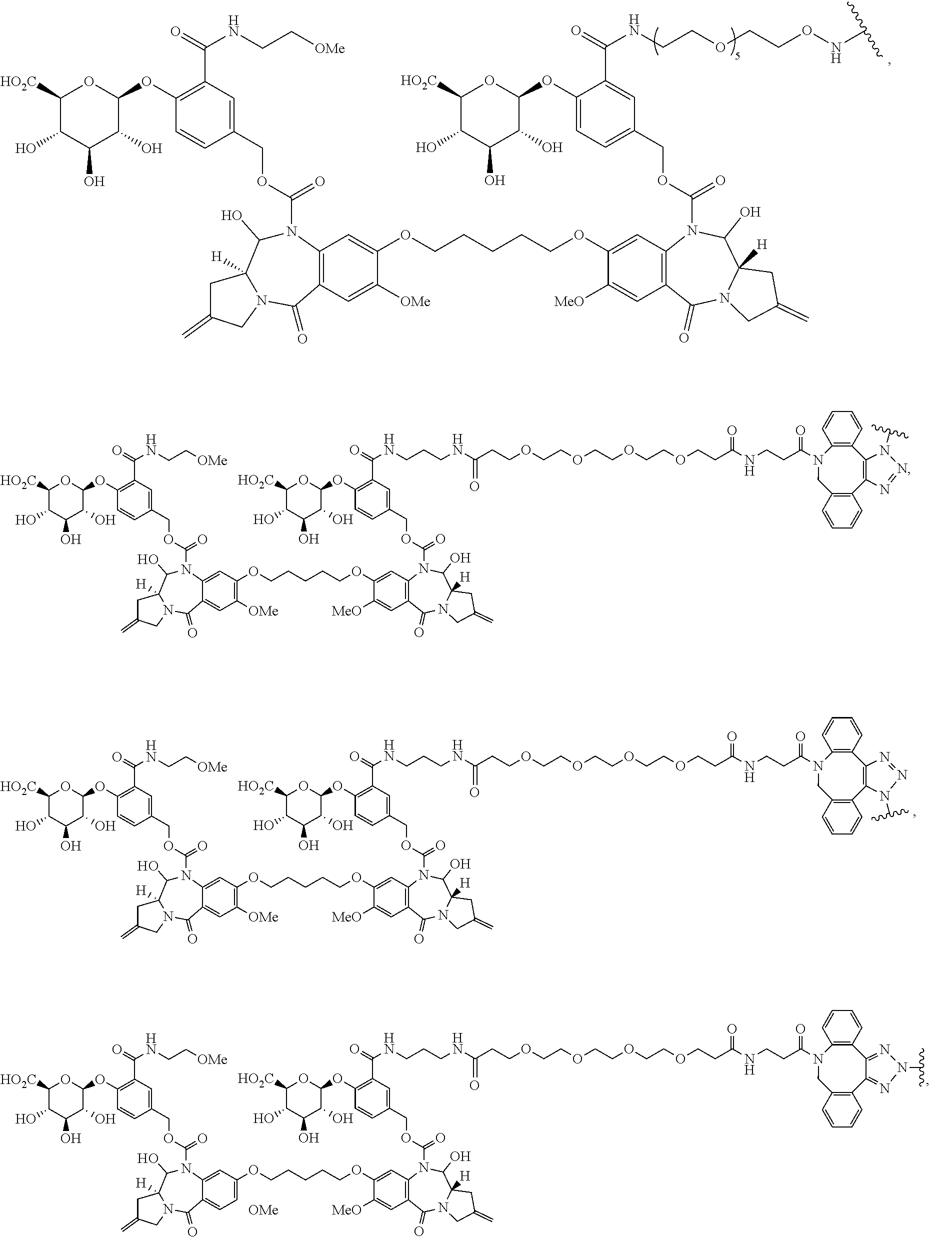 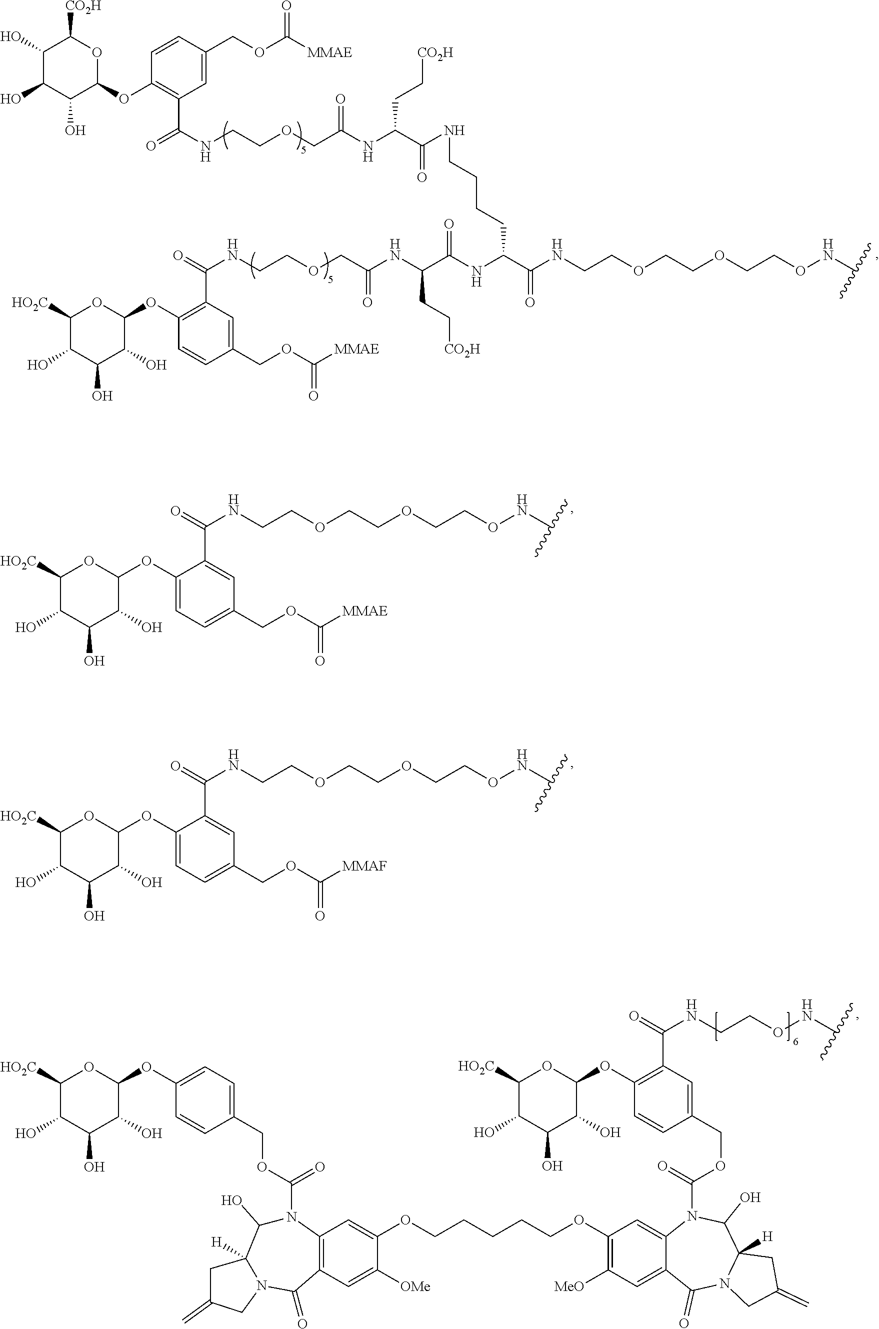 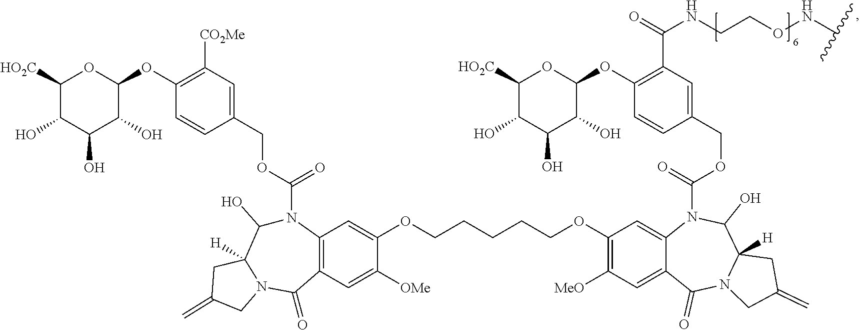 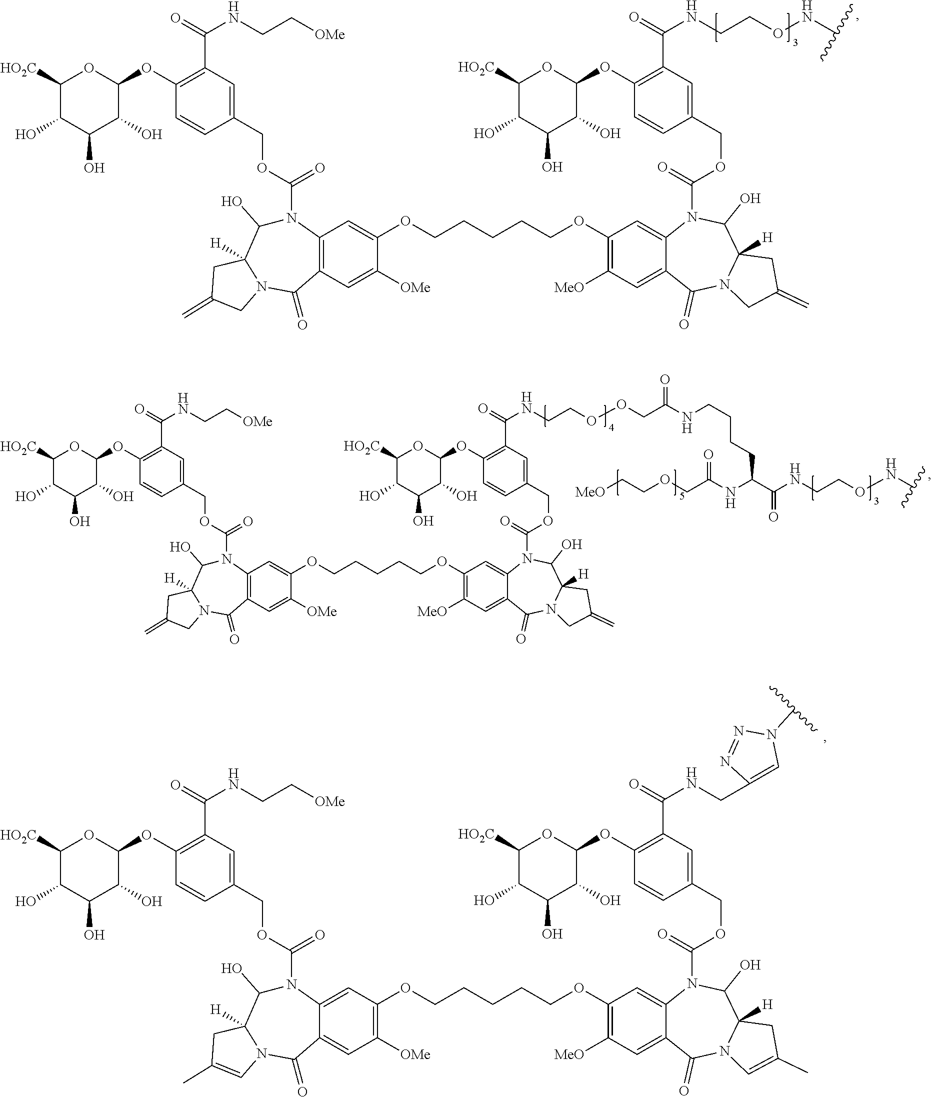 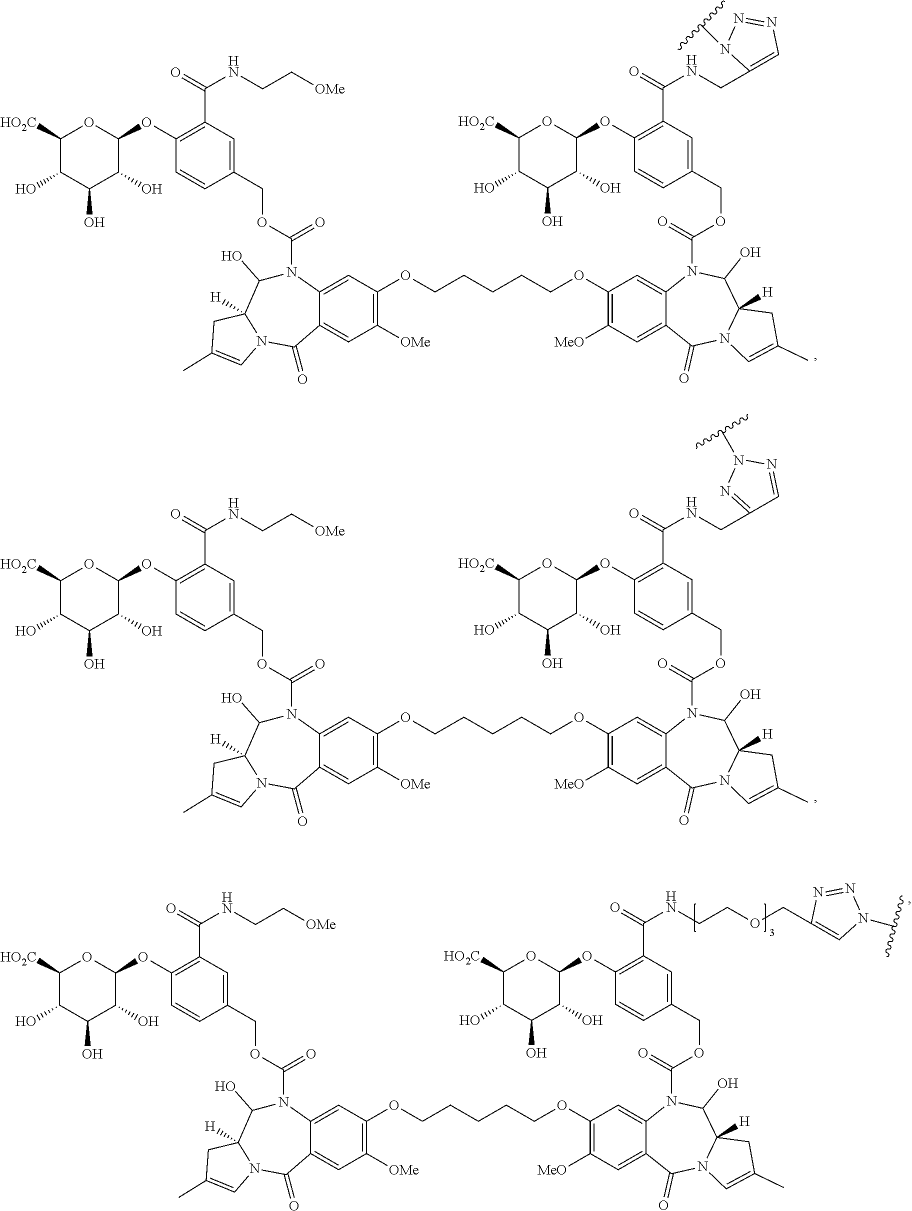 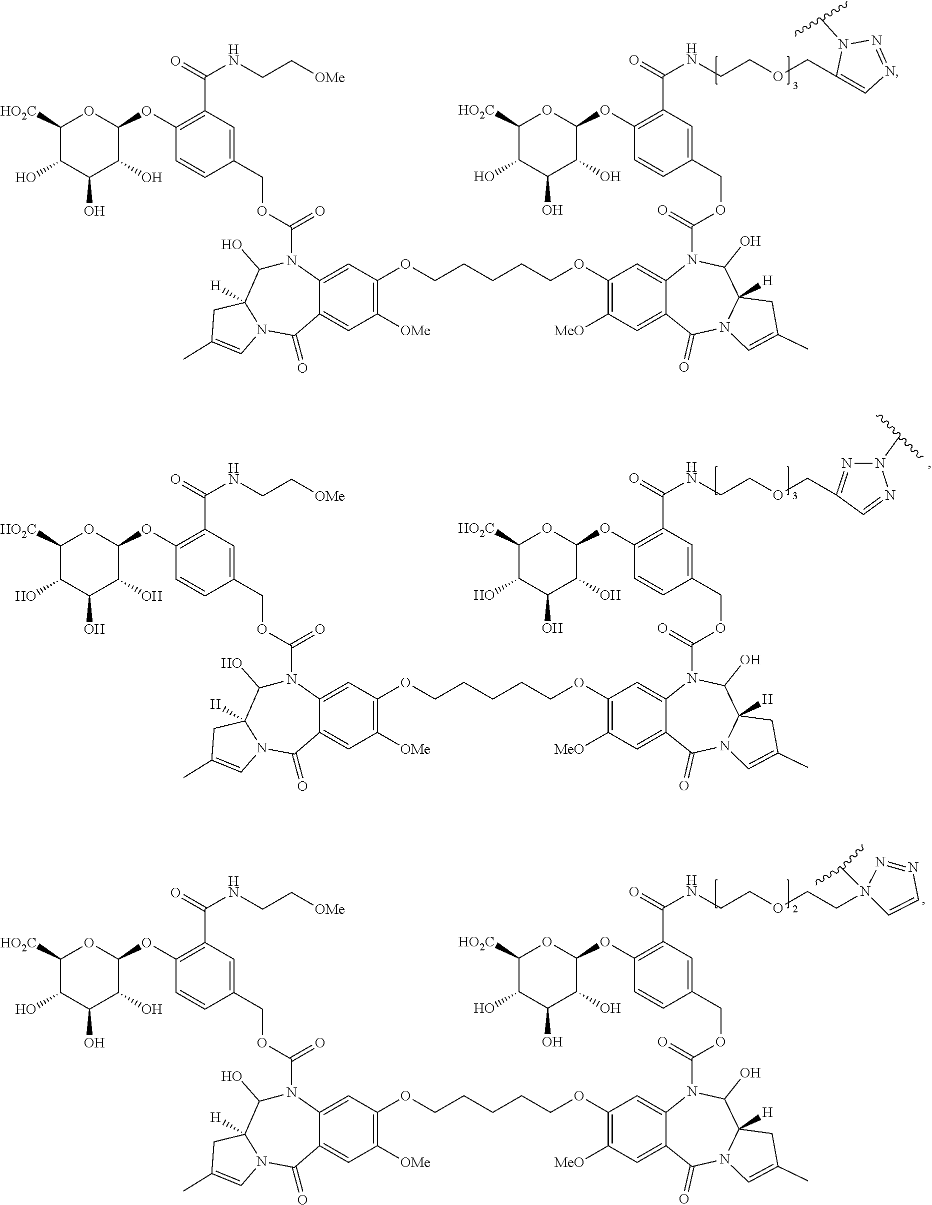 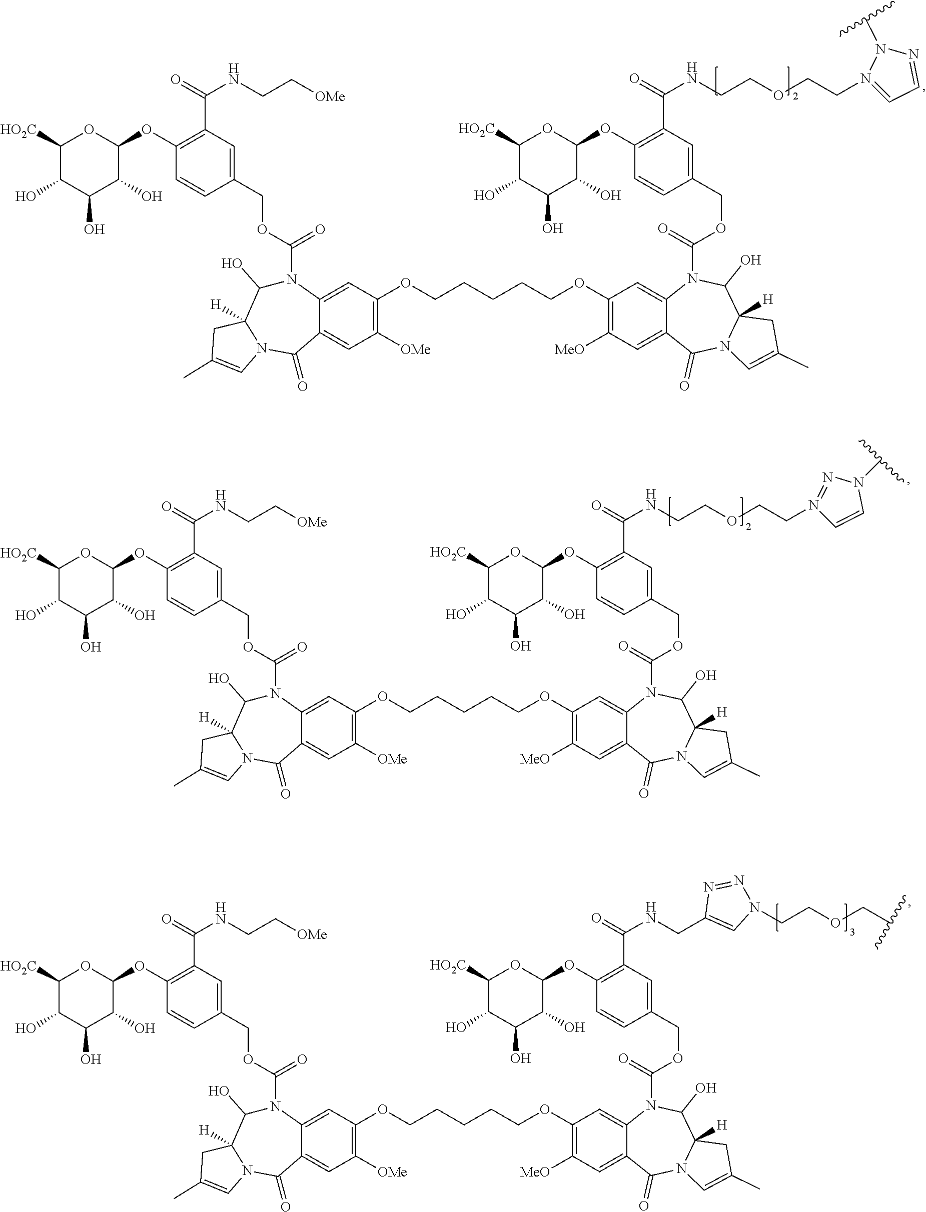 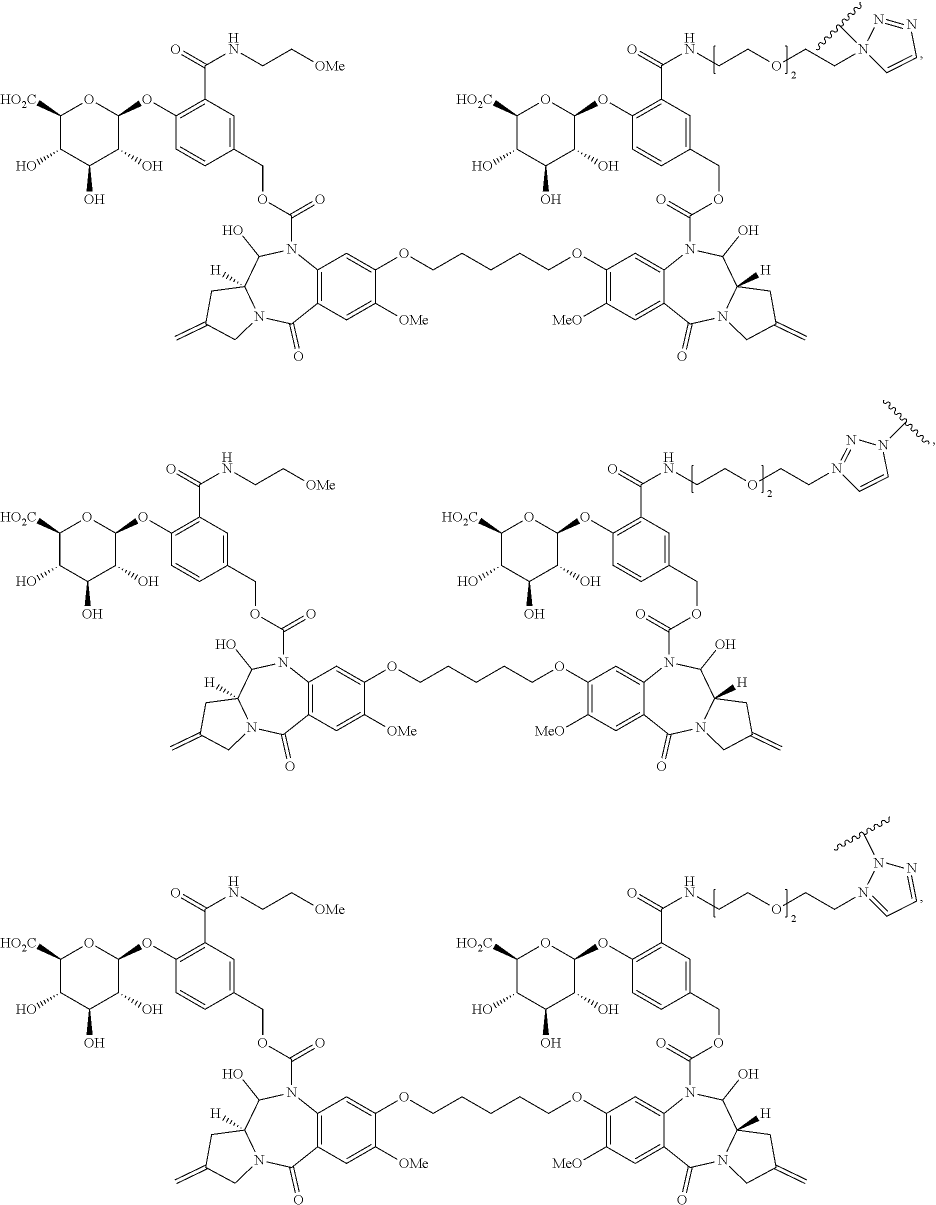 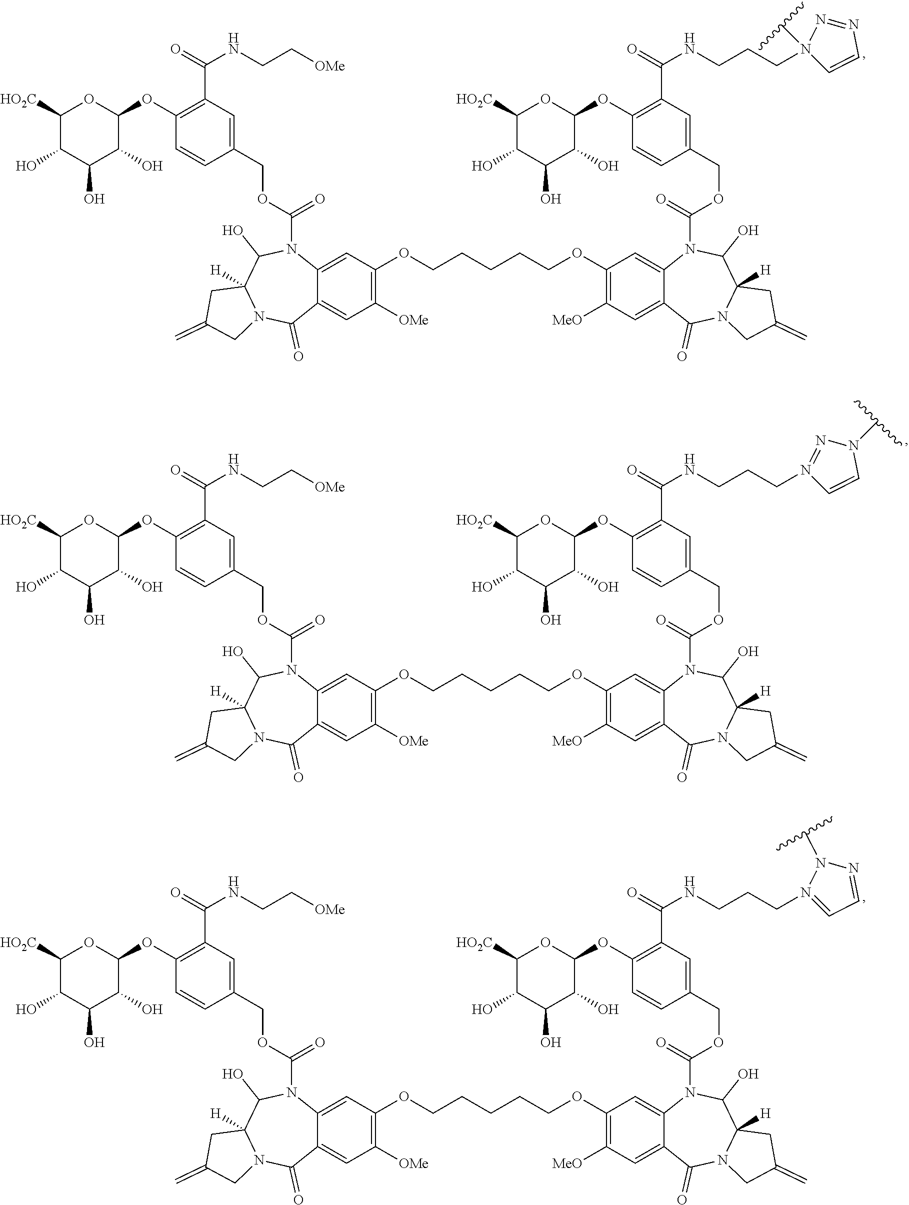 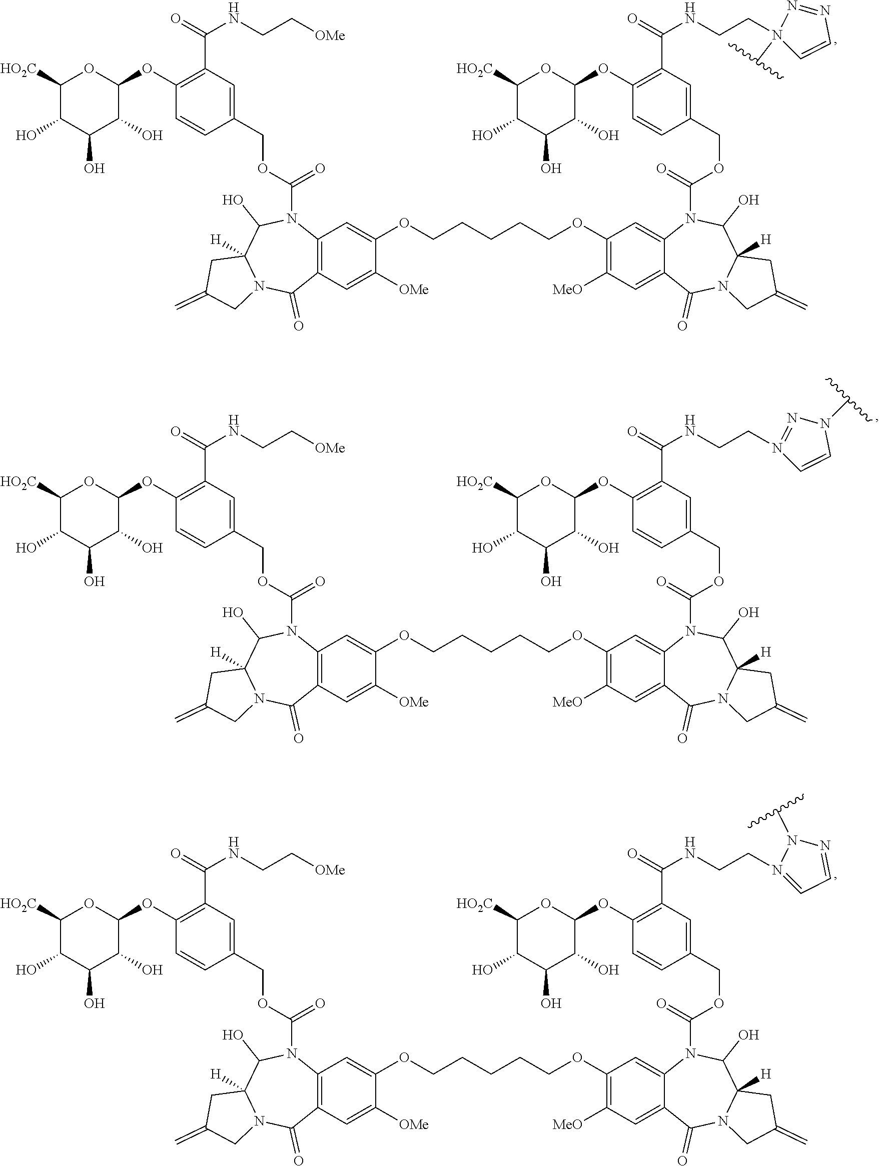 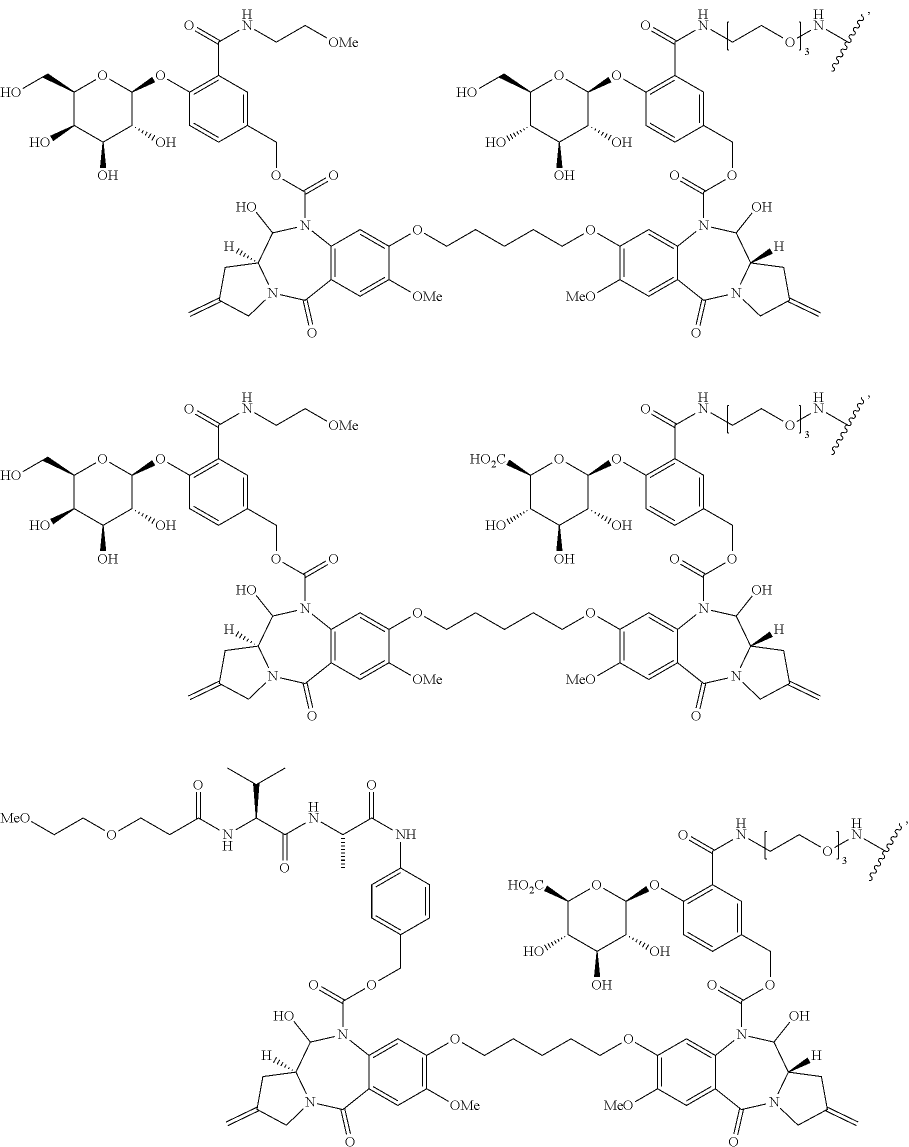 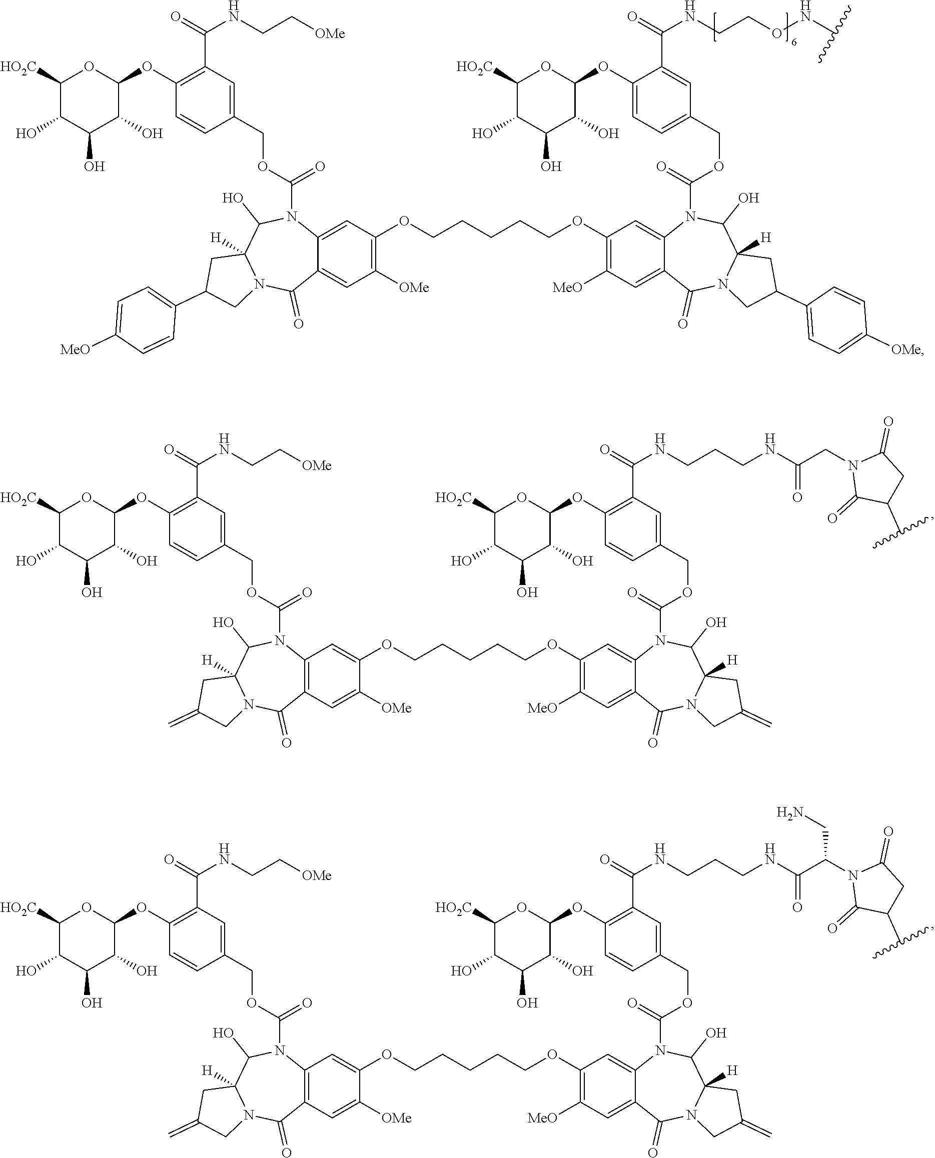 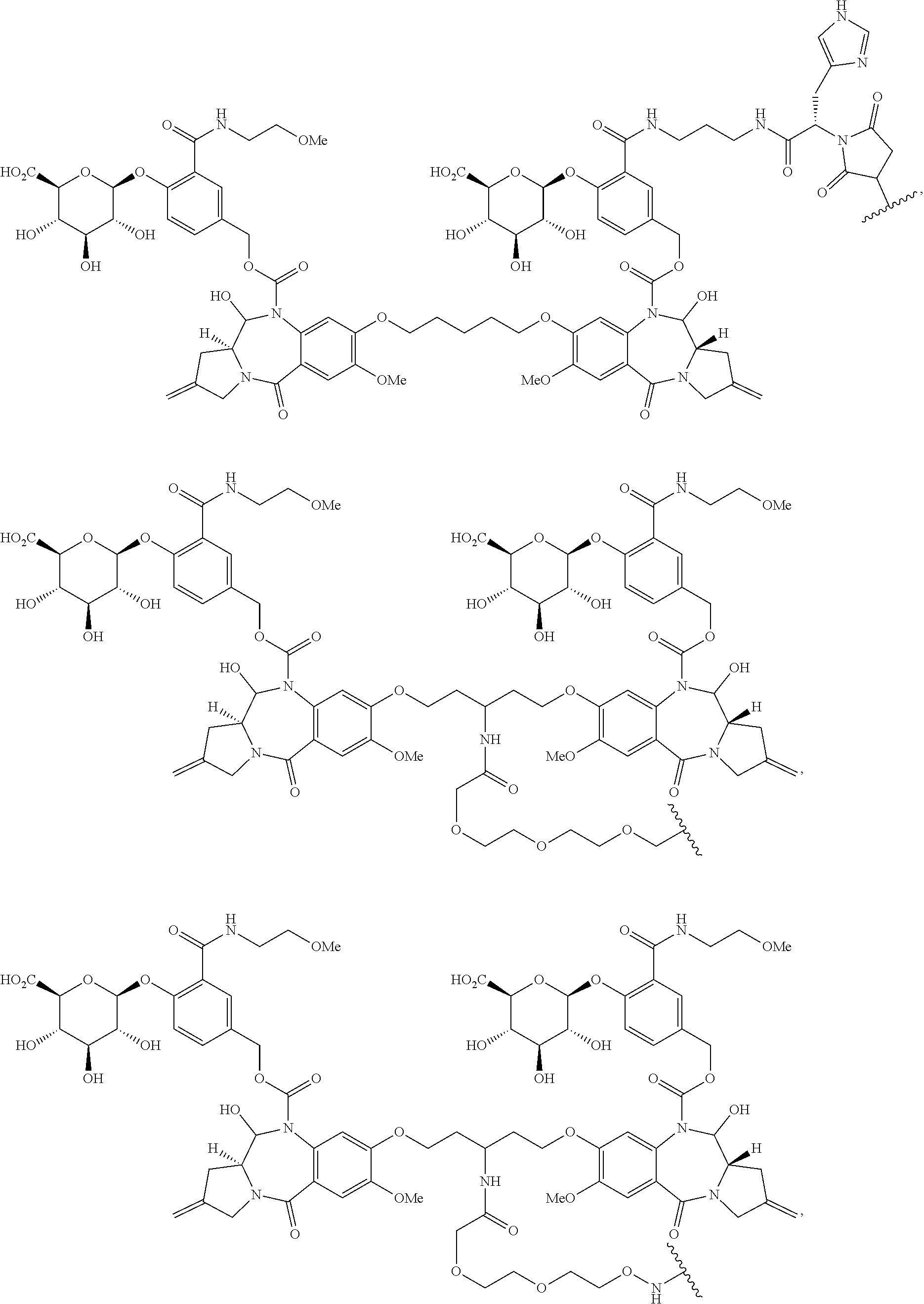 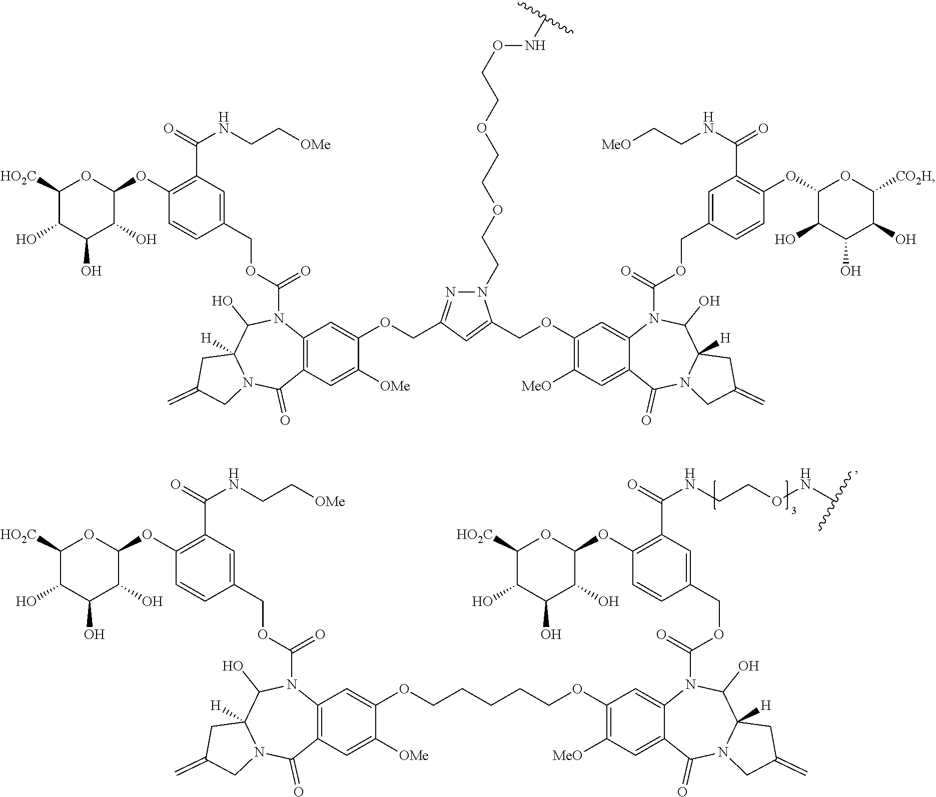 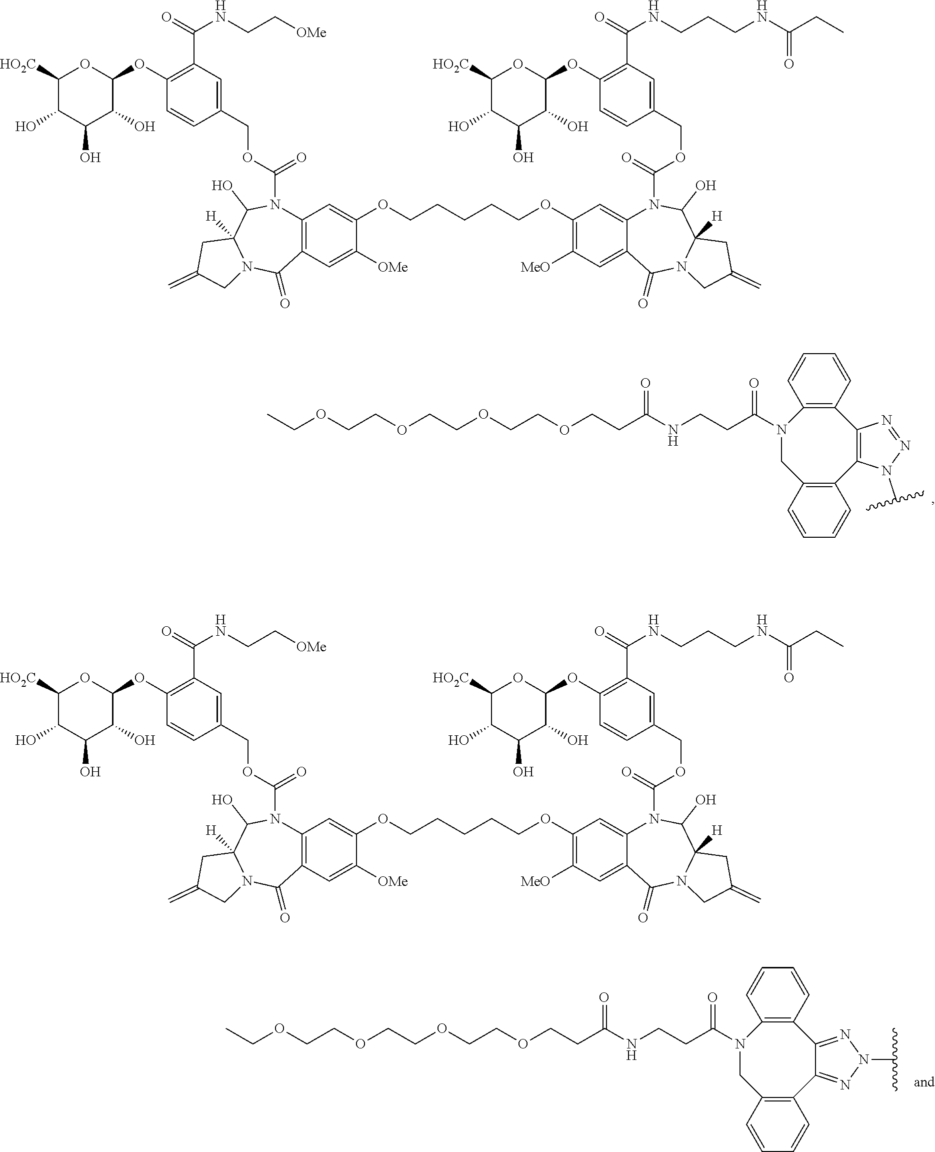 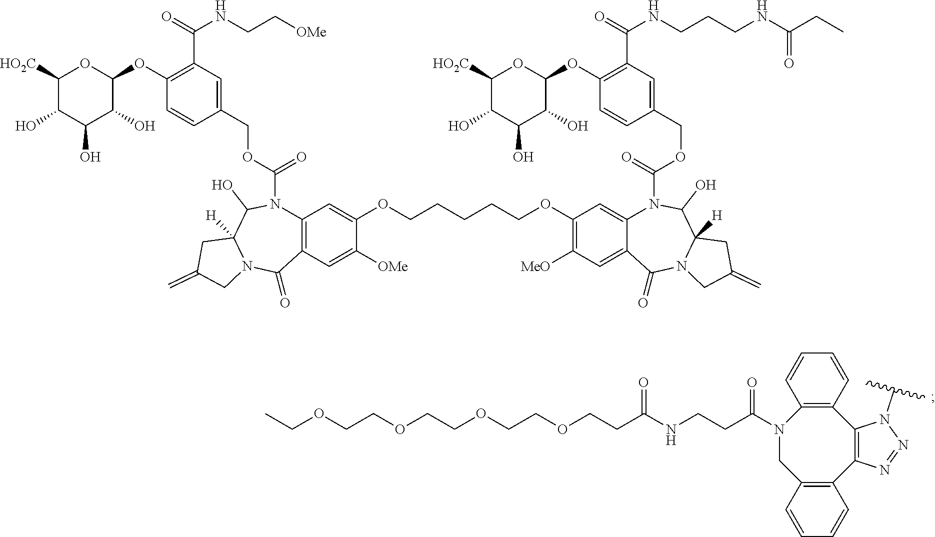
wherein MMAE is monomethyl auristatin E;
MMAF is monomethyl auristatin F; and
the wavy line represents connection point to the conjugate.

126. A pharmaceutical composition comprising the conjugate of any one of claims 1-125 and a pharmaceutically acceptable excipient.

127. A pharmaceutical composition for the prevention or treatment of hyperproliferation, cancer or an angiogenic disease, the pharmaceutical composition comprising the conjugate of any one of claims 1-125.

128. The pharmaceutical composition of claim 127, further comprising a pharmaceutically effective amount of a chemotherapeutic agent.

129. The pharmaceutical composition of claim 127 or 128, wherein the cancer is selected from lung cancer, small cell lung cancer, gastrointestinal cancer, colon cancer, bowel cancer, breast cancer, ovarian cancer, prostate cancer, testicular cancer, liver cancer, kidney cancer, bladder cancer, pancreatic cancer, brain cancer, sarcoma, osteosarcoma, Kaposi's sarcoma, and melanoma.

130. A pharmaceutical preparation comprising the conjugate of any one of claims 1-125 and a pharmaceutically acceptable carrier, the pharmaceutical preparation being selected from injections, tablets, pills, powders, granules, capsules, troches, suspensions, liquids for internal use, emulsions, syrups, emulsions, freeze-dried preparations, and suppositories.

131. A method of treating cancer or an angiogenic disease in a subject in need thereof comprising administering a conjugate of any one of claims 1-125 or a pharmaceutically acceptable salt thereof to the subject.

132. The method of claim 131, wherein the method treats cancer.

133. The method of claim 131 or 132, wherein the cancer is lung cancer, small cell lung cancer, gastrointestinal cancer, colon cancer, bowel cancer, breast cancer, ovarian cancer, prostate cancer, testicular cancer, liver cancer, kidney cancer, bladder cancer, pancreatic cancer, brain cancer, sarcoma, osteosarcoma, Kaposi's sarcoma, or melanoma.
